(19) United States
(12) Patent Application Publication
(10) Pub. No.: US 2013/0211188 A1
(43) Pub. Date: Aug. 15, 2013

(54) METHOD FOR INCREASING DISTENSIBILITY IN A GASTRIC BAND

(71) Applicants: MATTHEW G. FISHLER, Santa Cruz, CA (US); LILIP LAU, Los Altos, CA (US); MATTHEW J. PHILLIPS, Foster City, CA (US)

(72) Inventors: MATTHEW G. FISHLER, Santa Cruz, CA (US); LILIP LAU, Los Altos, CA (US); MATTHEW J. PHILLIPS, Foster City, CA (US)

(73) Assignee: CAVU MEDICAL, INC., Menlo Park, CA (US)

(21) Appl. No.: 13/397,322

(22) Filed: Feb. 15, 2012

(51) Int. Cl. A61F 2/04

(52) U.S. Cl. 600/37

(57) ABSTRACT

A gastric band assembly has one or more bladders incorporated therein so that the distensibility of the gastric band assembly is increased. The distensibility relates to the relative strength with which the gastric band assembly with a bladder resists the application of additional band contact pressure. Distensibility is quantified by measuring the change in band contact dimension (e.g., diameter or area) versus the change in band contact pressure.

### BACKGROUND

### Field of the Invention

The present invention relates to the field of treating obesity using an adjustable gastric band. As the patient loses weight, the gastric band is adjusted to accommodate for changes in weight.

Laparoscopic adjustable gastric banding (LAGB) was rapidly embraced as a procedure for treating morbid obesity after its introduction in Europe and in the United States. Compared to Roux-en-Y gastric bypass, the existing gold standard bariatric surgery procedure, it was attractive because it was safer, with one-tenth the peri-operative mortality, less morbid, easier and faster for surgeons to learn and perform, required a shorter hospital stay and resulted in a faster post-operative recovery. In addition, the device and the degree of restriction that it provided could be adjusted to suit the patient at different points in time. If necessary, the device could be removed surgically. The procedure involves no permanent alteration of the patient's anatomy. In addition, the patients are free of many of the side effects that accompany gastric bypass such as hair loss, anemia and the need to take supplemental vitamins. These attributes were attractive both to the health care providers and to the patients.

However, laparoscopic adjustable gastric banding has some drawbacks. Weight loss and co-morbidity resolution do not occur as rapidly as with gastric bypass surgery, with most reported results trailing in weight loss at one, two, three and possibly four years. In addition, there is considerably more variability from patient to patient in the amount of weight that they lose. More recent data has suggested that over time, the difference diminishes because gastric bypass results show an early peak in weight loss followed by subsequent decline. At five years there does not appear to be a statistical difference in weight loss between bypass and gastric banding (Surgery for Obesity and Related Diseases 1, pp. 310-316, 2005).

One current method for treating morbid obesity includes the application of a gastric band around a portion of the stomach to compress the stomach and create a narrowing or stoma that is less than the normal interior diameter of the stomach. The stoma restricts the amount of food intake by creating a pouch above the stoma. Even small amounts of food collecting in the pouch makes the patient feel full. The patient consequently stops eating, resulting in weight loss. It is important to maintain the right level of restriction imparted by the band in order for the patient to feel full and thereby to have continuous and uniform weight loss. Prior art gastric bands include a balloon-like section that is expandable and deflatable by injection or removal of fluid from the balloon through a remote injection site such as a port near the surface of the skin. The balloon expandable section is used to adjust the correct level of restriction imparted by the band both intraoperatively and postoperatively. Currently, patients must return to the doctor as many as four to ten times per year for several years in order to have fluid injected into or removed from the balloon in order to maintain the correct level of restriction imparted by the band.

It was first reported by Forsell and colleagues in 1993 (“Gastric banding for morbid obesity: initial experience with a new adjustable band”; Obes. Surg. 1993; 3:369-374) that individuals with adjustable gastric bands experienced plateaus in their weight loss during the time between scheduled adjustments. A typical weight loss curve is shown in FIG. 1A.

In 2008, Rauth, et al. (“Intra-band pressure measurements describe a pattern of weight loss for patients with adjustable gastric bands”; J. Am. Coll. Surg. 2008; 206; 5:926-932) reported that “patients commonly attribute this pattern of weight loss to a ‘loosening’ of their band, stating that the band provides progressively less restriction during meals and less satiety between them.” Rauth, et al. described a clinical study that uses a manometer to measure the intra-band pressure of the adjustable gastric bands in vivo during routine postoperative adjustments. The group recorded significant intra-band pressure drops between adjustments and proposed that such loss of band pressure, which could not be explained solely by band volume loss, not intra-band volume, led to plateaus in weight loss and results in patients' observations that the band becomes looser with time as shown in FIG. 1B.

Rauth, et al. suggested that the loss of band pressure was due to remodeling of the tissue that is occupied by the inner circumference of the band. They hypothesized that during the first 60 days after band insertion, there remains considerable perigastric fat and some residual tissue edema; the volume of the encircled stomach is greatest. As weight is lost and edema resolves, the volume of stomach contained within the band decreases, resulting in less contact pressure between the tissue and the band which in turn results in a decrease in intra-band pressure.

In order to be efficacious and safe, frequent follow-up visits to the physician, most of which involve band adjustments, are necessary. Some have described this as the Achilles heel of gastric banding. In fact, studies have shown a correlation between weight loss and the number of band adjustments or office visits that a patient undergoes (Shen). The band adjustments are usually performed in the setting of a physician's office. In these procedures saline is added or removed from the band in order to adjust it to the right tightness or restriction. Many factors are considered in making this adjustment. The goal is to try and tune the band to a “sweet spot” or “Green Zone.” In this zone the patients are able to adhere to proper eating patterns and lose one to two pounds per week. Burton et al. described the relationship of fluid volume in the gastric band and its effect on intra-luminal pressure to cause changes in the patients' clinical states (Burton, Paul R., et al., Effects of Gastric Band Adjustments on Intraluminal Pressure, OBES. SURG., 19:1508-1514, 2009). Burton, et al. showed that in successful patients, presumably those in the Green Zone, the basal intra-luminal pressure at the level of the LAGB was consistently at or near the range of 15-35 mmHg despite patients having different bands. Furthermore, the amount of intra-band volume required to achieve this Green Zone pressure range was variable and dependent on the individual patient but usually fell within a narrow range of about 1 mL for a given patient. This appears to be a physiological target for proper band adjustment and maintenance. That is, regardless of band type or fill volume it is important to achieve and maintain an intra-luminal pressure in or near the range of 15-35 mmHg. It is noted that during swallowing, the intra-luminal pressure can be much higher than the Green Zone pressure, but it is only temporary.

### Gastric Band Adjustment To Optimize Weight Loss

| YELLOW ZONE | GREEN ZONE | RED ZONE |
|---|---|---|
| Add Fluid | Fluid Level Optimum | Remove Fluid |
| Patient is hungry | Patient not hungry, good | Patient makes poor food |
| between meals, | weight loss, food portion | choices, experiences |
| eating large | control, patient satisfaction | regurgitation, discomfort |
| portions, and | | while eating, poor weight |
| not losing weight | | loss, night coughing |
| Not enough fluid | Right amount of fluid in the | Too much fluid in the |
| in the band | band | band |

Current gastric band adjustment protocols vary from physician to physician and also depend on the feedback provided by the patient. Most physicians currently leave the band empty for the first six weeks or so after the surgery in order for the band to heal in place. The healing involves a foreign body response in which inflammation and fibrosis lead to encapsulation of the band. Typically, this process subsides over time in the absence of further stimulation. After this initial settling in period adjustments to the band begin. Adjustments typically can be categorized into two phases: the initial careful incremental adjustment into the Green Zone followed by the subsequent maintenance of the Green Zone by tuning the band to either tighten or loosen it to achieve the desired restriction. Conventional adjustment practice involves adding or removing prescribed increments of saline (e.g., 0.5 cc) to the band and then double checking the level of restriction by having the patient sit up and drink water or barium under fluoroscopic imaging. In the initial phase increments of saline are added up to or starting from a target volume (e.g., 4 cc). As can be expected, there is considerable patient to patient variability as to the intra-band volume and number of adjustments that initially bring them into the proper adjustment of the Green Zone. Typically, within the first few weeks of receiving an LAGB, two to five adjustments are needed to attain the Green Zone initially.

It is important to note that the values of intra-band pressure associated with the Green Zone as used herein, are representative numbers that may vary in actual practice on patients. What is important is that once a doctor finds a band setting that is optimal for weight reduction for that patient, then that is the Green Zone. Thus, the intra-band pressure range associated with the Green Zone includes a target pressure directly or indirectly set by the doctor during a band adjustment, and the longer a patient stays in the zone while losing weight, the fewer the number of adjustments on the band are required to keep the patient in the zone for optimal weight loss. Some doctors make band adjustments by adding fluid volume to the balloon portion of the band, but they do not actually measure the intra-band pressure. While these doctors may not measure the intra-band pressure, they may get feedback from the patient (swallowing, tightness, etc.) to set the intra-band pressure at a pressure level for optimal weight loss. This unmeasured pressure level also is considered in the Green Zone and the goal is to keep the patient at or near this set intra-band pressure level for as long as possible before requiring another adjustment. With this latter method, however, the doctor does not know the actual intra-band pressure setting.

Once the patients attain the Green Zone, subsequent adjustments are performed to keep them there. In the first year after band implantation there may be five or more additional adjustments to attain the Green Zone. Most often this involves adding saline or tightening the band on a monthly or so basis. This is performed if the patient falls out of the Green Zone. More commonly this is in response to inadequate rate of weight loss which often coincides with patients reporting that their bands have loosened or are loose (patient is in the Yellow Zone). The exact mechanism behind the loosening is not clear, but several factors have been suggested. Some leakage of saline may occur out of the band over time. Air is often trapped in the band initially which may dissolve or dissipate over time. Epi-gastric fat is often encircled by the band and with time this may go away. The stoma itself and the fibrous cap around the band may remodel over time. What is clear though is that the addition of sometimes small amounts of saline into the band will bring back the feeling of restriction to the patients.

Occasionally, gastric bands need to be loosened as well. If the band is too tight or tightened too quickly the patient may feel excessive restriction. The patient may have a difficult time eating with frequent episodes of vomiting (patient is in the Red Zone). Also, certain foods may get stuck. Ironically, this may lead to weight gain as patient learns to cheat the restriction provided by the band by drinking milkshakes and other liquid foods. Another more serious drawback of excessive tightening is that the band may erode through the stomach wall if it is left in that state. Swelling or edema can cause the band to become too tight. Patients report that bands may be tighter feeling in the morning and looser later in the day. Female patients often report feeling increased tightness around the time of their menstrual cycles. Usually, removing fluid from the band can relieve this tightness.

Band adjustments are still performed beyond the first year but less frequently. Patients may come in on a quarterly basis, especially during the second and third year.

Despite the recognition of the criticality of band adjustments, patient compliance remains an issue. Some patients may not come in for adjustments when required. Many patients live considerable distances from the surgeon who implanted their band. The need for frequent adjustments can be very demanding on these patients in terms of the time away from work and cost of travel. In the extreme case, many patients opt to have their bands implanted out of the country because of cheaper costs. After their procedure they cannot afford to travel out of the country for frequent band adjustments. Some patients move and subsequently have difficulty finding a surgeon to perform their adjustments. Even within the U.S. some surgeons will not adjust the bands of patients that were not implanted by them for fear of potential liability.

Further, there is the direct cost of adjustments. Typically, even when the surgery is reimbursed by insurance, the adjustments are not, or even when they are, they are inadequately reimbursed. The patient may not be able to afford the out-of-pocket fees for adjustments which often can be several hundred dollars per adjustment. Finally, there are complex psychological motivational obstacles that prevent them coming in for the necessary adjustments. For example, some patients have a fear of the syringe needle that is used to inject saline into the band.

The inconvenience of adjustments is not limited to the patients. Surgeons generally do not like the need for frequent adjustments. Historically, they are not accustomed to the intensive long term care of their patients. Many do not have the existing infrastructure within their practices to manage the post-procedural aftercare of the patients. This consists of having the staff to perform adjustments, providing counseling, psychologists, nutritionists, nurses, etc. In addition, as surgeons implant more and more bands, the pool of patients that will need adjustments grows. Consequently they may end up spending less time operating and a considerable amount of time performing adjustments.

Without adjustments patients experience interrupted or cessation of weight loss and even weight regain. If the bands are too loose the patients' eating habits may regress. Even if they are aware of this it often can take time for them to schedule and receive a proper adjustment. If the bands are too tight and not adjusted they not only are uncomfortable, but patients may adopt bad eating habits, such as drinking milkshakes. In the extreme case patients can experience erosion of their stomach or esophagus by their bands which would necessitate band removal.

Even if the patients are compliant and can overcome the barriers to attending follow-up visits adjustments can be problematic. Locating the subcutaneous fill port can be difficult. Sometimes the port will move or flip over. In these cases fluoroscopy or even surgical revision are needed. Repeated needle punctures can lead to infection. Actual adjustment protocols can differ from surgeon to surgeon. Different bands have different pressure-volume characteristics which can lead to even greater inconsistency. The adjustment protocols were derived from trial and error and not any physiological basis. Even after a patient is properly adjusted changes may occur very shortly afterward, within days to weeks, that create a need for another adjustment.

It is clear that the less the need for adjustments the better the gastric banding therapy will be. Weight loss results will be more uniform from patient to patient and less dependent on follow up. The amount of weight lost and the rate at which it is lost will also be better because of less interrupted weight loss. Co-morbidity resolution will also improve accordingly. Less need for band adjustments would also result in cost and time savings to both the patients and healthcare providers. Reducing the variability in outcomes, increasing the rate and amount of weight loss and reducing the need for follow-up visit adjustments combined with the inherent present advantages of gastric banding would create a bariatric surgery potentially that would offer the best of gastric bypass and banding. Many more patients may opt for this procedure than previously would have chosen bypass or banding.

Current band adjustments are highly variable if measured in terms of volume, which is the current adjustment metric. Rauth, et al.'s group reported substantial variability in intra-band volume that can produce similar intra-band pressure as shown in FIG. 1C. Patient #39's intra-band pressure reached 730 mmHg at the intra-band volume of 2 mL while patient #43's intra-band pressure reached similar level (758 mmHg) at the intra-band volume of 4 mL, a difference of 2 mL which is 50% of the entire intra-band volume capacity (see FIG. 1C).

Also, other published papers suggest that a narrow range of intra-band pressure based on a more physiological approach might achieve good weight loss and prevent esophageal problems in the long term. Lechner and colleagues (“In vivo band manometry: a new access to band adjustment”; Obes. Surg.; 2005; 15:1432-1436) reportedly adjusted a cohort of twenty-five patients to a basic pressure of 20 mmHg at the first band filling. None of the patients returned to the clinic due to obstruction. In a continuation of this work, Fried reported that when patients that had previously lost less than 40% EWL with banding, they were adjusted to 20-30 mmHg intra-band pressure using manometry, resulting in significant weight loss at 12 weeks. Both Lechner, et al. and Fried, et al. suggested that the gastric band adjustment based on pressure might be more physiologic, accurate and reliable. Furthermore, Gregersen in his book titled “Biomechanics of the Gastrointestinal Tract” stated that the normal resting pressure “in the lower esophageal sphincter generally lies between 10 and 40 mmHg above atmospheric pressure.” Thus, it would seem reasonable to have band-tissue contact pressure near this range.

One drawback common among the prior devices that use some type of device to fill and replenish fluid in the balloon portion of the band is that their pressure-volume compliance curves are relatively steep. In other words, for each incremental fill volume (i.e., 0.5 mL), there is a correspondingly large increase in intra-band pressure. Published prior art pressure volume curves are disclosed in Ceelen, Wim, M. D., et al., Surgical Treatment of Severe Obesity With a Low-Pressure Adjustable Gastric Band. Experimental Data and Clinical Results in 625 Patients, Annals of Surgery, January 2003, pp. 10-16; Fried, Martin, M. D., The current science of gastric banding: an overview of pressure—volume theory in band adjustments, Surgery for Obesity and Related Diseases, 2008, pp. S14-S21; Rauth, Thomas P., M. D., et al., Intraband Pressure Measurements Describe a Pattern of Weight Loss for Patients with Adjustable Gastric Bands, Journal of American College of Surgeons, 2008, pp. 926-932; Lechner, Wolfgang, M. D., et al., In Vivo Band Manometry: a New Access to Band Adjustment, Obesity Surgery, 2005, pp. 1432-1436; Forsell, Peter, et al., A Gastric Band with Adjustable Inner Diameter for Obesity Surgery: Preliminary Studies, Obesity Surgery, 1993, pp. 303-306 which are incorporated herein by reference thereto.

Band adjustments are made by a physician by adding or removing fluid from the band. Typical adjustment volumes for different gastric bands are listed below (presented by Dr. Christine Ren at the Band Summit 2009). As the data indicates, it typically takes many adjustments to bring the patient into the Green Zone. Also, the data shows that larger volumes of fluid are added initially. As the patient approaches the Green Zone, the band becomes very sensitive to small volume adjustments. This means that a small amount of fluid added to the band can bring the patient in or out of the Green Zone. The requirement for multiple adjustments has become a major burden to the patients as well as to the physicians.

| Band Type | Fluid Volume (mL) |
|---|---|
| 9.75/10 | 0, 1, 1, 0.5, 0.2, 0.1 |
| VG | 3, 3, 2, 1, 0.5, 0.2, 0.1 |
| APS | 3, 2, 1, 0.5, 0.2, 0.1 |
| APL | 4, 2, 1, 1, 1, 0.5, 0.2, 0.1 |
| REALIZE | 0, 3, 2, 1, 1, 0.5, 0.2, 0.1 |
| REALIZE-C | 0, 4, 2, 1, 1, 0.5, 0.5, 0.2 |

*Range +/−1 cc depending on amount peri-gastric fat within band

This phenomenon is also mentioned by Burton, et al. in the paper titled “Effects of Gastric Band Adjustments on Intra-luminal Pressure.” In the study, Burton, et al. suggested that there might be direct correlations between the intra-luminal pressure underneath the band and the different clinical states. In particular, intra-luminal pressure between 15-35 mmHg represents the Green Zone clinical state for most patients. Furthermore, Burton, et al. also observed that the Green Zone is represented by a narrow range of fluid volume, around 1 mL for most patients. A graph of intra-luminal pressure vs. intra-band volume of three different banding patients illustrated his finding and is shown in FIG. 1H. It took about 1 mL of fluid to increase the intra-luminal pressure from 15 mmHg to 35 mmHg (the range of the Green Zone) for all three patients. This finding offers a plausible explanation to the clinical observation that the band becomes very sensitive to small volume adjustments when the patient approaches the Green Zone.

Regardless of band type and investigator there appears to be a common finding in the prior art of an intra-luminal or intra-band pressure threshold for safe band adjustment. This threshold appears to be somewhere in the range of 20-40 mmHg intra-luminally. Adjustment of bands above this intra-luminal pressure threshold results in too tight of a band. Over tightened bands correspond to a clinical state referred to as the “Red Zone.” In the Red Zone patients have difficulty swallowing food, especially solid food. Food gets stuck easily within the stoma formed by the band. This is known as bolus obstruction. This results in dysphagia, reflux, regurgitation, pouch dilatation and can result in maladaptive eating all of which lead to unsatisfactory weight loss.

Support for this threshold comes from several reported studies. Udomsawaengsup et al. (SOARD 3: (2007); 296) reported on a series of intra-band pressure measurements in which the patients who required readjustment due to obstructive symptoms had intra-band pressures greater than 55 cm H₂O (40 mmHg). Fried et al. (SOARD 4 (2008) S14-S21) found that adjusting patients to a “mean band pressure sufficient to exert a significant yet not disruptive restriction” of 20 mmHg resulted in no patients requiring readjustment due to obstruction. Lechner et al. (Obes Surg (2005) 15, 1432-1436) identified an intra-band pressure threshold, mean pressure of 25.5 mmHg, (range 15-55 mmHg), that appeared to be the level at which obstruction occurred. The optimum range to set a band appeared to be just below this threshold. Patients were adjusted to a basic pressure of 20 mmHg. The corresponding ex vivo pressure at equivalent volume was 4 mmHg which suggests a 16 mmHg contact or intra-luminal pressure was generated. Burton et al. (Obes Surg (2009) 19:1508-1514) found that in patients who were in the Green Zone the intra-luminal pressure fell within a relatively narrow range of pressures from 15-35 mmHg. Above this range patients were likely to fall into the Red Zone, meaning that the bands were overfilled and prone to obstruction.

Avoidance of over tightening a band is important but some level of tightness is necessary in order for the band to be effective. The “Yellow Zone” is commonly used to refer to too loose of a band. In this state the patients are able to eat freely and do not have sufficient satiety induction as a result of eating. Consequently the patients remain hungry and have unsatisfactory weight loss.

In order for a band to be effective it must be sufficiently tight to create a state referred to as the Green Zone. Here the patients feel lasting satiety as a result of eating. It is believed that the band induces mechano-sensory stimulation to the gastric tissue and nerves in the vicinity of the band and that these are responsible for satiety induction.

Gao et al. (Obes. Surg. (2008) 18:243-250, performed a study in silico in which they simulated the effects of varying stoma size on stomach pouch wall stress during swallows. They found that the maximum stress in the stomach pouch increases as stoma size is reduced. Usually, the more filled a band the smaller the corresponding stoma size. Furthermore, the higher the level of stress or stretch experienced by the stomach pouch the greater the level of mechano-sensory stimulus can be expected. Thus the tighter the band, the more satiety induction can be expected for a given patient and among patients. The greater the intra-band pressure and volume the tighter a band will be.

Currently, the level of band tightness is limited by the need to avoid bolus obstruction by the band during swallowing of food. If the intra-band pressure threshold at which bolus obstruction occurs were higher, bands could be filled to a tighter level at higher pressures. This may induce a greater level of satiety and do so in a greater proportion of patients. This would also make adjusting bands to the desired level easier by increasing the effective pressure.

Several studies have characterized the pressure behavior of current LAGB during swallowing. (Burton, Lechner, Fried). An esophageal pressure wave normally propels the food down the esophagus to the gastric pouch above the band. The successful transit of food through the band during swallowing depends on the resistance created by the band, consistency of the food and the motility of the esophagus. The narrowing of the stomach lumen, or stoma, formed by the band creates a resistance to the passage of this bolus. The level of resistance is a function of the size and the distensibility of the stoma as well as the intra-luminal or inward contact pressure generated by the band. The intra-luminal pressure is at least partially a function of the intra-band pressure and volume. Depending on the consistency of the food bolus, different amounts of bolus pressure may be required to cause food to pass through the stoma. Liquids may pass through easily. Solid foods typically require greater or more esophageal pressure magnitude to push the bolus through the resistance imparted by the band.

The higher the intra-luminal pressure within the stoma the greater the resistance to the passage of a bolus. When this bolus pressure exceeds the intra-luminal pressure at the level of the band, food passes through. Often food will not pass through because of insufficient bolus pressure. Also, the bolus may partially pass through. In response to residual bolus the esophagus will generate secondary waves in an attempt to push food through. This may result in reflux or regurgitation as the path of least resistance to the flow of the food bolus is in the reverse, retrograde direction.

When food gets stuck within the band there can be a resulting rise in basal or resting (not referring to active contraction of the esophagus) intra-band pressure. Repeated secondary pressure waves are automatically generated in the esophagus in an attempt to clear the obstruction. The ability to clear an obstruction is primarily affected by four things: the bolus pressure that the esophagus can generate to push the obstructed food, the degree of resistance generated by the band, the compressibility of the bolus itself (for example liquid or semi-liquid can change configuration and ease its way through), and the ability of the stoma (band) to enlarge to allow the bolus to pass through. For a given food consistency and esophageal pressure generated, or motility, the resistance to food passage is governed by a number of band related variables: the diameter of the stoma, the basal intra-band and contact pressure and the compliance of the band. The larger the stoma diameter, the lower the intra-band pressure and the more compliant the band, the easier it is for food to pass or an obstruction to clear.

As food gets lodged within the stoma, multiple secondary waves are generated to push the food though. A larger stoma size means that food is less likely to get stuck and even if it does, secondary waves have a better chance of advancing the food through the stoma. The higher the intra-band pressure the higher the intra-luminal pressure that the food and esophagus must overcome in order to pass through the stoma, both initially and after the bolus gets lodged. The more compliant the band the more it can change shape and enlarge in response to increased pressure from within the stoma. It would take less esophageal energy, a function of pressure and time or number of contractions, to cause a given stoma size change with a more compliant band. Hence a more compliant band will require less pressure and fewer secondary contractions in order for food to pass through and especially for food to become dislodged.

Existing bands have insufficient fluid capacitance so that the diameter enclosed by the band cannot increase significantly to allow the bolus to clear. This may be true even if the intra-luminal/stoma pressures are low to begin with. They have limited capacitance because the fluid in the band is incompressible and the silicone rubber only has limited ability to stretch. Furthermore there is nowhere for the intra-band fluid to be displaced. It may take exceedingly high pressures, which cannot be generated by the esophagus, to enlarge the stoma significantly. Repeated or frequent high pressures may be the cause of esophageal dilation and or exhaustion, one of the purported shortcomings of the LAGB procedure. The smaller the starting stoma and higher the starting intra-band pressure the more bolus pressure from the esophagus will be required to push food through the stoma. This is unless the capacitance of the band is increased significantly.

Stoma distensibility is an area related to compliance/capacitance, but not addressed in the prior art. In practice, an implanted LAGB is titrated with a quantity of fill volume (saline) with the intent of maximizing positive therapeutic effects (e.g., weight reduction, satiety, etc) while minimizing negative adverse effects (e.g., vomiting, obstruction, etc). Bands that are properly adjusted within this therapeutic “sweet spot” are considered to be in the Green Zone. Bands that are under-filled (insufficient therapy) are said to be in the Yellow Zone while Bands that are over-filled (excessive adverse effects) are said to be in the Red Zone. Burton, et al. (Burton, P. R. et al., 2009. “Effects of gastric band adjustments on intraluminal pressure,” Obesity Surgery, 19(11), p. 1508-14) showed that in successful patients (presumably those in the Green Zone), the basal intra-luminal pressure at the level of the LAGB was consistently at or near the range of 15-35 mmHg despite patients having different bands. When basal intra-luminal pressure was <15 mmHg, patients were able to eat freely, and consequently weight loss was unsatisfactory. In contrast, when basal intra-luminal pressure was >35 mmHg, patients demonstrated obstructive symptoms including dysphagia, reflux, regurgitation, etc. Thus, according to this study, this intra-luminal pressure range appears to be a physiological target for proper band adjustment and maintenance. That is, regardless of band type or fill volume, it is important to achieve and maintain a basal intra-luminal pressure in or near the range of 15-35 mmHg.

In their discussion, Burton, et al. posit that the likely reason that few LAGB patients exceed a basal intraluminal pressure of 35 mmHg is that it is simply beyond the capacity of the esophagus to transit solid food across the LAGB at those elevated intra-luminal pressures. Implied in this statement is the notion that, when the LAGB is “over-filled” such that it induces these elevated Red Zone intra-luminal pressures, the distensibility of the LAGB (or, perhaps more comprehensively, the stoma at the level of the LAGB) is insufficient to allow the stoma to open enough—even at the maximal intra-luminal swallow pressures generated by the esophagus—to enable the food bolus to pass through it.

Interestingly, a recent publication distributed by Ethicon Endo-Surgery, Inc., entitled “Pressure Guided Gastric Band Adjustments” (publication number DSL 11-0534.GH© 2011) enumerates multiple factors that impact the transit of luminal contents through the LAGB. Notably absent from this article is stoma distensibility. Thus, it appears that stoma distensibility has not yet been explicitly recognized in the prior art as a variable with respect to LAGB performance vis-à-vis successful vs. unsuccessful swallow performance.

What is needed is a device and method for use with a gastric band to set the intra-luminal pressure higher than that disclosed in the prior art devices and to maintain the higher pressure as long as possible between adjustments. What has been required in the art is a device that automatically adjusts the fluid level in the gastric band to maintain it and the entire system at or near the intra-band and/or contact pressure at which the band was last adjusted to. The present invention provides a device for passively equalizing pressure in a closed fluid system that automatically and continuously tries to equalize the pressure in the system in order to maintain the proper restriction to keep the patient in a prescribed intra-luminal pressure range that is higher than that disclosed in the prior art. The device of the present invention provides increased capacitance and thus distensibility (for a given band compliance) such that even when set at even higher intra-band pressures, the stoma created by the band can increase with response to food being stuck and thus allow the food obstruction to clear.

Further, the system and methods described herein provide a means to increase the distensibility of a LAGB. With such enhanced distensibility, it may be possible to expand or enhance the LAGB therapeutic Green Zone by enabling further maximization of positive therapeutic effects and/or further minimization of negative adverse effects.

### SUMMARY OF THE INVENTION

One embodiment of the invention relates generally to the treatment of obesity using a gastric band or lap band that encircles a portion of the stomach thereby producing a stoma which limits the amount and/or rate of food intake by the patient. The gastric band has an adjustable fluid balloon which can be expanded or deflated in order to provide the right level of restriction, compression, pressure or narrowing to the stomach of the patient. Importantly, this embodiment provides for a bladder in fluid communication with the balloon that increases the effective distensibility of the LAGB contact area/diameter, and hence also the effective distensibility of the stoma encircled by the gastric band, as compared to the LAGB balloon alone. The gastric band or system distensibility refers to the rate of band contact dimensional change per unit change in applied band/system contact pressure (i.e., ΔSD/ΔSP) typically under the assumption of a constant total band/system fill volume. The band contact dimension can be any of a band contact diameter, circumscribed band contact area, or any other relevant dimensional description of the stomach region encircled by, and hence in contact with, the balloon portion of the band. Distensibility functionally relates to the relative strength with which the gastric band assembly resists the application of additional contact pressure as generated from/by the stomach at the level of the band. This additional contact pressure imparts a net-outwardly radial force to the gastric band balloon that causes the physical configuration of the balloon to deform away from its lowest viable energy state for that given fill volume. This change in physical configuration is generally measured as the band contact dimension (e.g., diameter, circumscribed area), and therefore distensibility (D) can be quantified as D=ΔSD/ΔSP. In one embodiment, one or more passive compliant bladders that are separate from, yet in continuous direct fluid communication with, the LAGB balloon increases the effective distensibility of the band contact dimension. The definition of “stoma area” is the intra-luminal opening inside that portion of the stomach tissue encircled by the balloon portion of the gastric band. The definition of “band contact area” is the area of stomach tissue encircled by the balloon portion of the gastric band and includes the stoma area. Under resting (basal), non-contracting conditions, changes in the basal intra-luminal pressure generally result in corresponding changes in basal contact pressure and basal intra-band pressure (i.e., all pressures go up or go down). “Basal” pressure is defined as the pressure when resting, i.e., not swallowing or otherwise causing the pressure to fluctuate.

In one embodiment, one or more bladders are incorporated in an existing LAGB system to increase the distensibility of the band contact dimension. In this embodiment, for the same increase in applied band contact pressure the addition of the bladder of the present invention to a conventional LAGB increases the band's contact dimension distensibility as compared to a LAGB only assembly. Importantly, this increased distensibility over a conventional LAGB enables the band contact dimension of a system with the LAGB plus a bladder to open to a substantially larger dimension for any given increase in applied band contact pressure (e.g., as generated by the esophagus during swallowing). The LAGB with a bladder of the present invention is able to successfully accommodate larger food boluses within a person's swallowing capability without obstructing or inducing other obstructive symptoms (e.g., blockage, vomiting, dilatation, etc.). In this embodiment, the one or more bladders provide increased distensibility to the band contact dimension enabling the band contact dimension to open by a given amount with substantially less required increase in band contact pressure (i.e., swallowing pressure transient). It is postulated that very high swallowing pressures, even if the swallow is ultimately successful, might induce adverse effects such as pouch dilatation. This embodiment enables successful swallowing while reducing the possibility of pouch dilatation or other adverse effects because the increased distensibility enables the band contact dimension of the LAGB plus bladders configuration to open by a given amount with substantially less required increase in band contact pressure. As compared to a LAGB only configuration, the LAGB plus bladder configuration requires as little as ⅓ the amount of increase in applied band contact pressure to open a band contact dimension by the same amount as the LAGB only configuration.

In another embodiment, the increased distensibility enables the band contact dimension of the LAGB plus a bladder configuration to be set to a tighter basal dimension than that of an LAGB only configuration and still be opened to the same final band contact dimension for any given increase in applied band contact pressure. In this embodiment, comparing the LAGB only configuration to the LAGB plus bladder configuration, the latter can be set to a higher basal intra-band pressure yet the latter configuration is able to open to the same peak band contact dimension as achieved by the LAGB only configuration. With the bladder, a gastric band assembly would be able to accommodate a food bolus of a given maximal dimension from a tighter basal condition compared to that possible with an LAGB only system. It has been hypothesized that satiety signaling is enhanced by a tighter band setting, thus the gastric band assembly with a bladder configuration being set to a tighter basal dimension further improves the positive therapeutic effects while minimizing negative adverse effects.

In one embodiment, one or more bladders are provided and are in constant fluid communication with the expandable balloon-portion of the gastric band. The fluid volume in the bladders and the balloon automatically and continuously adjusts back and forth so that there is no lasting pressure differential between the expandable balloon and the bladders. In this embodiment, the one or more bladders provide a basal intra-band pressure sufficiently high to reduce the likelihood of gastroesophageal reflux disease (GERD). The high basal intra-band pressure, and hence high basal intra-luminal pressure, will prevent the backflow of stomach contents (e.g., gases, fluids, solids, acids, etc.) past the stoma area and into the esophagus, thereby effectively treating GERD.

### BRIEF DESCRIPTION OF THE DRAWINGS

FIG. 1 is a schematic of a prior art gastric band system depicting a balloon portion of the gastric band and fill port.

FIG. 1A depicts a typical prior art weight loss curve.

FIG. 1B depicts a typical prior art weight loss curve.

FIG. 1C depicts a graph depicting the variability in intra-band volume as it relates to intra-band pressure.

FIG. 1D depicts a graph of experimental data showing intra-band pressure dropping when a mandrel diameter encircling the band decreases.

FIG. 1E depicts a graph of intra-band pressure and volume curves resulting from experimental data.

FIG. 1F depicts a graph resulting from experimental data in which a bladder was incorporated between a gastric band a fluid infusion port.

FIG. 1G depicts a graph resulting from experimental data in which a bladder was able to change the intra-band pressure/volume characteristics of a gastric band.

FIG. 1H is a prior art graph depicting intra-luminal pressure vs. fill volume for an APS® gastric band.

FIG. 2 is a schematic view of a bladder assembly having elastomeric bands to add elasticity to the system.

FIG. 3 is a longitudinal sectional view of the bladder assembly of FIG. 2.

FIG. 3A depicts a graph of experimental data resulting from experiments on the bladder disclosed in FIGS. 2 and 3.

FIG. 4 depicts a schematic view of a bladder assembly encased in a housing.

FIG. 5A depicts a longitudinal cross-sectional view of one embodiment of the bladder assembly of FIG. 4.

FIG. 5B depicts a longitudinal cross-sectional view of an alternative embodiment of the bladder assembly of FIG. 4.

FIG. 5C depicts a graph of experimental data relating to the embodiment of the bladder shown in FIGS. 4, 5A and 5B.

FIG. 6 depicts a longitudinal cross-sectional view of a bladder assembly having multiple bladders encased in a housing.

FIG. 7 depicts a longitudinal schematic view of a bladder assembly having multiple bladders encased in a housing.

FIG. 8 depicts a longitudinal schematic view of multiple bladder assemblies aligned serially.

FIG. 8A depicts a graph of experimental data relating to the embodiment of the bladder shown in FIG. 8.

FIG. 9 depicts a schematic view of a bladder assembly housed in a fill port assembly.

FIG. 10 depicts a top cavity of the injection portion bladder assembly of FIG. 9.

FIG. 11 depicts a schematic view of a bottom cavity of the injection port bladder assembly of FIG. 9 with the bladder substantially unfilled.

FIG. 12 depicts an enlarged view of the bottom cavity of the injection port bladder assembly of FIG. 9 without a bladder.

FIG. 13 depicts an exploded schematic view depicting the top cavity and the bottom cavity of the injection portion bladder assembly of FIG. 9 with the bladder being substantially filled.

FIG. 14 depicts a schematic view of a bellows-type bladder assembly encased within a housing.

FIG. 15 depicts a longitudinal schematic view of a multi-compliant bladder assembly housed within a solid housing.

FIG. 16 depicts a multi-level pressure compliance curve associated with the multi-compliant bladder assembly of FIG. 15.

FIG. 17A depicts a schematic view of a gastric band assembly with a bladder assembly in form of tubing.

FIG. 17B depicts a cross-sectional view taken along lines 17B-17B showing a coaxial bladder and tubing assembly.

FIG. 17 C depicts a cross-sectional view taken along lines 17C-17C showing a bladder and tubing assembly having an elastic septum.

FIG. 18 depicts linearly increasing and decreasing compliance curves.

FIG. 19 depicts a flat or substantially constant pressure compliance curve.

FIG. 20 depicts a multi-staged substantially constant pressure curves.

FIG. 21 depicts multi-staged linearly increasing compliance curves.

FIG. 22A depicts a logarithmic increasing pressure compliance curve.

FIG. 22B depicts an exponentially increasing compliance curve.

FIGS. 23 and 24 depict a schematic view of a gastric band assembly with a bladder system and a sensor to monitor pressure or other parameters.

FIG. 25 depicts a schematic view of a bladder system incorporated into a venous access catheter assembly.

FIG. 26 depicts a schematic view of a gastric band assembly having an elastic balloon.

FIG. 27A depicts a plan view of a bladder having a longitudinal fold.

FIGS. 27B-27C depicts a cross-sectional view of the longitudinal fold of FIG. 27A; FIG. 27B shows the folded configuration and FIG. 27C shows the unfolded configuration.

FIGS. 28-30 depict multiple bladders connected serially by flexible tubing.

FIG. 30A depicts a schematic view of a gastric band assembly in which multiple bladders are connected at a distal end to the gastric band and at a proximal end to a refill port.

FIG. 31 depicts a schematic view of one bladder that is expanded.

FIG. 32 depicts a transverse cross-sectional view of the expanded bladder of FIG. 31.

FIG. 33 depicts a schematic view of a bladder in which the flexible tubing extends through the bladder.

FIG. 34 depicts a graph resulting from experimental data taken from a bladder with a mandrel.

FIG. 35 depicts a perspective view of a bladder having four wings (cross-shaped configuration).

FIG. 36 depicts an end view of a bladder having four wings and a flexible tubing extending into the bladder.

FIG. 37 depicts a side view of a deflated bladder having a winged configuration.

FIG. 38 depicts a side view of the bladder of FIG. 37 in which the bladder has been expanded with a fluid.

FIG. 39 depicts a transverse cross-sectional view taken along lines 39-39 of FIG. 38 depicting a bladder having four wings.

FIG. 40 depicts a transverse cross-sectional view of a bladder having four wings wherein the bladder is expanded from fluid and has tubing extending therethrough.

FIG. 41 depicts a transverse cross-sectional view of a bladder assembly having pre-stressed L-shaped portions attached by a silicone adhesive cap.

FIG. 42 depicts a pressure-volume curve generated by a bladder having a pre-stressed configuration.

FIG. 43 depicts a plan view of multiple bladders connected in series by flexible tubing in which the flexible tubing is shown in a bent configuration.

FIG. 44 depicts a pressure-volume curve relating to experiments with a gastric band and bladder assembly.

FIGS. 45A-45B depict a plan view of multiple bladders connected by flexible tubing in which the tubing is bent.

FIGS. 46A-46B depict a plan view of the minimum length of connecting tubing between bladders to permit the bladders to make a 180° turn.

FIG. 47 depicts a plan view of several bladders connected serially by bellows-shaped flexible tubing.

FIG. 48 depicts a plan view of the bladders in FIG. 45 in which the bellows-shaped flexible tubing is bent.

FIG. 49 depicts a plan view of a bladder having a radiopaque marker wire.

FIG. 50 depicts a cross-sectional view of the bladder in FIG. 50 in which the radiopaque wires are positioned in the valleys of the five-winged bladder.

FIG. 51 depicts a cross-sectional view of a bladder having radiopaque wires along the winged sections of the wing-shaped bladder.

FIG. 52 depicts a bladder under fluoroscopic imaging where no fluid is injected in the bladder so that the radiopaque wires are spaced close together.

FIG. 53 depicts the bladder of FIG. 52 under fluoroscopic imaging where 1 mL of fluid has been injected into the bladder thereby moving the radiopaque wires a distance apart.

FIG. 54 depicts the bladder of FIG. 52 under fluoroscopic imaging where 2 mL of fluid has been injected into the bladder thereby moving the radiopaque wires further apart.

FIG. 55 depicts the bladder of FIG. 52 under fluoroscopic imaging wherein 3 mL of fluid has been injected into the bladder thereby moving the radiopaque wires even further apart.

FIG. 56 is a partial cross-sectional view of a gastric band surrounding stomach tissue thereby forming a band stoma area and a stoma area.

FIG. 57 is a partial cross-sectional schematic view taken along lines 57-57 of a gastric band surrounding stomach tissue thereby forming a band stoma area and a stoma area.

FIG. 58 is a cross-sectional view taken along lines 58-58 depicting the band stoma area and stoma area encircled by the gastric band.

FIG. 59 is a schematic view of stomach tissue surrounded by the balloon portion of a gastric band thereby forming a band stoma area.

FIGS. 60A and 60B are schematics depicting an elastic sphere to illustrate compliance and distensibility characteristics.

FIG. 61 is a graph depicting data relating to distensibility of an LAGB stoma.

FIG. 62 is a graph depicting data relating to the distensibility of an LAGB-plus-bladder assembly.

FIG. 63 is a graph depicting data relating to the distensibility of an LAGB-plus-bladder configuration.

FIG. 64 is a graph depicting data relating to the distensibility of an LAGB-plus-bladder configuration.

FIGS. 65-67 are graphs of data relating to the distensibility of an LAGB only as compared to an LAGB-plus-bladder configuration.

FIGS. 68A-68B are graphs depicting net changes in stoma diameter for an Ethicon SAGB-VC plus bladder assembly.

FIGS. 69A-69B are graphs depicting net changes in stoma area for an Ethicon SAGB-VC plus bladder configuration.

FIGS. 70A-70B are graphs depicting net changes in stoma diameter for an Allergan Lap-Band AP Standard plus bladder configuration.

FIGS. 71A-71B are graphs depicting net changes in stoma area for an Allergan Lap-Band AP Standard plus bladder configuration.

FIG. 72 is a graph depicting a scatterplot of an LAGB change-in-pressure vs. change-in-volume pairings as measured in patients during follow-up visits.

FIG. 73 is a graph of data comparing an LAGB only configuration with an LAGB-plus-bladder configuration.

FIGS. 74A-74D are graphs summarizing the generalized comparison of contact pressure differences at point B vs. point C in FIG. 73.

FIGS. 75A-75D are graphs which summarize the generalized comparison of stoma diameter differences at point B vs. point D in FIG. 73.

FIGS. 76A-76-D are graphs summarizing the generalized comparison of contact pressure differences at point B vs. point C in FIG. 73.

FIGS. 77A-77D are graphs which summarize the generalized comparison of stoma diameter differences at point B vs. point D in FIG. 73.

FIG. 78 is a graph depicting swallowing simulation with the gastric band and bladders in the system.

FIG. 79 is an exploded perspective view depicting a flow restrictor of the present invention.

FIG. 80 is a perspective view depicting the flow restrictor of FIG. 79 as it is assembled.

FIG. 81 is a longitudinal cross-sectional view depicting the flow restrictor showing the ball seated in the ball seat thereby restricting flow through the main channel.

FIG. 82 is a longitudinal cross-sectional view depicting the flow restrictor where the ball is unseated and fluid can flow from the bladders through the main channel to the gastric band.

FIG. 83A is a longitudinal cross-sectional view of one embodiment of the flow restrictor depicting the ball seated in the ball seat thereby blocking fluid flow through the main channel from the gastric band to the bladders.

FIG. 83B is a transverse cross-sectional view taken along lines 83B-83B depicting the main flow channel and the bypass flow channel of the flow restrictor.

FIG. 84 is a longitudinal cross-sectional view depicting the flow restrictor of FIG. 84-84 in which the ball is unseated allowing fluid to flow from the bladders through the main channel to the gastric band.

FIG. 85 is a schematic view of a gastric band assembly which includes a restrictor positioned between the gastric band and the bladders.

FIG. 86 is a graph depicting the pressure variations due to patient swallowing with the band only, the band plus bladders, and the band plus bladders plus restrictor in the system.

FIG. 87 is a graph of prior art intra-band pressures vs. time during bolus wet swallows at different volume adjustments of an LAGB.

FIGS. 88A-88B are graphs of data points from bench-based in vitro experimental measurements of fluid flow through a flow restrictor.

FIGS. 89A-89C are graphs of data displaying three sets of temporal plots relating to internal pressures vs. time, fill volumes vs. time, and band stoma diameter vs. time.

FIGS. 90A-90D are graphs of data relating to a flow restrictor in combination with an LAGB and bladder configuration depicting progressive distensibility.

FIGS. 91A-91D are graphs of data relating to a flow restrictor in combination with an LAGB and bladder configuration depicting progressive distensibility.

FIGS. 92A-92C are graphs of data relating to the pressure-volume-diameter characteristics Allergan Lap-Band AP Standard.

FIGS. 93A-93C are graphs of data relating to the pressure-volume-diameter characteristics of an Ethicon SAGB VC.

FIG. 94 is a graph of data relating to a bladder having model no. C10-A.

FIG. 95 is a graph of data relating to a bladder having model no. C10-E.

FIG. 96A-96D are graphs of data relating to the distensibility of an Ethicon SAGB-VC.

FIGS. 97A-97C are graphs of data relating to the distensibility of an Ethicon SAGB-VC.

FIGS. 98A-98D are graphs of data relating to the distensibility of an Allergan APS gastric band.

FIGS. 99A-99C are graphs of data relating to the distensibility of an Allergan APS gastric band.

FIG. 100 is a graph illustrating the time-courses of various parameters affected by an LAGB having a bladder and symmetric flow restrictor.

FIGS. 101A-101D are graphs of data relating to the conductance of an LAGB having a bladder and symmetric flow restrictor.

FIG. 102 depicts a schematic view of an LAGB placed around simulated stomach tissue and having a bladder assembly and flow restrictor.

FIG. 103 is a graph of conductance profiles of asymmetric and symmetric flow restrictors.

FIG. 104A-104B are graphs of data for an LAGB-only configuration and an LAGB having a bladder and flow restrictor configuration.

FIG. 105 is a longitudinal cross-sectional view depicting a symmetric flow restrictor.

### DETAILED DESCRIPTION OF THE PREFERRED EMBODIMENTS

At present, typical prior art gastric banding systems include a gastric band having an expandable balloon section and constant diameter tubing extending from the balloon to a port. The port is implanted near the surface of the skin so that fluid can be injected into the port with a syringe in order to add fluid to the balloon section thereby adjusting the level of restriction. One such typical gastric banding system is disclosed in U.S. Pat. No. 6,511,490, which is incorporated by reference herein. As used herein, gastric band and lap band are interchangeable.

The disclosed embodiments generally include one or more bladders in constant fluid communication with the expandable balloon section of the gastric band to automatically and continuously minimize the drops or rises in pressure from the set point from the last adjustment and in doing so the proper level of restriction provided by the band in order to keep the patient at the pressure and/or stoma dimension set by the physician at the last adjustment. The bladders are a passive system that do not require motors, drive pumps, or valves, nor do they require a feedback sensor to measure pressure or the level of restriction and then make adjustments based on the sensed parameter. Forces acting on the band are balanced by forces generated by the bladder. These bladder forces are a function of compliance/design of the bladder and vary with the volume or fill state of the bladder. With the disclosed bladders, the pressure/volume relationship in the system is not adjustable, although pressures are adjustable by adding/removing volume as mentioned earlier, i.e., the bladders passively maintain an intra-band pressure range for a longer time period than with the gastric band alone. They do so by reducing intra-band pressure changes per unit of intra-band volume change. Intra-band volume changes arise as a result of slight leakage, tissue changes, etc.

Several experiments, as reported below, were conducted to determine the relationship between: (1) changes in magnitude of the band contact area or diameter vs. intra-band pressure (i.e., pressure in the balloon section); and (2) changes in fluid volume in the balloon section vs. the corresponding changes in intra-band pressure (i.e., balloon pressure). The intra-band pressure (P_{intra-band}) is a superposition of the pressures generated by both the contact pressure between the stomach tissue and the band, and the balloon inflation pressure which is the pressure it takes to inflate the balloon portion of the gastric band. There may be other factors that influence the intra-band pressure, such as intra-abdominal pressure. However, the main factors contributing to the intra-band pressure are the contact pressure between the stomach tissue and the band, and the pressure it takes to inflate the balloon.

Several other terms used herein require definition. The term “intra-luminal pressure” (Pᵢₙₜᵣₐ₋ₗᵤₘᵢₙₐₗ) is the pressure inside the lumen (esophagus or stomach) that is at least in part generated by the force of the lap band on the tissue it surrounds (also known as P_{contact} or contact pressure at the balloon-tissue interface). The “balloon inflation pressure” (P_{balloon}) is the pressure required to inflate the lap band balloon when no tissue is encircled (i.e., unconstrained). Under most conditions the intra-luminal pressure and the contact pressure are believed to be of similar magnitude in a static condition. Thus

P_{intra-band}=P_{balloon}+Pᵢₙₜᵣₐ₋ₗᵤₘᵢₙₐₗ

Further, the “pressure-volume compliance” (P-V_{compliance}) as used herein is the slope of the pressure-volume curve and it indicates the change in pressure over a unit change in volume. Thus,

$$slope ( P - {V}_{compliance} ) = \frac{{P}_{2} - {P}_{1}}{{V}_{2} - {V}_{1}} ( \frac{mmHG}{mL} )$$

where P₁ and P₂ are pressure measurements in mmHg and V₁ and V₂ are corresponding unit fluid volume measurements in mL. For example, for a given bladder assembly used with a lap band, the lap band balloon will have a P-V_{compliance-band} and the bladder assembly will have a P-V_{compliance-bladder}. The P-V_{compliance} of the entire system is:

$$P - {V}_{system - compliance} = \frac{Δ P}{Δ {V}_{band} + Δ {V}_{bladder}}$$

To calculate the P-V_{bladder}:

$$P - {V}_{bladder} = \frac{Δ P}{Δ {V}_{system} - Δ {V}_{band}}$$

The ΔV_{system} is the volume of fluid in the system which can include the balloon, bladder, fill port, and associated tubing (and a flow restrictor if used). Under resting/steady-state conditions:

ΔP_{system}=ΔP_{band}=ΔP_{bladder}

### Experiment No. 1

An in vitro model was constructed to show that a bladder could transfer fluid to or from an expandable balloon on a gastric band in response to controlled changes in the size of the stomach tissue encircled by the balloon. To simulate the changes in volume of the encircled stomach tissue/stoma, an aluminum mandrel with varying diameter from 20 mm to 8 mm was fabricated. Each diameter segment was about 25 mm in length along the mandrel. At the end of the 8 mm diameter segment, the mandrel diameter increased to 25 mm, large enough to be held with a pair of soft jaw clamps that were then secured to a stand at a height such that the subject mandrel diameter segment was just above another soft jaw clamp positioned lower on the same stand. A Realize Band® (Ref #RLZB22 made by Ethicon Endo-Surgery, Inc., a Johnson & Johnson company) was slid over the subject mandrel segment such that the band encircled the mandrel. Part of the band where the silicone tubing was connected laid on top of the lower clamp. The reference inlet of a manometer was also attached to the lower soft jaw clamp. A 10 cc syringe was attached to a 3-way stopcock. A 22 gauge Huber tip needle was connected to the stopcock port directly across from the syringe. The pressure reading inlet of the manometer was attached to the side port of the 3-way stopcock and was held in place with a vice. Finally, the Huber tip needle was used to puncture the access port of the Realize Band® system.

The Realize Band® was then placed around the 20 mm diameter segment of the mandrel and the band was supported by the lower soft clamp. A vacuum was drawn with the 10 cc syringe to remove as much air inside the balloon of the band as possible. Water was slowly injected into the access port of the reservoir until the intra-band pressure reached about 30 mmHg. The valve of the three-way stopcock to the syringe port was closed and the intra-band pressure was recorded after the system had reached a steady state. The Realize Band® was moved from the 20 mm diameter segment to the 18 mm diameter segment of the mandrel and the mandrel was lowered so that the 18 mm diameter segment was at the same height as the 20 mm diameter segment had been. The intra-band pressure was recorded after the system had reached a steady state. The steps above were repeated for both mandrel diameter segments of 16 mm and 14 mm.

By varying the mandrel diameter that was encircled by the Realize Band®, the change in stomach tissue volume/stoma diameter was simulated in an in vitro model. The experiment showed that intra-band pressure dropped significantly when the mandrel diameter that was encircled by the band decreased, as shown FIG. 10. Just as Rauth, et al. had hypothesized, the intra-band pressure drop could be related to the decreasing volume of stomach contained within the band.

In addition to Rauth, et al.'s explanation of patients feeling the loosening of the band in between adjustments, Dixon, et al. documented some leakage of saline out of the band over time. Also, others suggested that trapped air inside the band may dissolve or dissipate over time. Both saline leakage and air dissolution would result in a decrease in intra-band volume and hence a decrease in intra-band pressure.

### Experiment No. 2

The Realize Band® was placed over and encircled the 20 mm diameter segment of the mandrel. Part of the band was supported by the lower soft clamp. A vacuum was drawn using the 10 cc syringe to remove as much air as possible from inside the expandable balloon section of the band. The balloon section of the band was next inflated with water in 0.5 mL increments for a total of 9 mL. The intra-band pressure was recorded per each increment increase. The balloon section of the band was next deflated in 0.5 mL decrements and the intra-band pressure was recorded per each decrement and the intra-band pressure was recorded per each decrement.

To demonstrate that intra-band volume change can affect intra-band pressure, the in vitro model described above was used to characterize the pressure-volume relationship of the Realize Band®.

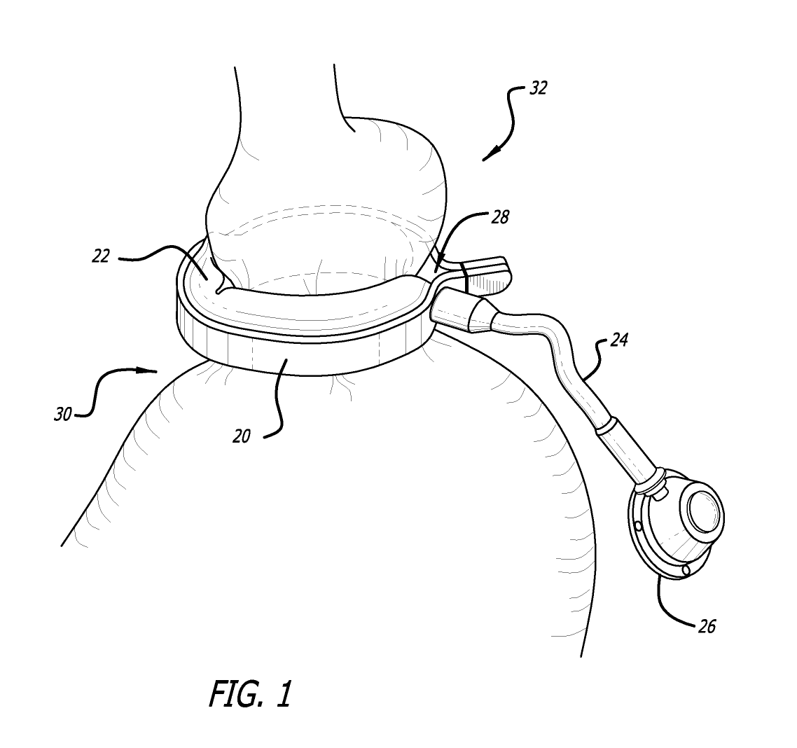

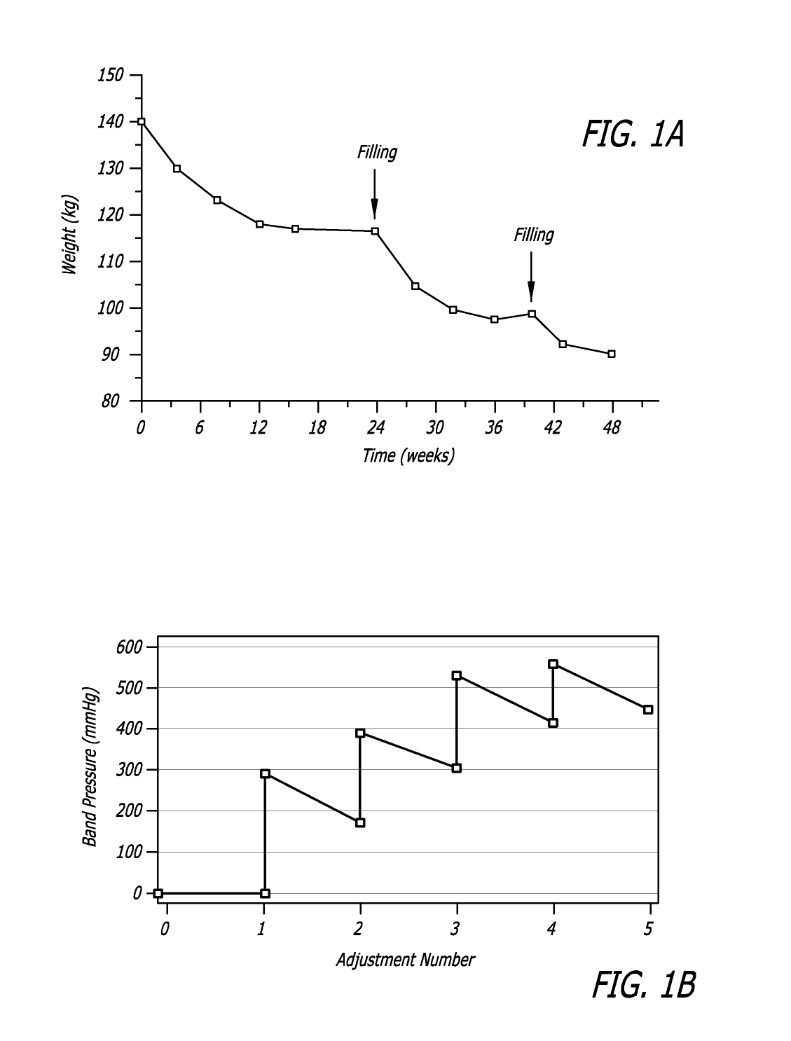

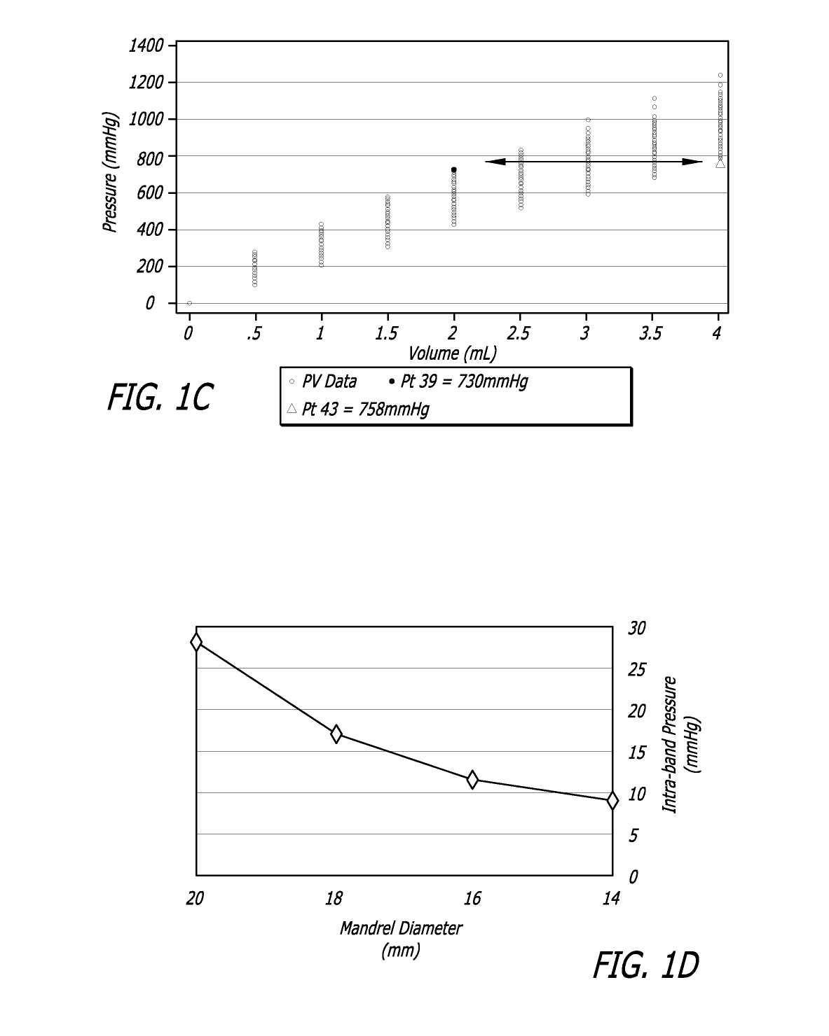

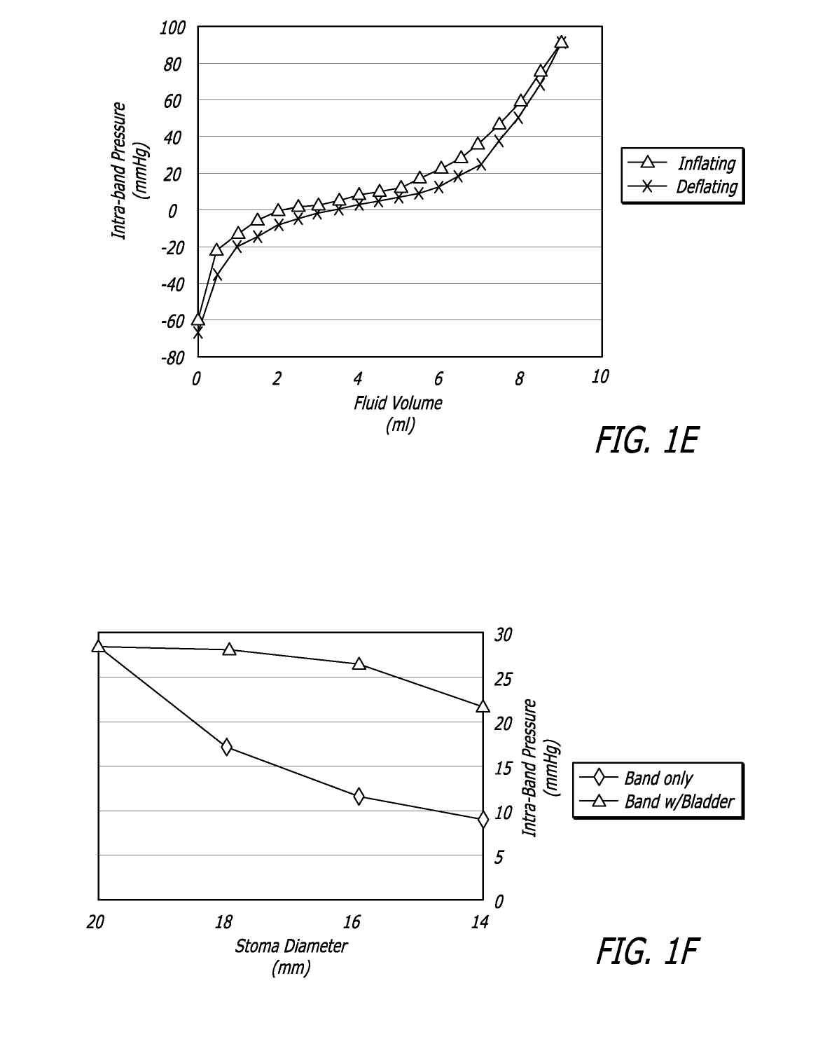

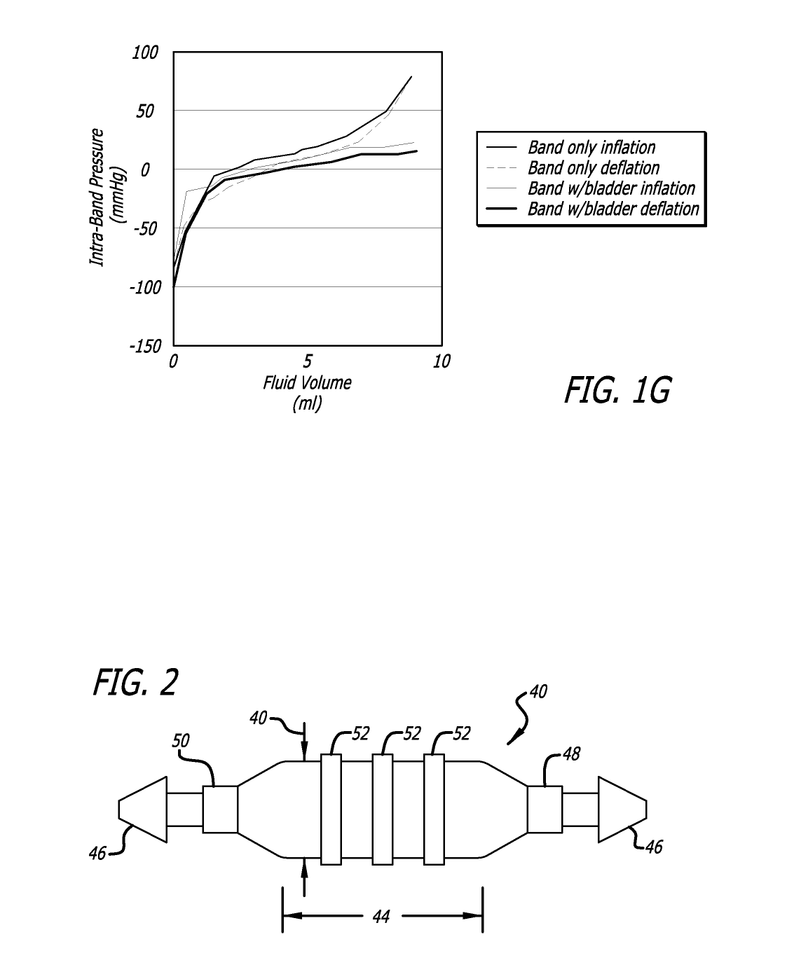

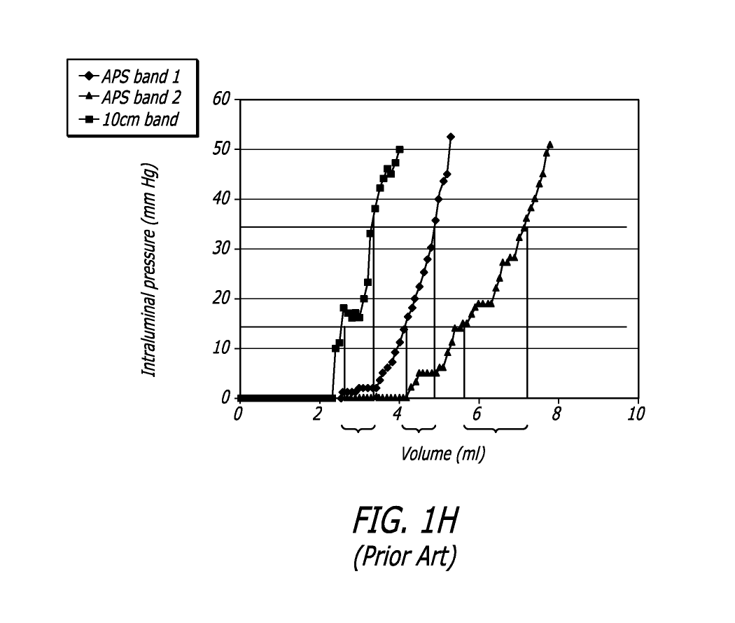

This experiment showed that the intra-band pressure increased with an increase in volume and decreased with a decrease in volume of the expandable balloon. Furthermore, the data showed that the rate of pressure change for a given change in fluid volume increased significantly as the intra-band volume reached its full capacity, which has important clinical implications discussed in detail below. The intra-band pressure and volume curves are shown in FIG. 1E.

The two experiments demonstrated in vitro that both change in stomach tissue volume and change in intra-band fluid volume could affect the intra-band pressure. However, the exact mechanism behind the feeling of band loosening in between adjustments may not be clear. What is clear though is that the addition of small amounts of fluid into the band as is done during the majority of the band adjustments can bring back the feeling of restriction and satiety to the patients.

### Experiment No. 3

In this experiment, a bladder or fluid reservoir was incorporated between the Realize gastric band and a standard fluid infusion port. The bladder was filled with a fluid and was in fluid communication with the infusion port and the balloon portion of the gastric band. In this experiment the bladder had a lower compliance (however, the bladder compliance need only be greater than zero and less than infinity) than the balloon portion of the gastric band, therefore the bladder will fill the gastric band as the inner diameter of the band is reduced. The in vitro experiments described in Experiment 1 were repeated and measurements were taken of the intra-band pressure both with and without the bladder in the system. The data is shown in FIG. 1F. The data shows that the bladder maintained the intra-band pressure over a wide range of encircled tissue volume change as it was simulated by varying (reducing) the mandrel diameter. As the mandrel diameter decreased from 20 mm to 14 mm, the intra-band pressure dropped only 6.5 mmHg (23%) in the system with the bladder vs. a drop of 19 mmHg (68%) in the system without the bladder.

### Experiment No. 4

In this experiment, it was demonstrated that the intra-band pressure could be maintained when the bladder was connected in between the Realize gastric band and the fluid infusion port. In this experiment, a vacuum was drawn to remove as much air from inside the balloon portion of the gastric band as possible. Thereafter, the balloon portion of the gastric band was inflated with water in 0.5 mL increments for a total of 9 mL. The intra-band pressure was recorded at each increment. Thereafter, the balloon portion of the gastric band was deflated in 0.5 mL decrements and the intra-band pressure was recorded at each decrement. As demonstrated by the data, the bladder was able to change the pressure/volume (P/V) characteristics of the gastric band assembly (i.e., the gastric band plus bladder configuration). As can be seen in FIG. 1G, the slope of the P/V curve of the gastric band with the bladder is much flatter than that of the slope of the P/V curve of the gastric band without the bladder in the system, especially in the 6 to 9 mL volume range. The separation is even more pronounced when the intra-band pressure exceeded 10 mmHg.

Based on the experiments above, a bladder could be added to existing gastric bands. Such a bladder would better maintain the intra-band pressure over a wider range of intra-band fluid volume change or encircled tissue volume or tissue-band loading change. By preventing the intra-band pressure from dropping or rising appreciably, patients would be maintained at a pressure and/or stoma size set by the physician longer, thus reducing the number of adjustments necessary or even potentially eliminating adjustments altogether.

This novel bladder is a passive system having a specific predetermined pressure-volume relationship inherent to the system. Based on physiological and clinical observations, the bladder disclosed herein is expected to work in the intra-band pressure range between −40 and +100 mmHg for certain types of commercially available gastric bands (e.g., Realize Band®), but for some gastric or lap bands, the pressure range could be between −40 mmHg and +180 mmHg (e.g., Lap-Band AP-S and AP-L). The intra-luminal and intra-band pressure variations are less severe over a wide range of fluid volume changes with the bladders in the gastric band assembly than in a gastric band assembly without the bladders, i.e., with the gastric band only.

As shown in FIG. 1, a typical prior art gastric band assembly 20 includes an expandable or inflatable balloon section 22 that is connected to tubing 24 in fluid communication with a port 26. The band 20 forms a restriction or stoma 28 so that the stomach 30 has pouch 32 formed above the band. The bladder is incorporated into the gastric band assembly 20.

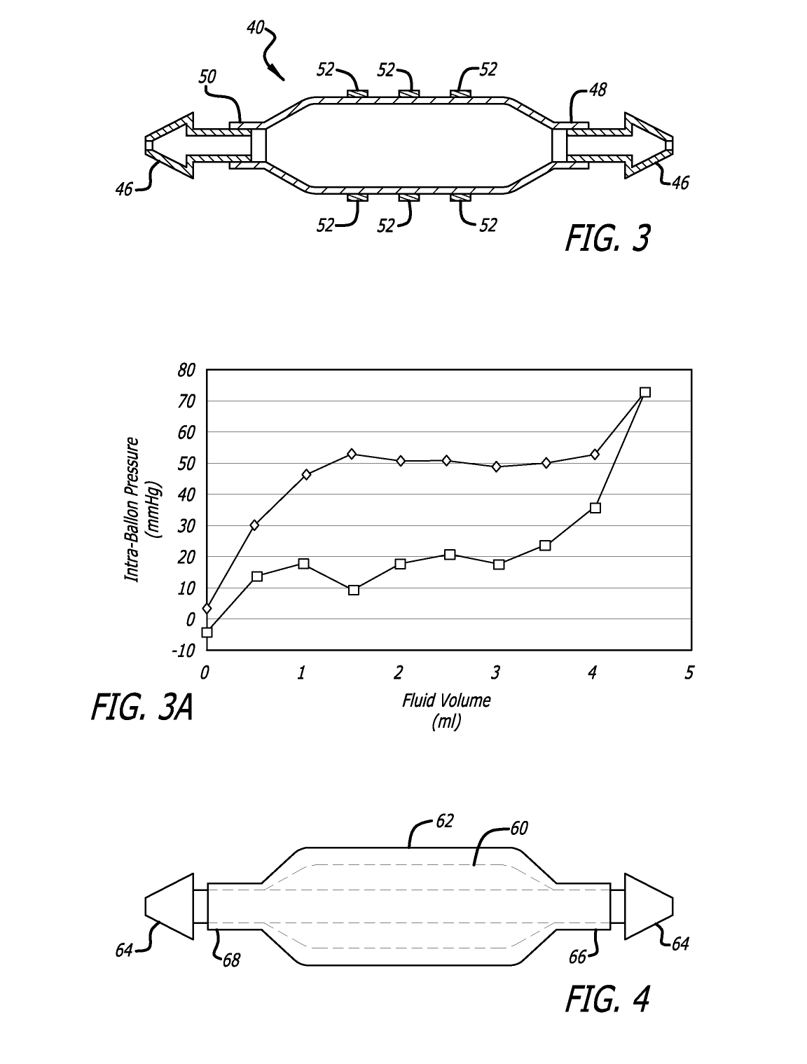

In one embodiment, as shown in FIGS. 2 and 3, a bladder 40 has an outside diameter 42 of no greater than about 15 mm and a length 44 of about 14.0 cm. Importantly, the bladder 40 can take on many different shapes and dimensions. For example, the bladder can have any shape (elongated, tubular, cylindrical, toroidal, annular, and the like), and it can be configured to receive from 0 to 14 mL or more of fluid. The bladder is formed from an elastic material such as polyethelene, silicone rubber, urethane, ePTFE, nylon, stainless steel, titanium, nitinol, cobalt chromium, platinum, and similar materials approved for implanting an in humans. A barbed fitting 46 is attached to the bladder's infusion lumen 48 and discharge lumen 50. Three elastomeric bands 50 are positioned on the outer surface of the bladder with a spacing of about 7 mm between the bands. The bands are made out of synthetic polyisoprene (HT-360 by Apex Medical Technologies) and are highly elastic. In this embodiment, the bladder is substantially inelastic. The bands have an inside diameter of about 5.7 mm, width of about 4.57 mm, and a wall thickness of 0.127 to 0.1651 mm. In this embodiment, the bladder 40 can be incorporated into any typical gastric banding assembly such as that shown in FIG. 1. The bladder 40 would be connected to tubing 24 shown in FIG. 1 by inserting the luer fittings 46 in the tubing so that the bladder 40 was in line with the tubing 24 situated between the port 26 and the balloon 22. The infusion lumen 48 of the bladder 40 is inserted into the tubing 24 toward the port 26, while the discharge lumen 50 of the bladder 40 is inserted into the tubing 24 in the direction of the balloon 22. The bladder 40 can be inserted into any commercially available gastric banding assembly having at least an expandable balloon portion, while it is not necessary to include the port as described.

The bladder can be characterized as an expandable waterproof container with a defined pressure-volume relationship that, when hooked up to a balloon portion of a gastric band, alters the pressure volume relationship of the balloon system, making its compliance curve flatter. The bladder can be elastic, pseudo-elastic, or exhibit other characteristics, but it is biased to return to a resting low volume state from a stretched or filled state. The bladder can be an expandable balloon or bellows, made of plastic, metal, or rubber (or a combination of these materials). It is impermeable to saline, contrast media, and similar materials, although it may leak slightly over time. The bladder is made of any biocompatible material and is MRI compatible. The bladder is durable, reliable and fatigue resistant. If the bladder ruptures, the system is still functional and can still be adjusted by adding and removing saline or other fluid. The present invention bladder can be located anywhere in the system, even within the balloon portion of the gastric band. The bladder can be located in the connecting tubing between the balloon portion of the gastric band and the fill port, within the fill port, or as a separate component of the system. The bladder may or may not have a protective shell or housing surrounding the bladder. Such a shell or housing provides protection to the bladder and also acts as a limit to the expansion or distension of the bladder. When the bladder is filled with fluid, any further filling above a certain volume will result in a significant rise in pressure. The surgeon will be able to feel this pressure through the syringe used to fill the bladder. This acts as a tactile set point for the surgeon. For example, the surgeon may fill the band until this significant rise in pressure is felt, and then remove some fluid, perhaps 1 cc, so that the bladder not only has room to contract, but also to expand if the balloon portion of the gastric band feels an increased squeeze or pressure.

The embodiment of the bladder 40 disclosed in FIGS. 2 and 3 was tested to establish a intra-balloon pressure vs. fluid volume chart as seen in FIG. 3A. The test results showed that there were two pressure plateaus where the intra-bladder pressure was maintained over a range of intra-bladder fluid volume. During bladder 40 inflation (the upper curve), a pressure plateau around 50 mmHg was formed when fluid volume increased from 1.5 mL to 4 mL, a range of 2.5 mL. During bladder deflation (the lower curve), a second pressure plateau around 20 mmHg was formed when fluid volume decreased from 3.5 mL to 1 mL, a range of 2.5 mL. This phenomenon was not expected since the polyethylene bladder alone (without the bands 52) did not exhibit similar pressure/volume characteristics. It is the combination of the bands 52 elasticity and the unfolding/folding of the non-elastic bladder that created this pressure/volume curve. Consequently, different plateaus are achieved with different band elasticity and bladder folding geometries.

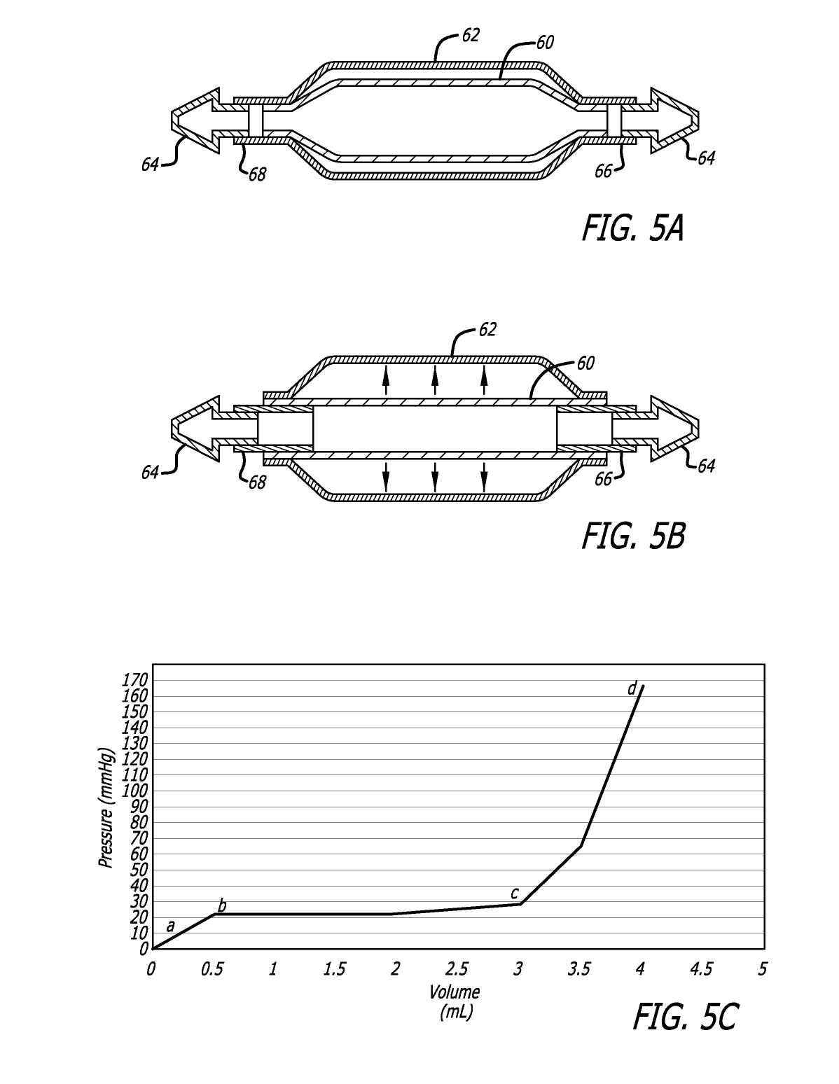

In another embodiment, as shown in FIGS. 4 and 5A and 5B, a bladder 60 having an outside diameter not to exceed 15 mm, is encased in a hard plastic housing 62. Barbed fittings 64 are attached to the infusion lumen 66 and discharge lumen 68 of the housing 62. In this embodiment, the bladder is formed of an elastomeric material which could be in the form of a tube. The bladder 60 could be made out of any number of elastomers from which specific and desired pressure-volume compliance curves can be controlled by the dimensions of the elastomeric tubing, and the type of polymer used in the tubing material. Importantly, bladder 60 is housed within housing 62 so that as the bladder is inflated with a fluid through the infusion lumen 66, the bladder 60 will expand until it contacts the inner walls of housing 62. The housing 62 isolates the bladder from surrounding tissue and limits the total volume that the bladder can expand. Further, the housing 62 will alter the pressure-volume compliance curve of the bladder as seen below in Table 6. As with the other embodiments disclosed herein, bladder 60 and housing 62 can be incorporated into any gastric banding system such as the one shown in FIG. 1. Further, the housing is fluid tight and acts as a fail-safe mechanism in the event the bladder 60 leaks, and the balloon 22 associated with the gastric band 20 will still function as if the bladder 60 was not present in the system. In other words, fluid can still be injected through port 26 (FIG. 1) and tubing 24, and through the bladder 60 which is FIG. 5C, before bladder 60 is inflated, pressure rises as the volume increases (graph segment a-b). As the bladder is inflated, the pressure is held constant (at about 20 mmHg) even though the volume inside the bladder 60 increases from about 0.6 mL to about 3.0 mL (graph segment b-c). Once the bladder 60 is completely full and pressing against the inside wall of housing 62, the pressure rises dramatically as the volume increases (graph segment c-d).

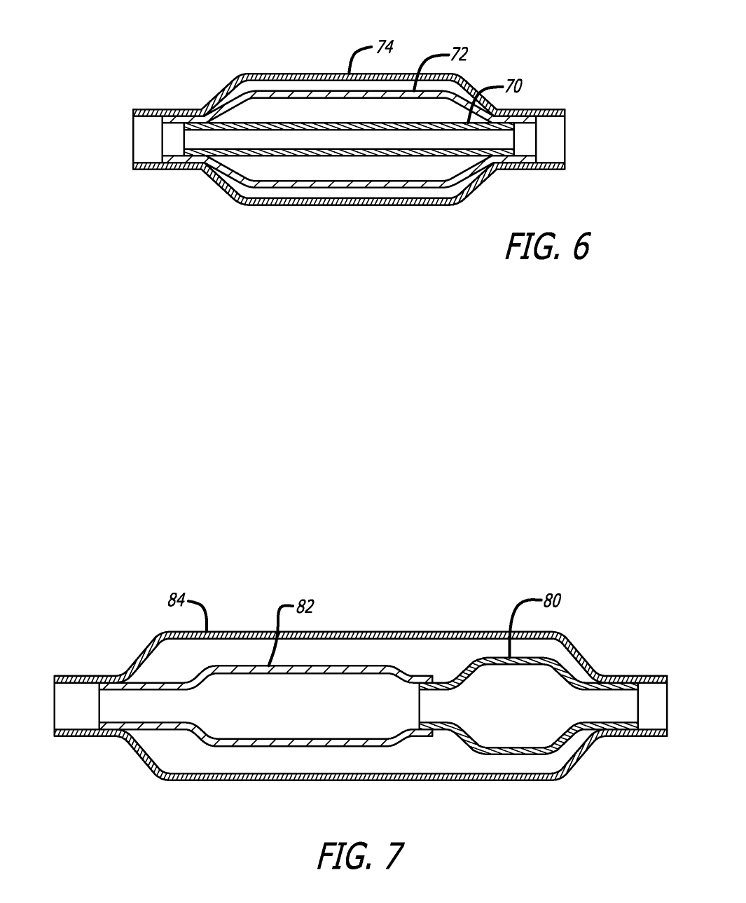

In an alternative embodiment, as shown in FIG. 6, more than one bladder can be used in the system in order to create multiple pressure-volume characteristics. For example, in the FIG. 6 embodiment, a first bladder 70 and a second bladder 72 both are housed in a hard plastic housing 74. The barbed fittings from previous embodiments are not shown for clarity. In this embodiment, the compliance of first bladder 70 is substantially higher than the compliance of the second bladder. As fluid is injected into the first bladder 70, it will easily expand until it comes into contact with the second bladder. Since the second bladder has less elasticity than the first bladder, it will begin to expand well after the first bladder is expanded. As the volume continues to increase, the second bladder also will expand until both the first bladder 70 and the second bladder 72 can no longer expand because the second bladder contacts housing 74. In this embodiment, the second bladder 72 will have a higher constant pressure plateau than the first bladder 70.

In a similar embodiment to that shown in FIG. 6, two bladders can be connected in series within a single housing to effect two different constant pressure plateaus. As shown in FIG. 7, first bladder 80 has a higher elasticity than second bladder 82. Both bladders are encased in housing 74 and, as with FIG. 6, the luer fittings have been omitted for clarity. As fluid is added to the system, first bladder 80 is designed to fully expand into contact with housing 84 before the second bladder 82 begins to expand. After first bladder 80 is fully expanded, second bladder 82 will expand as more fluid is injected into the system until second bladder 82 contacts housing 84. The pressure/volume curves for this embodiment are expected to be similar to that shown in Table 4. Both embodiments shown in FIGS. 6 and 7 can be incorporated into an existing gastric banding system such as the one shown in FIG. 1.

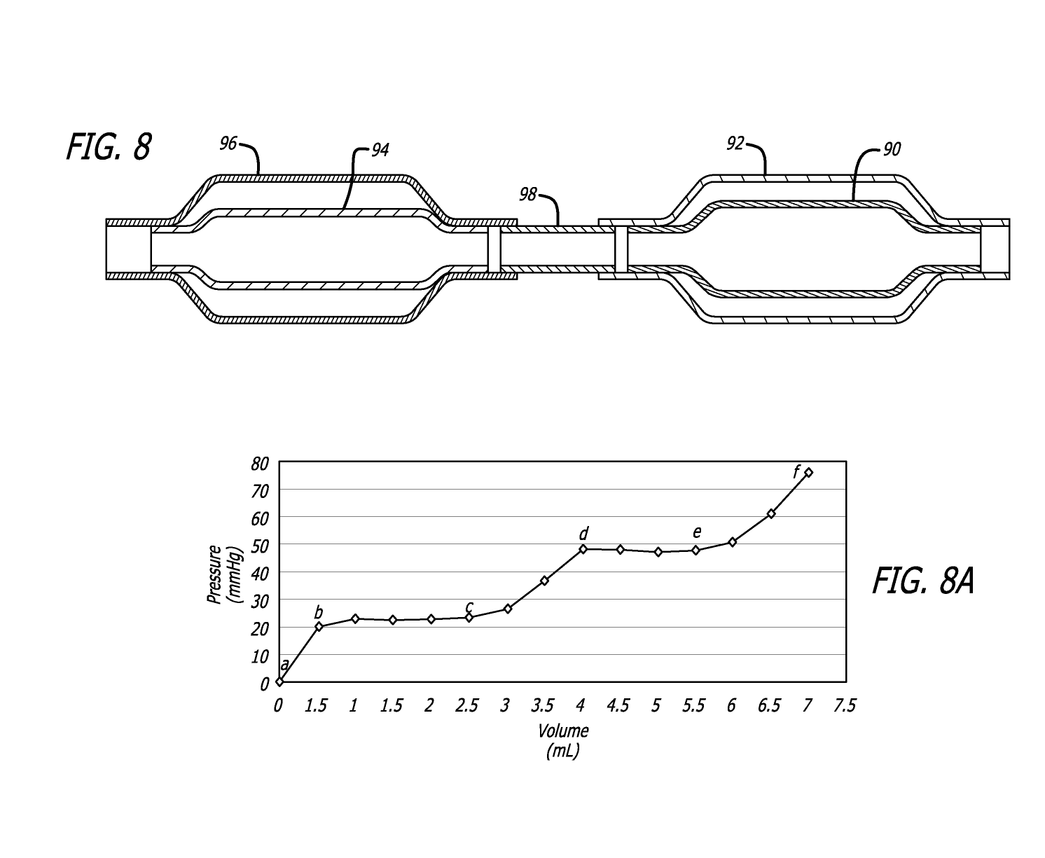

In another embodiment, as shown in FIG. 8, a first and second bladder are arranged serially or in line in separate housings. In this embodiment, first bladder 90 is encased within hard plastic first housing 92 and is in serial fluid communication with second bladder 94 which is encased in hard plastic second housing 96. In this embodiment, first bladder 90 is more elastic than is second bladder 94, so that as the fluid is injected into first bladder 90 it will expand until it contacts the inner surface of first housing 92, before second bladder 94 begins to expand. A tubing 98 is used to connect the housings. As with the other embodiments, the luer fittings have been omitted for clarity. In this embodiment, second bladder 94 has a higher constant pressure plateau than the first bladder 90. Before first bladder 90 begins to inflate, the pressure is held constant (about 20 mmHg) even though the volume increases (from 0.5 to 2.5 mL) as can be seen in FIG. 8A. in the graph segment b-c. Once first bladder 90 fills the entire cavity of the first housing 92, the pressure rises as volume increases, as shown in graph segment c-d. As the volume continues to increase, second bladder 94 will start to inflate and the pressure is once again constant, albeit at a higher pressure level (about 50 mmHg in graph segment d-e) than the constant pressure level exhibited by the filling of first bladder 90. As the second bladder 94 fills the entire cavity of second housing 96, the pressure again rises as the volume increases as shown in graph segment e-f. This embodiment also can be incorporated into any gastric banding system, such as that shown in FIG. 1.

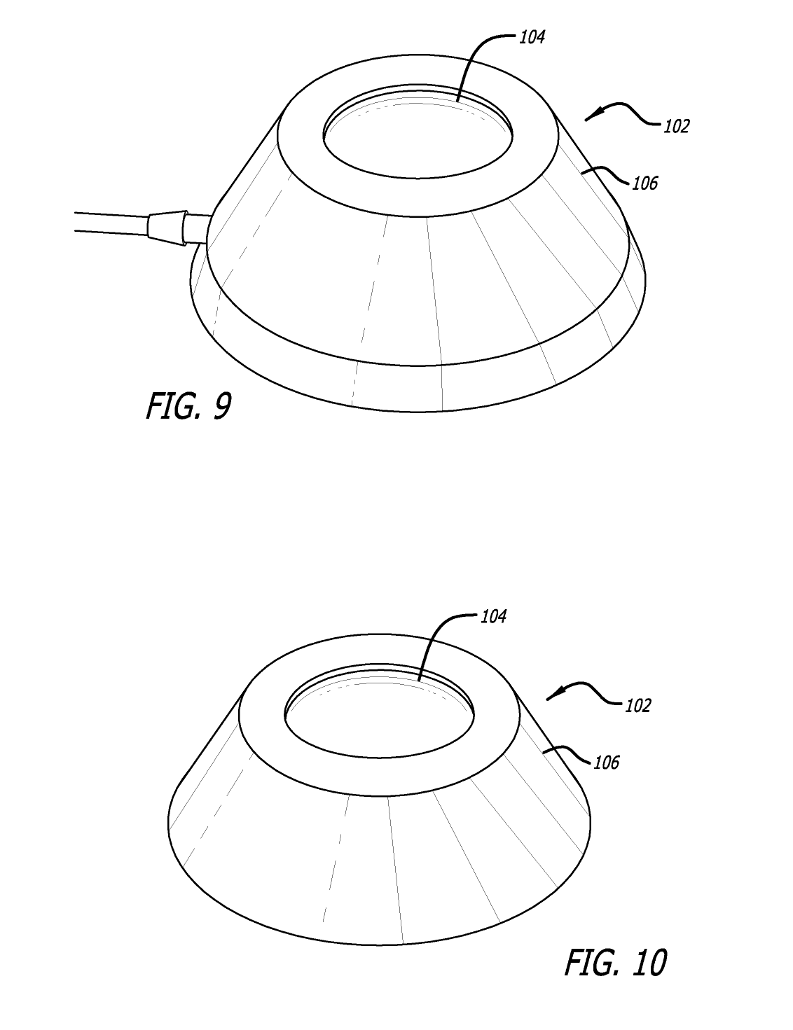

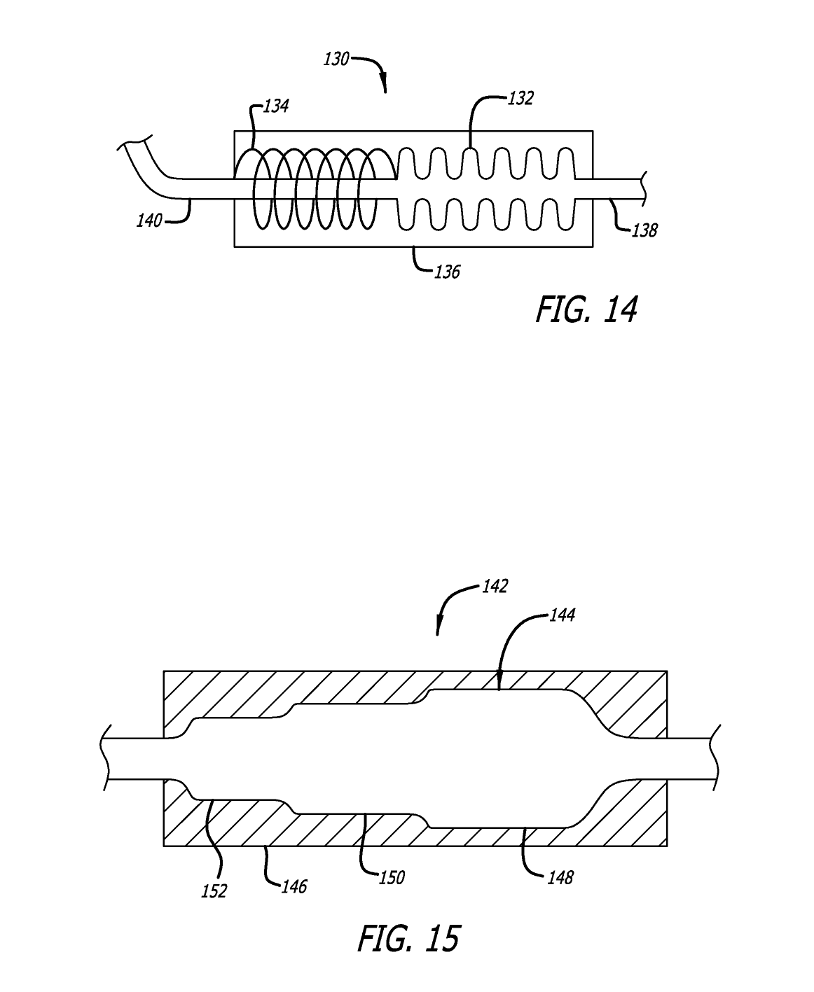

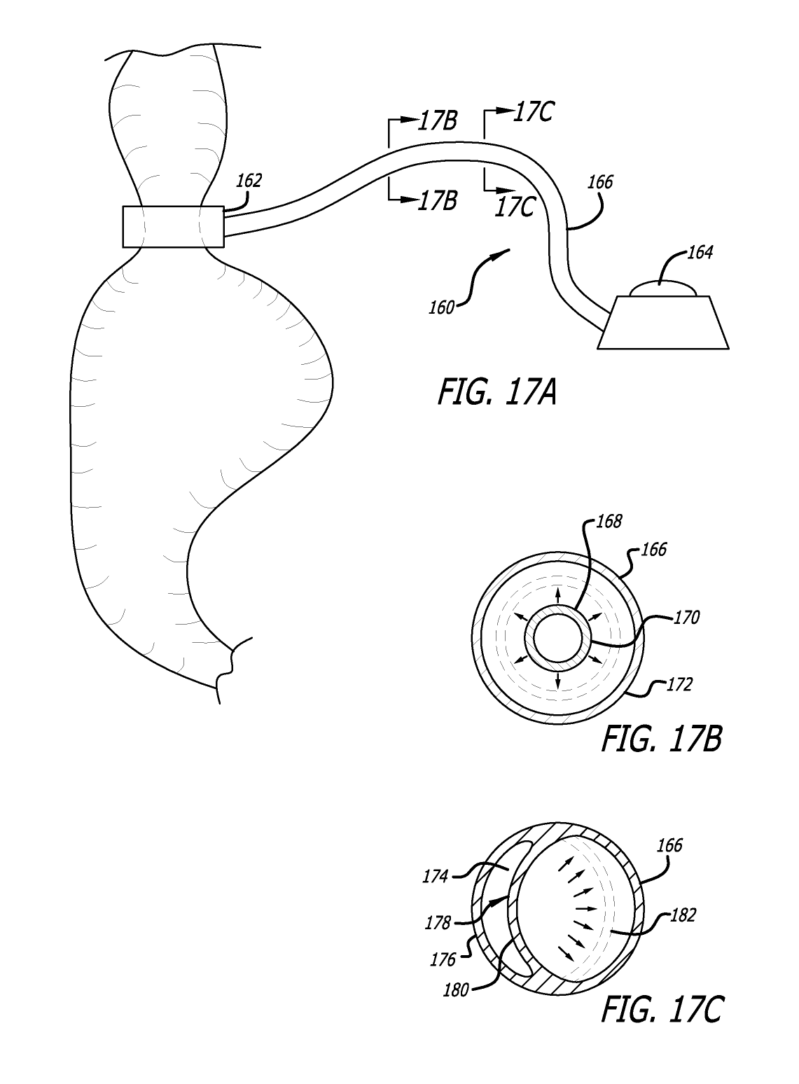

In another embodiment, as shown in FIGS. 9-13, an injection port bladder assembly 100 houses an expandable bladder and is designed to be mounted toward the surface of the skin so that fluid can be injected with a needle to replenish fluids in the system. The injection port bladder assembly 100 is comprised of a housing 102 made of a hard shell plastic, such as polysulfone or titanium, or a combination of both. Housing 102 can be molded or machined. The housing includes a septum 104 which is a self-sealing silicone rubber seal positioned in the top cavity 106 of housing 102. Fluid is injected into the housing by puncturing septum 104 with a needle, and after fluid is injected into the housing, the needle is removed and the septum 104 automatically seals to prevent leakage. The top cavity 106 mates with bottom cavity 108 and the two halves of the housing 102 are sealed together in a known manner. The top and bottom cavity 108 contains expandable bladder 110 in the form of an annular, circular or toroidal configuration. In this embodiment, the bladder 110 can have other configurations and still reside in cavity 108. For example, the bladder could be formed of coaxial tubing similar to that shown in FIGS. 17A and 17B, it could have a septum (FIGS. 17A and 17C), it could have a bellows configuration (FIG. 14), or it could be donut, disk or irregular-shaped, as long as the bladder fits in cavity 108. More broadly, bladder 110 can have any shape that allows it to flex or deform elastically thereby imparting pressure on the fluid within the system consistent with the compliance curves disclosed herein.

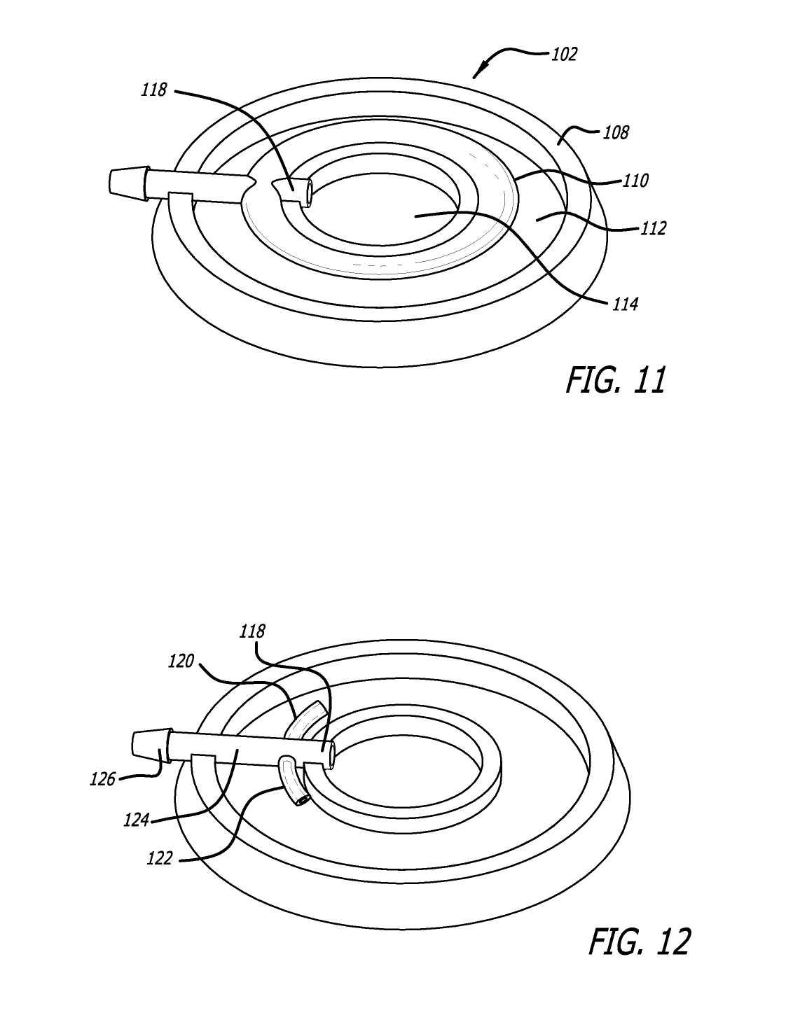

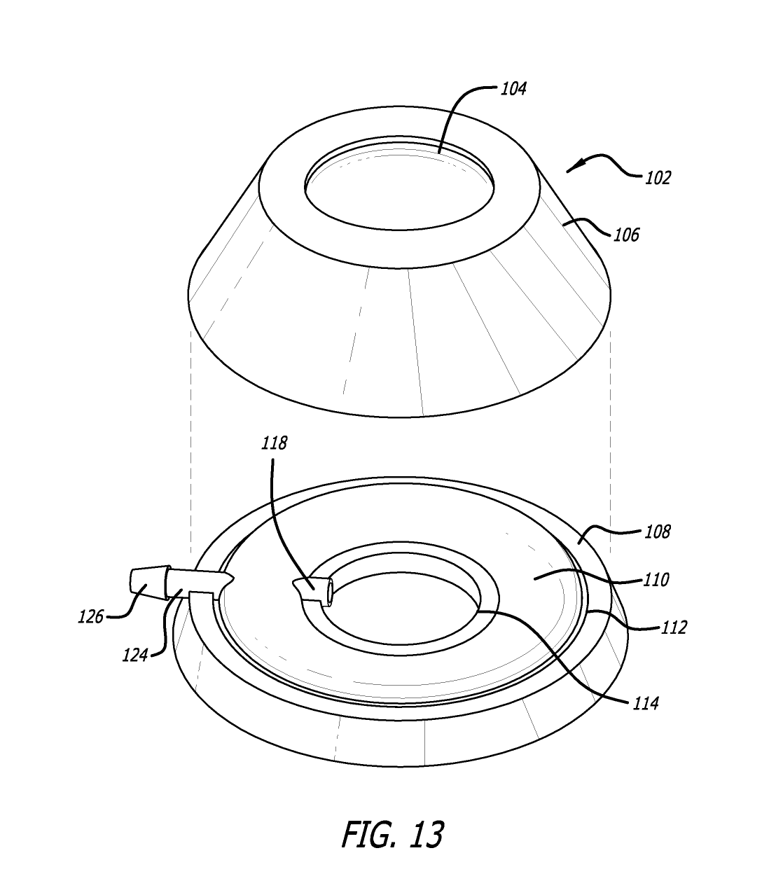

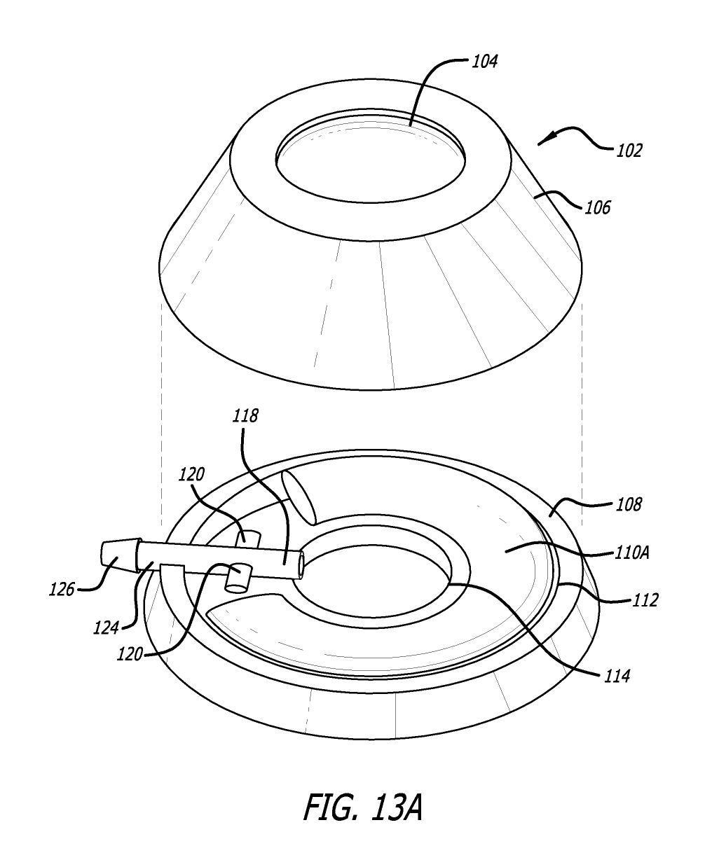

The bladder is mounted in the cavity 108 along a toroidal surface 112 (or within a toroidal chamber or volume). Bladder 110 is shown in FIG. 11 in a deflated configuration and in FIG. 13 in an inflated configuration. Fluid flows into bladder 110 via fluid chamber 114. A cross connector 116 is attached to the bottom cavity 108 and has four arms. First arm 118 extends into fluid chamber 114 and provides a flow pathway from the fluid chamber into the second arm 120 and the third arm 122. Bladder 110 is connected to the second arm 120 and third arm 122 so that fluid from the fluid chamber 114 flows through first arm 118 and second arm 120 and third arm 122 in order to allow fluid flow into and out of bladder 110. A fourth arm 124 is in fluid communication with the first arm 118, second arm 120, and third arm 122. Fluid flows from the fourth arm 124 through tubing (not shown) to the gastric band and into the balloon portion of the gastric band. The fourth arm 124 has a barbed fitting so that the tubing can be securely attached to the fourth arm.

Still with reference to FIGS. 9-13, the injection port bladder assembly 100 is attached to any conventional gastric banding system such as the one shown in FIG. 1. In this embodiment, the port 26 and tubing 24 shown in FIG. 1 is unnecessary, since the injection port bladder assembly 100 replaces the port 26. In further keeping with the invention, the injection port bladder assembly is attached to a gastric band and a conventional syringe is used to inject fluid through septum 104 in order to fill fluid chamber 114. As fluid flows into the fluid chamber, the fluid flows through the cross-connector 116 and fills bladder 110 so that it expands against the toroidal surface 112. Expansion of the bladder is limited against the constraint of the wall of the toroid surface 112 (see FIG. 13). As fluid flows into bladder 110, fluid also flows through cross-connector 116, including through fourth arm 124 and tubing (now shown) to the gastric band, and more particularly into the balloon portion of the gastric band. As set forth above, the bladder 110 and the balloon portion 22 of the gastric band 20 automatically and continuously equalize pressure in the system in response to changes in the restriction surrounded by the balloon portion of the gastric band. Alternatively, as shown in FIG. 13A, the injection port bladder assembly 100 is similar to that shown in FIGS. 9-13. In this embodiment, fluid does not flow into bladder 110a, rather the bladder 110a is filled with a compressible material such as air, foam, micro-bubbles, or a similar compressible material. The bladder 110a is a closed system and prior to injecting fluid into septum 104, the bladder 110a is in an expanded configuration. As fluid is injected into or through septum 104, the fluid fills chamber 114 and flows through first arm 118 and second arms 120 so that the fluid flows around bladder 110a. As the fluid is further injected into the injection port, the fluid compresses bladder 110a which causes the pressure on the fluid to build up so that the pressure on the fluid will flow through fourth arm 124 to the balloon portion of the gastric band. Since the fluid pressure in the injection port bladder assembly 100 is higher than that in the balloon portion of the gastric band, the pressure will automatically and continuously equalize in the system in response to changes in the restriction surrounded by the balloon portion of the gastric band.

Some patients receiving prior art gastric bands may exhibit periods of non-responsiveness so that their weight loss might be sporadic, or in some cases, the patient stops losing weight altogether. The bladder assemblies disclosed herein are particularly useful for these patients because the bladder can be incorporated into gastric bands that already have been implanted. For example, for patients having a Realize Band® with an infusion port to replenish fluid in the balloon portion of the band, bladders of the type disclosed in FIGS. 9-13A can easily be incorporated into the system. The patient is given a local anesthetic so that the infusion port may be removed by a minimally invasive incision. Thereafter, injection port bladder assembly 100 is implanted minimally invasively and attached to the Realize Band® via existing tubing or replacement tubing associated with the bladder assembly 100. After the injection port bladder assembly 100 is attached to the Realize Band®, fluid is injected into the bladder to pressurize the bladder and fluid will automatically flow into the balloon portion of the band. The minimally invasive incision is closed. Thereafter, bladder assembly 100 operates as discussed for FIGS. 9-13A herein in order to maintain the patient's weight loss at an optimal level.

In another embodiment, as shown in FIG. 14, a bladder assembly 130 includes an expandable bellows 132 that can be formed from an expandable material such as silicone rubber or the like. The bellows can be formed of other materials as long as it is expandable or contractible in an accordion fashion. A spring 134, which is optional, is used to generate pressure within the bellows 132. The spring 134 is compressed against a wall of housing 136 and at its other end against the bellows 132, in order to apply a compressive force on the bellows. Housing 136 can be of any material that is biocompatible and protects the bladder assembly 130. Fill tubing 138 is connected to one of bellows 132 for adding or removing fluid to the bellows 132. An infusion tubing 140 is connected to the opposite end of the bellows and is in fluid communication with the gastric band assembly, such as the one shown in FIG. 1. In operation, the bellows 132 is filled with a fluid such as saline which causes the bellows to expand against the compressive force of spring 134. Depending upon the compliance of bellows 132, the spring 134 may not be necessary for a particular system. In this embodiment, the fluid pressure between the bellows and the balloon portion of a gastric band automatically and continuously adjust so that there is no lasting pressure differential between the expandable balloon and the bellows, and in so doing, the pressure in the balloon is maintained even though there are changes in fluid volume in the balloon. Even as the volume of fluid in the balloon portion of the band changes in response to loading changes, the pressure between the bellows and the balloon remains substantially constant and adjusts the amount of fluid in each continuously and automatically in response. This embodiment of the invention, as with the others disclosed herein, eliminate the need for frequent visits to the doctor to have the balloon portion of the gastric band refilled in order to maintain the patient in the adjusted pressure zone.

As shown in FIG. 15, a multi-pressure plateau pressure bladder is disclosed to provide a range of fill volumes that correspond to a range of intra-band pressures. Instead of measuring intra-band pressure to determine how much volume should be put into the balloon portion of a gastric band as typically is done with the prior art devices, this embodiment, as with the others disclosed herein, allow setting intra-band pressure based on the volume of fluid injected into the band. Further, the embodiments of the present invention also provide adjustment of pressure within a predetermined and known range by measuring the volume of fluid injected by the bladder into the balloon portion of the gastric band. This result is achieved without intra-band manometry which is too cumbersome and time-consuming to be widely used. As shown in FIG. 15, a bladder assembly 142 includes a multi-compliant bladder 144 encased in a solid housing 146. The multi-compliant bladder 144 consists of multiple inflatable sections or segments each of which has a different compliance. Thus, as shown in FIG. 15, a first bladder section 148, second bladder section 150, and third bladder section 152 form the multi-compliant bladder 144. The first bladder section has the highest compliance and is the most elastic and as fluid is added to the bladder assembly 142, the first bladder section 148 will expand first. In order to shift the compliance into the higher range of the second bladder section, expansion of the first bladder section 148 must be limited. This can be accomplished by using a rigid, solid housing 146 that will constrain each of the bladder sections as they expand. Thus, as fluid is added to the bladder assembly, the first bladder section 148 will expand until it is limited by solid housing 146, thereby increasing the pressure enough to cause expansion or dilation of second bladder section 150. The solid housing 146 also prevents the first bladder section 148 from rupturing. As fluid continues to flow into the bladder assembly 142, the second bladder section 150 will continue to expand or dilate until it also contacts solid housing 146, whereupon the pressure again will increase so that the third bladder section 152 also will expand.

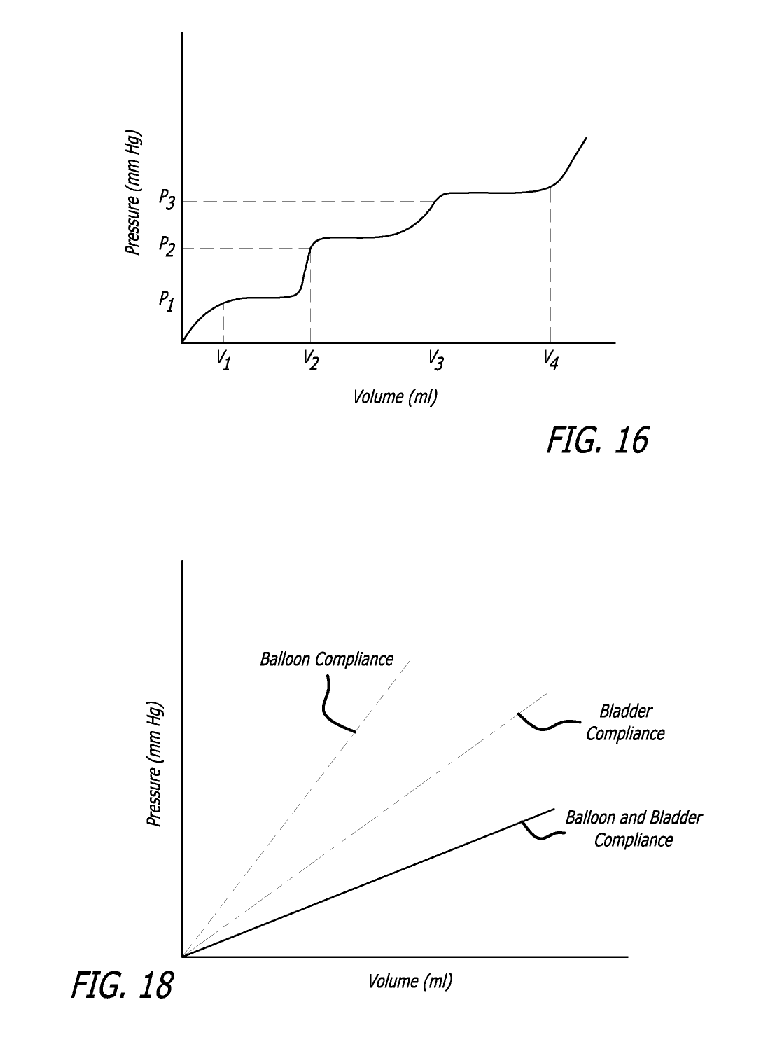

The compliance curves for the embodiment shown in FIG. 15 is shown in FIG. 16. With the use of multi-pressure plateau pressure bladder assembly, a range of fill volumes will correspond to a range of intra-band pressures. Thus, as shown in FIG. 16, for a fill volume between V₁ and V₂, which corresponds to the filling of first bladder section 148, the intra-band pressure (at the balloon's portion of the gastric-band) will be nearly constant at P₁. For a fill volume between V₂ and V₃, which corresponds to the filling of second bladder section 150, the intra-band pressure will be P₂. Likewise, for a volume between V₃ and V₄, the intra-band pressure will be P₃.

In another embodiment, shown in FIGS. 17A-17C, a bladder assembly 160 includes a gastric band 162 and an injection port 164 connected by tubing 166. The tubing 166 is in fluid communication with the gastric band and the balloon portion (not shown) of the gastric band as previously described herein. In this embodiment, some or all of the tubing 166 acts as a bladder. For example, as shown in FIG. 17B, all or a portion of tubing 166 includes a coaxial tubing bladder 168 that extends from the gastric band 162 to the injection port 164. The tubing bladder 168, which is in coaxial alignment with tubing 166, has a first diameter 170 in which there is no fluid flowing through tubing bladder 168. The tubing bladder 168 has a second diameter, that is expanded radially outwardly from fluid being injected into the injection port 164 and flowing into tubing bladder 168. The tubing bladder 168 is formed of an elastic material such as the ones described herein is elastic so that it will expand radially outwardly to second diameter 172. The tubing bladder 168 has a compliance that is lower than the compliance of the balloon portion of the gastric band 162 so that the fluid in tubing bladder 168 is under pressure and will automatically flow into the balloon portion of the gastric band to automatically adjust for patient weight loss as described herein. Similarly, as shown in FIG. 17C, the tubing 166 is separated into two chambers. In this embodiment, bladder 174 is one chamber and it is in fluid communication with the injection port 164 and the balloon portion of the gastric band. The bladder 174 is formed by an outer wall 176 of tubing 166 and a septum 178 that is elastic and is capable of expanding radially outwardly due to fluid pressure within bladder 174. As fluid is injected into injection port 164, the fluid flows into bladder 174 causing the septum 178 to move radially outwardly from it relaxed configuration 180 in the direction of the arrows to its expanded configuration 182. In the expanded configuration, the bladder 174 exerts pressure on the fluid within. The septum 178 is highly elastic and has a lower compliance than the balloon portion of the gastric band, therefore the pressure of the fluid in the bladder 174 will continuously and automatically cause fluid to flow into (or out of) the balloon portion of the gastric band depending upon the changes in the size of the restriction due to the weight gain or the weight loss of the patient.

With respect to the embodiments of the invention disclosed herein, there are a number of different compliance characteristics that may be imparted by the pressure bladder to a gastric banding system. The most appropriate compliance characteristics, both qualitatively and quantitatively, may depend on the compliance characteristics of the gastric band to which the bladder will be made, the desired patient management strategy, and characteristics of the individual patient. Four qualitatively distinct compliance curves are shown in FIGS. 18-21 and described as follows. In FIG. 18, a linearly increasing or decreasing compliance curve is shown, as fluid is injected into the balloon portion of the gastric band, the intra-band pressure rises proportionately. The addition of a compliant bladder to a compliant balloon, overall increases the compliance of the balloon system. After the bladder has been filled with fluid, then for a given change in balloon fluid volume, there is less of an accompanying change in the intra-band pressure (as compared to the balloon system without the bladder). From a clinical standpoint, in the event of fluid leakage from the balloon, an onset of tissue edema, stoma remodeling, etc., there would be less change to the intra-band pressure. Consequently, the patient may stay at or near the physician set pressure and/or set stoma size longer. A linear curve also retains the inherent balloon characteristic of adjustability. Pressure can still be adjusted by adding or removing fluid volume to the system. The slope of the bladder compliance curve has limits. If the balloon system compliance curve is too steep, it will not hold enough fluid volume to meaningfully maintain intra-band pressure. If the bladder system compliance curve is too shallow, it will require too much fluid volume.

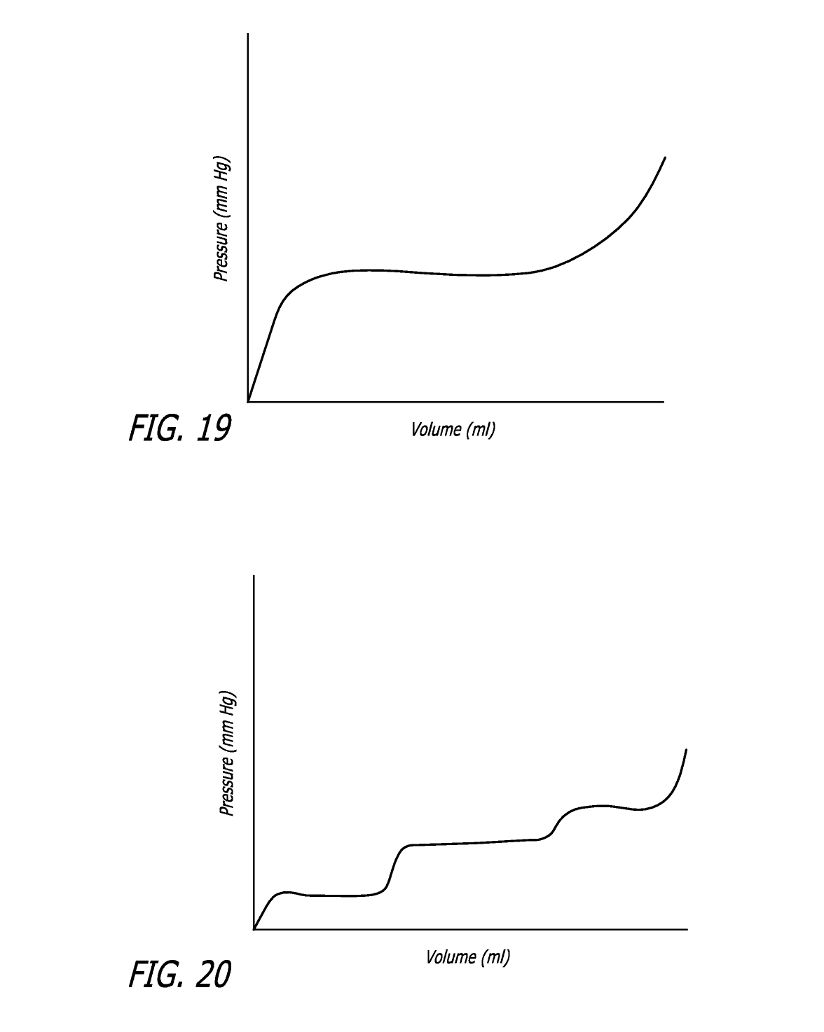

With reference to FIG. 19, a flat or constant pressure compliance curve is shown. In this embodiment, the compliance would keep the intra-band pressure at a substantially constant level over a wide range of volumes. This characteristic may be desirable in maintaining the patient at or near the physician set pressure without adjustments. In this embodiment, the pressure can be set in a specific range for a specific commercially available gastric band. For example, for the Realize gastric band (Johnson & Johnson) the pressure can be set at 40 mmHg up to 150 mmHg. Similarly, for a Lap-Band AP (Allergan), the pressure range may be set somewhat higher, in the range of 40 mmHg up to 180 mmHg.

Referring to FIG. 20, a multi-staged constant pressure compliance curve is shown. The lack of adjustability of some of the embodiments can be overcome with a multi-plateau compliance curve. In this embodiment, pressure can be based on fill volume. Thus, for any particular fill volume, there will be a corresponding constant pressure until a next level of fill volume is added to the bladder system. The embodiment of the bladder assembly shown in FIG. 15 could produce a compliance curve such as that shown in FIG. 20.

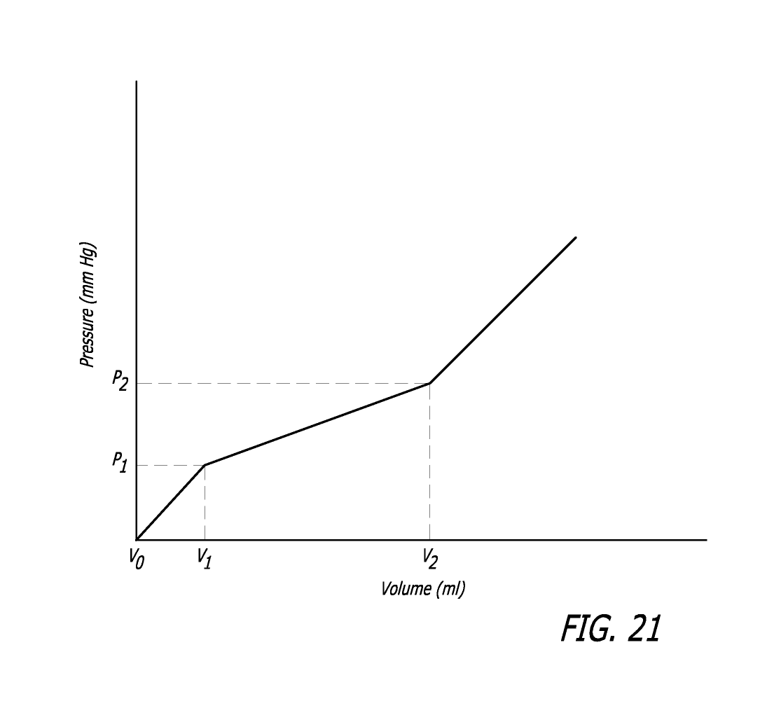

With reference to FIG. 21, a multi-staged linearly increasing compliance curve is shown. In this embodiment, the compliance curves are linearly increasing in staged distinct slopes. In this embodiment, the gastric band would operate between V₁ and V₂. The initial slope, from V₀ to V₁, is steeper in order to reduce the volume of fluid needed to enter the operating zone. The slope in the operating range would be relatively flat, but would allow the surgeon some degree of adjustability. For example, for use with the aforementioned Realize Band®, the P₁ and P₂ intra-band pressures might be in the range from 40 mmHg to 120 mmHg respectively, as long as these intra-band pressures result in an intra-luminal pressure anywhere in the range from 30 mmHg to 150 mmHg.

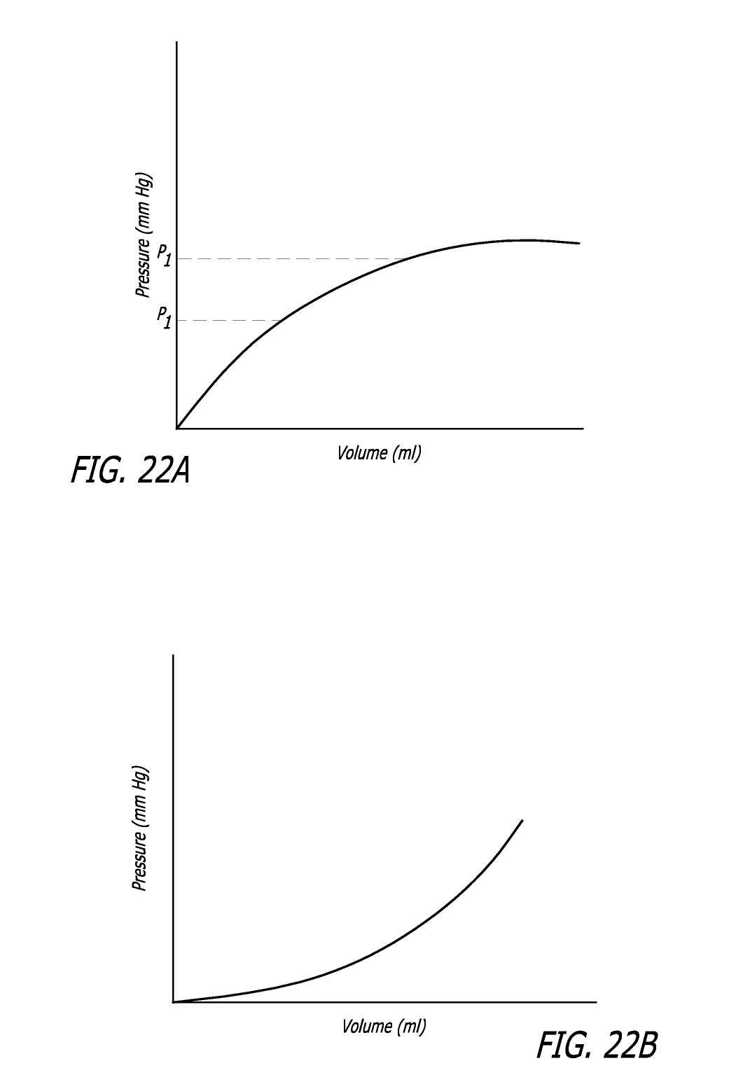

As shown in FIGS. 22A and 22B, logarithmic and exponential compliance curves may be suitable for some patients.

The bladders used herein can be formed from any number of known elastic materials such as silicone rubber, isoprene rubber, latex, or similar materials. As an example, a bladder can be formed by coating silicone rubber on a 0.188 inch outside diameter mandrel to a thickness of about 0.005 inch. Once cured, the silicone rubber coating is removed from the mandrel in the form of a tubing, and can be cut to various lengths in order to form the bladder. As an example, the tubing forming the bladder can range in lengths from 10 mm up to 80 mm, and in one preferred embodiment, is approximately 20-40 mm in length. The tubing can have an outside diameter of approximately 0.125 inch and an inside diameter of 0.0625 inch. The compliance (pressure vs. volume) curve of the bladder can vary depending on a number of factors including in the durometer rating of the silicone rubber, the wall thickness of the tubing forming the bladder, and the shape of the bladder.

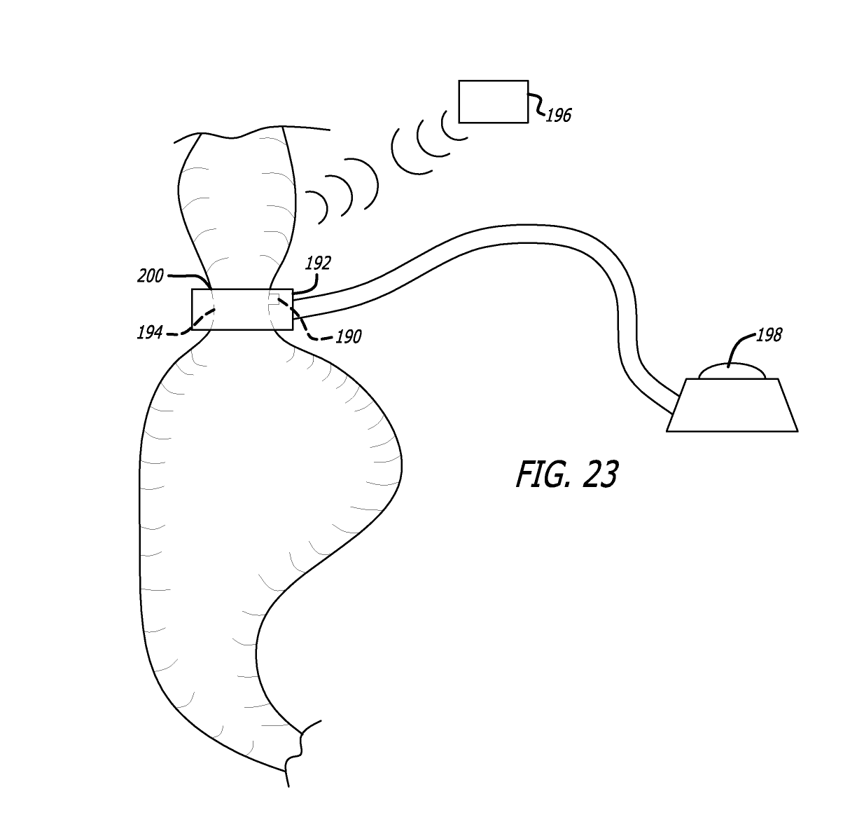

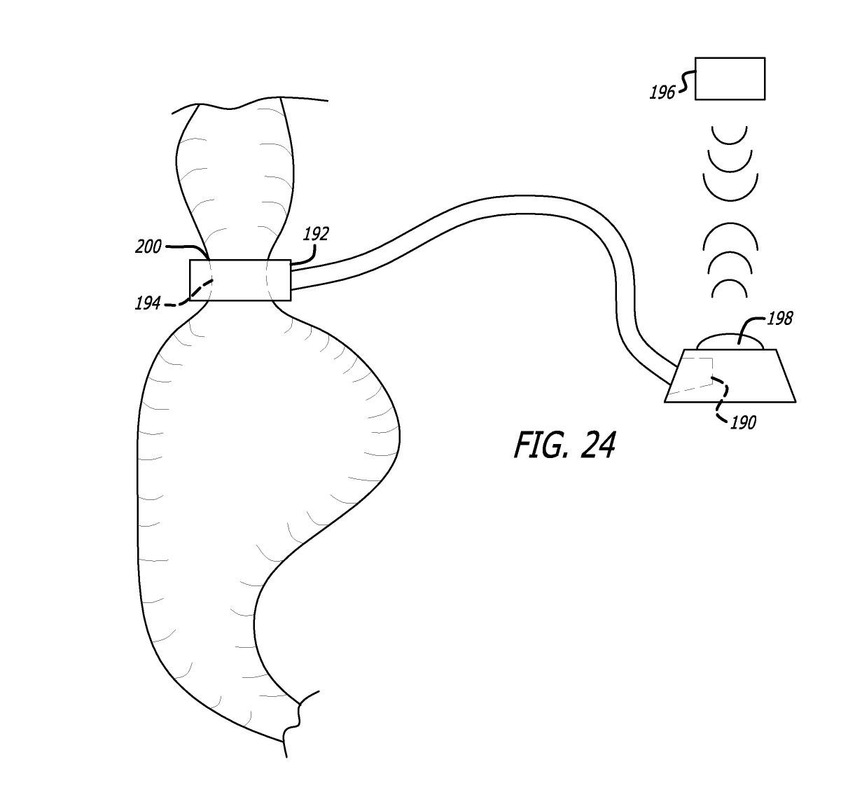

Optionally, the embodiments of the bladder assemblies disclosed herein can incorporate one or more wireless sensors to measure parameters such as pressure, flow, temperature, tissue impedance to detect tissue erosion, slippage of the gastric band, stoma diameter (via ECHO or sonomicrometry) for erosion, slippage or pouch dilatation. These sensors can be implanted in the balloon portion of the gastric band, in the bladder, in the injection port, or anywhere in the system to monitor, for example, pressure. Thus, a sensor could be implanted in the band to measure intra-band pressure or the contact pressure between the gastric band and the tissue enclosed within the band. Similarly, a sensor could be implanted in the bladder to measure fluid pressure within the system. These sensors are wireless and they communicate with an external system by acoustic waves or radio frequency signals (EndoSure® Sensor, CardioMEMS, Inc., Atlanta, Ga. and Ramon Medical Technology, a division of Boston Scientific, Natick, Mass.). In one embodiment, shown in FIG. 23, a pressure sensor 190 is implanted in the gastric band 192 which encircles stoma 194. The sensor 190 communicates a signal wirelessly (using acoustic waves for example) to external system 196 which will analyze the signal. If, as an example, the sensor indicates that the intra-band pressure or the contact pressure between the band and the stomach is low (perhaps 5 mmHg), this might be an indication that: (1) the bladder 198 has transferred all of its fluid to the balloon portion 200 of band 192 and needs to be refilled; or (2) there is a fluid leak in the system; or (3) the bladder is not working properly to continuously maintain the correct pressure at sensor 190. Alternatively, as shown in FIG. 24, sensor 190 is implanted in injection port bladder assembly 198 to measure fluid pressure. The signal from the sensor 190 is transmitted wirelessly to external system 196 to monitor the pressure in the bladder. If the bladder pressure falls too low, the bladder can be refilled as described above for FIGS. 9-13. By wireless monitoring intra-band pressures, patient management can be improved. For example, if pressures are higher or lower than desired for a given system compliance curve, then fluid can be removed or added respectively to the bladder in the system, after factoring other aspects of the patient's status. If the pressure is in the correct range for a given system, then the surgeon may chose not to adjust the band and instead counsel the patient to improve weight loss by life style improvements.

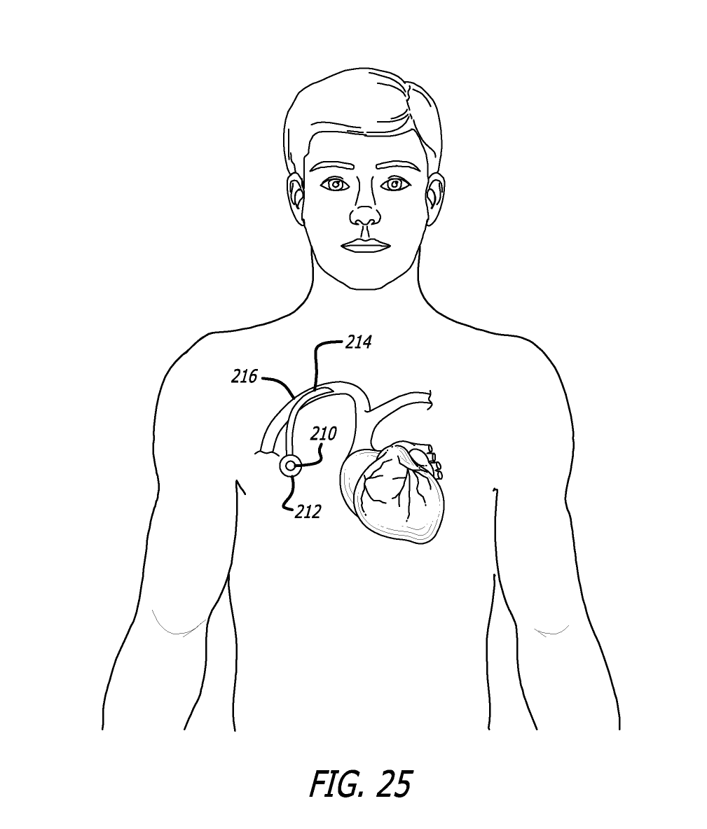

The bladder assembly disclosed herein also can be used with a venous access catheter to reduce the likelihood of clotting or hemostasis in the catheter. One of the greatest challenges with venous access catheters is their propensity to thrombose resulting in a loss of patency. These catheters are typically implanted in the subclavian vein and often include an implanted vascular access port. These vascular access ports and catheters are quite stiff having little or no fluid compliance. Central Venous Pressure is relatively low, ranging normally from 2-6 mmHg, with a pulsatile waveform. Because of the stiffness of the vascular access ports there is little distension of the inside of the access port in response to the pulsatile venous pressure waveform. Consequently, fluid within the catheter is stagnant. Hemostasis results in coagulation or clot formation. In one embodiment, as shown in FIG. 25, a compliant bladder 210 inside a port 212 may act like a trampoline and distend in response to the pressure waveform. In so doing it may cause the blood or other fluid column inside the catheter 214 to move back and forth constantly. This may prevent or delay hemostasis and clotting and result in a catheter that remains patent longer. In this embodiment, the catheter 214 is inserted in a vessel 216 (vein or artery) for infusion or withdrawal of fluids. Such systems are well known in the art (see e.g., Vital-Port® Vascular Access System, Cook Medical, Bloomington, Ind.).

With respect to any of the embodiments of the bladder disclosed herein, the bladder can be used as a drug delivery reservoir and a drug delivery pump. The bladders have an elasticity that generates a pressure on the fluid in the bladder. A drug can be injected into the bladder so that the bladder fills and expands. Due to the elasticity of the bladder, the fluid/drug is under pressure. The drug can be infused into a patient from the bladder at a controlled rate.

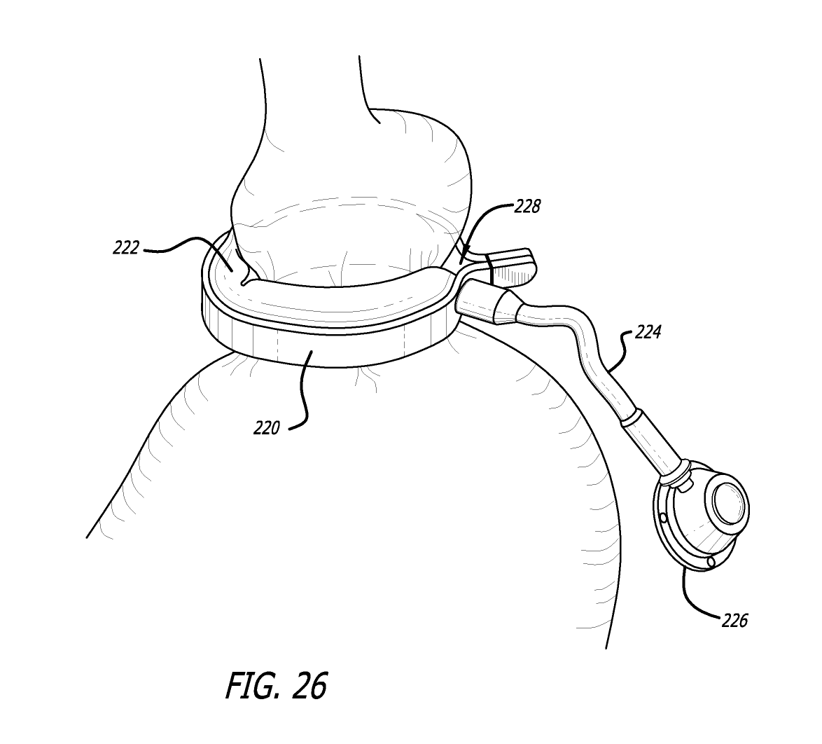

In one alternative embodiment as shown in FIG. 26, the balloon portion 222 of a gastric band 220 is formed of an elastic material so that as the balloon is filled with a fluid, it will elastically expand. In this embodiment, as the stomach tissue volume encircled by the gastric band 228 gets smaller when the patient loses weight, the balloon portion 222 will expand because fluid from the port 226 and tubing 224 will automatically flow into the balloon in order to keep a constant (predetermined) pressure on the stomach. The port 226 and the tubing 224 contain about 9 mL fluid, so the balloon has a good capacity for expansion as the stomach reduces in size. The port also can be replenished with fluid as described herein.

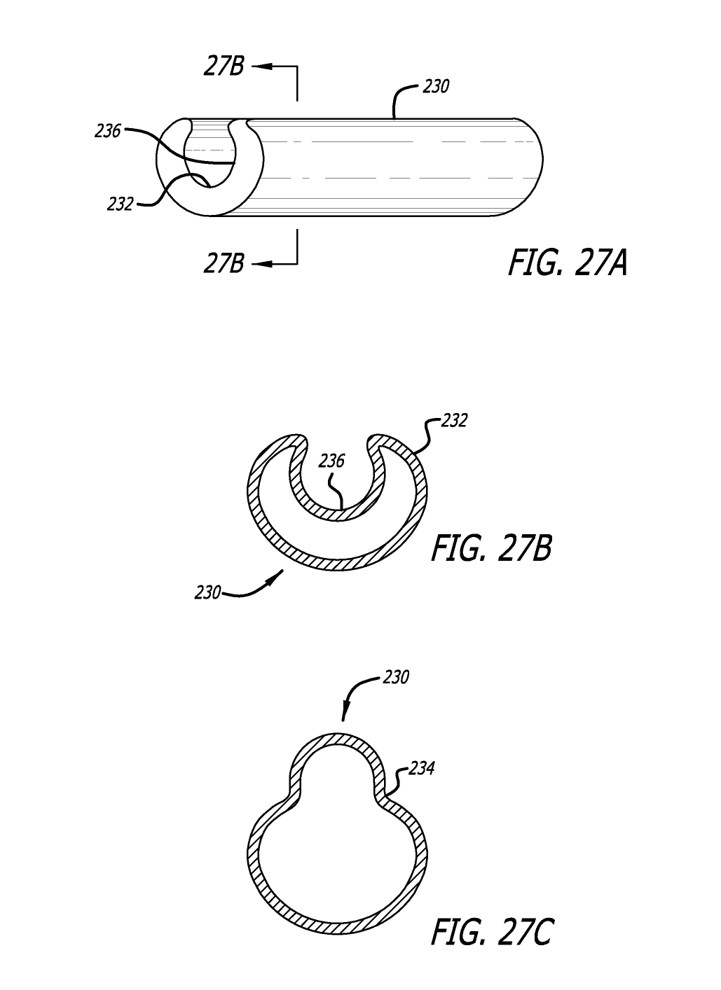

In one embodiment, bladder 230 has a unique cross-sectional shape that will achieve a desired pressure/volume curve utilizing both the material properties of the bladder (elastic material) as well as changing the cross-sectional shape. As shown in FIGS. 27A-27C, the bladder 230 has a folded configuration 232 (FIG. 27B) and an unfolded configuration 234 (FIG. 27C). In the folded configuration 232, the bladder 230 has a longitudinal fold 236 providing a very low profile for minimally invasive delivery. When fluid is then added to the bladder 230, it will pop open or unfold to the unfolded configuration 234 where the elastic properties of the bladder and its unique shape will pressurize the fluid. This embodiment can be incorporated into most of the bladder systems disclosed herein (e.g., FIGS. 2-8, 13, 13A, 15 and 23-26). In another embodiment, the bladder 230 can have more than one longitudinal fold, similar to longitudinal fold 236, spaced around the circumference of the bladder. In the folded configuration, such a bladder would have very low profile for minimally invasive delivery.

In one embodiment, multiple bladders are connected together by flexible tubing in order to maintain the pressure setting made by the physician during a routine gastric band adjustment. These bladders, connected in series, work not by holding an exact pressure, rather pressures can change with volume, thus these bladders allow the fluid volume based adjustments to still be made by the physician and thereby allow pressures to vary slightly with volume changes, but at a very slow rate as a function of volume. In other words, the slope of the compliance curve of the system, approximately 10 mmHg/mL, is relatively flat within a desired range of intra-luminal or contact pressure optimally from about 40 mmHg to about 150 mmHg, which range ideally is above the Green Zone pressure. More preferably, intra-luminal or contact pressures from about 35 mmHg to about 65 mmHg should provide optimal weight loss and keep the patient above the Green Zone. The multiple bladder configuration does not alter the settings made by the surgeon when adjusting the band, rather it maintains the pressure state to a greater extent above the Green Zone. The intra-luminal or contact pressures that are above the Green Zone are passively and continuously maintained without any outside mechanical, electrical or other feedback sensing forces and corrective adjustments, but rather are maintained hydraulically due to the specific elasticity of the bladders that are in fluid communication with the balloon portion of the gastric band and thereby provide a pressure on the fluid within the band. Importantly, with the present invention comprising multiple bladders, physicians do not have to change the way they make adjustments to the gastric band; they will, however, be making fewer adjustments over time since the bladders maintain the physician adjusted pressures that are higher than the typical Green Zone pressures for a time period longer than with just the gastric band alone. In determining the optimal intra-luminal pressures using the bladders disclosed herein, the physician should be mindful of a patient's intra-abdominal pressure of about 5 mmHg to about 9 mmHg (see DeKeulenaer, et al., Intensive Care Medicine; 2009; disclosing 9-14 mmHg), which could affect the bladder pressure and intra-luminal pressure as is discussed more fully infra.

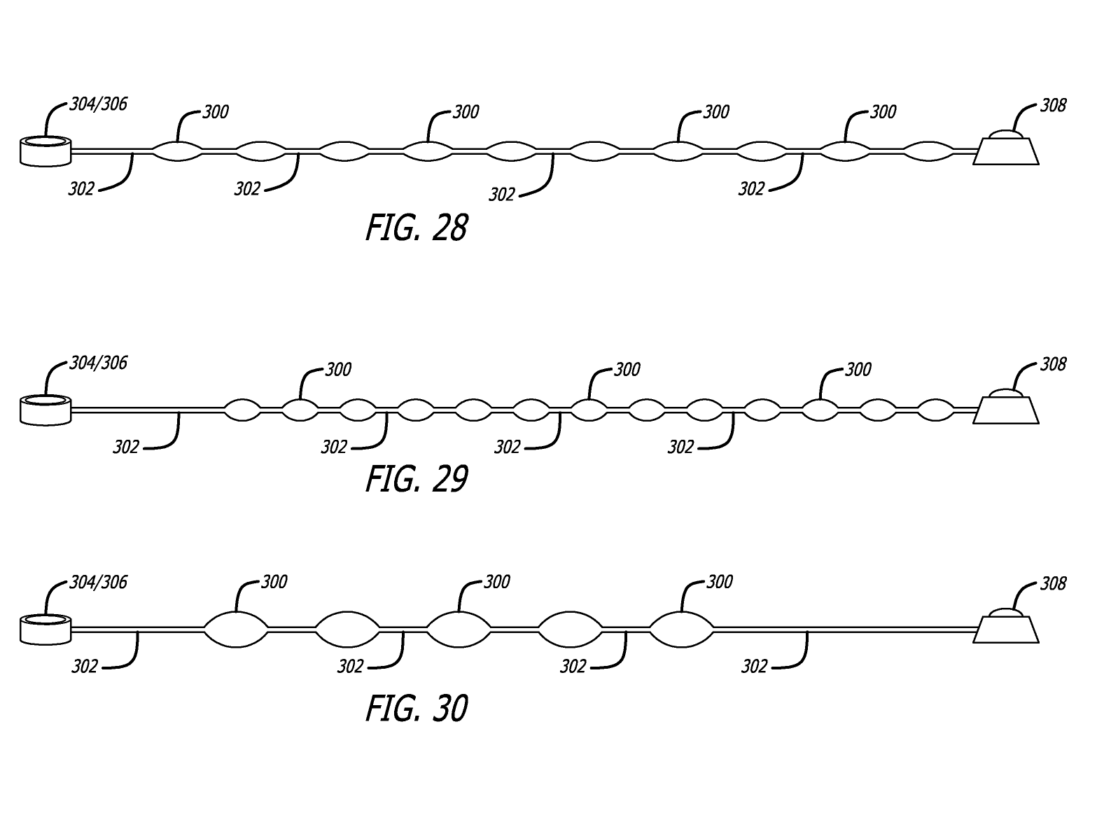

In one embodiment, as shown in FIGS. 28-31, multiple bladders 300 are connected serially by flexible tubing 302. In this embodiment, the bladders are formed from an elastic material that is expandable (and deformable) when a fluid is injected into the bladders 300. The flexible tubing 302 is formed from a material that is the same as or different from the material of the bladders 300, and is kink resistant yet highly flexible. When the bladders 300 are filled with a fluid and expand radially outwardly, they become less flexible to bending longitudinally thereby requiring that the tubing 302 connecting the bladders 300 be more flexible and kink resistant. Preferably, the flexible tubing 302 has a small diameter, is kink resistant, and will not appreciably change the pressure or compliance of the system when the tubing bends. In other words, the tubing decouples bending in the bladder assembly from changing the pressure in the bladders and even when the tubing 302 is severely bent little pressure change will occur in the bladders 300. Further, bending the tubing 302 does not alter the P-V relationship in the bladders 300. In fact, the entire bladder assembly is kink resistant, therefore severe bending does not appreciably affect the P-V relationship in the bladders. The flexible tubing 302 is connected at its distal end to the balloon portion 304 of a lap band 306 or to tubing leading to the balloon portion. At its proximal end, the flexible tubing 302 is connected to fill port 308 (or to tubing leading to the fill port), which is used to inject fluid into the system in order to expand the bladders, and thereby expand the balloon portion 304 of the lap band 306. The length of the tubing from the fill port is important. There should be sufficient length to ensure that the bladders are well within the abdominal cavity so that they do not become adhered to or compressed by/within the abdominal wall. Thus, a minimum length of tubing between the port 308 and the first bladder would be required. Also, a minimum spacing between bladders is desired so that even if the tubing 302 between adjacent bladders 300 is bent 180°, the adjacent bladders do not touch each other.

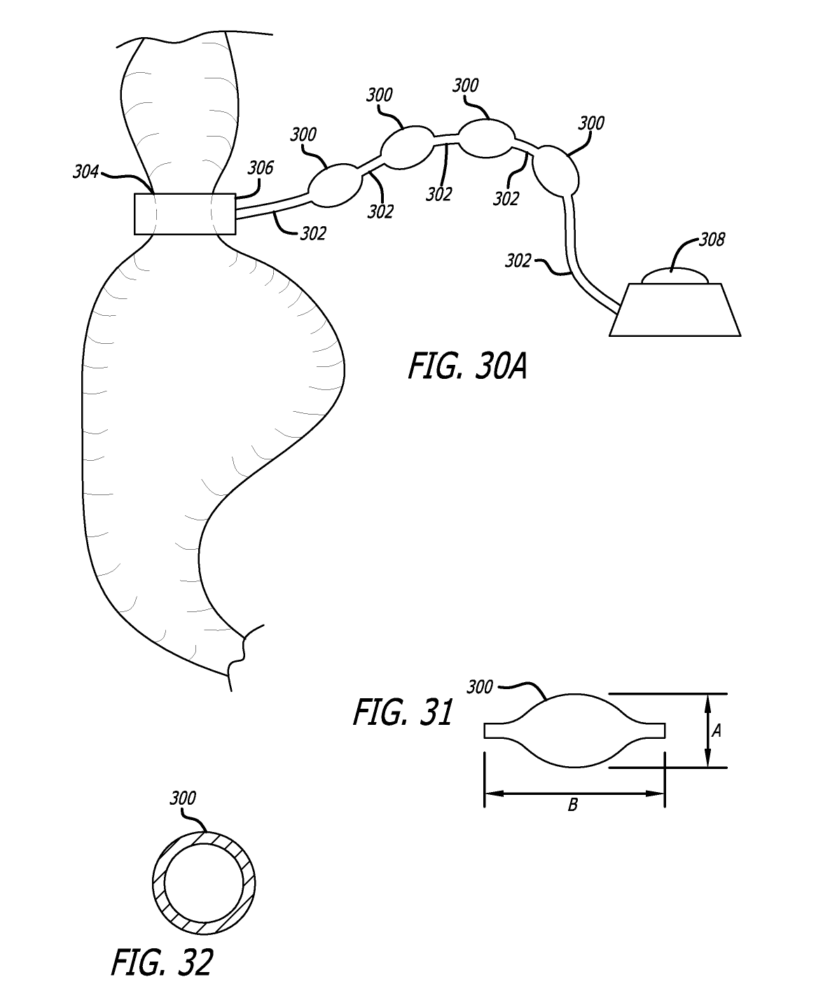

Referring to FIG. 30A, the bladder assembly preferably is positioned in the abdominal cavity (or the peritoneal cavity), as is the gastric band. The fill port 308 typically is placed just under the skin so that it may be accessed by the physician when refilling the bladders, therefore it is not in the abdominal cavity. Since the bladder assembly with bladders 300 aligned serially as shown in FIG. 30A is in the abdominal cavity, the intra-luminal and contact pressure will be unaffected by changes in atmospheric pressure. For example, a patient having a gastric band 306 might be traveling in the mountains at elevations up to 10,000 to 12,000 feet of altitude, or flying in an airplane where the cabin pressure is equivalent to 5,000 to 6,000 feet of altitude. Because both the balloon in the gastric band and the bladders 300 are exposed to abdominal pressure, and the bladders lack an outer housing, the intra-luminal and contact pressures that the bladders maintain is not affected by changes in atmospheric pressure. Therefore if atmospheric pressure should change due to a change in elevation, the intra-luminal and contact pressures do not change. In contrast, if a constant pressure pump were used to maintain intra-band pressure at a specific level, changes in atmospheric pressure will result in changes to intra-luminal and contact pressures and thereby cause the patient to experience the gastric band tightening (atmospheric pressure is lower) or loosening (atmospheric pressure is higher). Thus, as shown in FIG. 30A, the abdominal pressure (P_{abdominal}) is essentially the same on both the bladders 300 and the balloon portion 304 of the lap band 306. Any change in atmospheric pressure (P_{atmospheric}) does not impact the intra-luminal or contact pressure because both the bladders and the balloon/band are acted upon equally by the change in the atmospheric pressure. This is shown below by the following relationship where the balloon-band pressure is the left side of the equation and the bladder pressure is the right side of the equation.

Pᵢₙₜᵣₐ₋ₗᵤₘᵢₙₐₗ+P_{abdominal}+P_{intra-band}=P_{abdominal}+P_{bladder}

The P_{abdominal} is offsetting, therefore

Pᵢₙₜᵣₐ₋ₗᵤₘᵢₙₐₗ+P_{intra-band}=P_{bladder}

and

Pᵢₙₜᵣₐ₋ₗᵤₘᵢₙₐₗ=P_{bladder}−P_{intra-band}

There is anecdotal evidence that patients with lap bands have reported an uncomfortable tightening of their bands when they have flown in an airplane. The present invention bladder assembly, such as that shown in FIG. 30A, eliminates a change in intra-luminal and contact pressure due to changes in atmospheric pressure as disclosed. In other words, the intra-luminal and contact pressure generated by the bladders does not vary with changes in atmospheric pressure.

Depending upon the type of gastric band used, it may be necessary to vary not only the diameter and the length of the bladders 300 but also the number of bladders used, the material used in the bladders, and the P-V relationship of the bladders. In this regard, as shown in FIG. 31, the diameters of the bladders 300 shown in FIGS. 28, 29 and 30 are respectively 8 mm (0.31 inch), 9 mm (0.35 inch), and 15 mm (0.59 inch). Further, the lengths of the straight segment of the bladders shown in FIGS. 28, 29 and 30 are respectively 32.0 mm (1.26 inch), 24.3 mm (0.96 inch), and 36.6 mm (1.44 inch). The outer diameter of the unexpanded bladders is preferably less than 15 mm (0.59 inch) which corresponds to the inner diameter of a trocar used in delivery of the gastric band and bladders. The length of the straight segment of the bladders 300 can vary from 10 mm (0.39 inch) to 50 mm (1.97 inch), however, the longer the segment more difficult it will be for the bladders to negotiate bends during delivery and the greater the tendency to kink. It is desired to keep the overall length of the bladders 300 and connective tubing 302 from 45 cm to 60 cm (17.72 inch to 23.62 inch). The wall thickness of bladders 300 can range from 0.25 mm (0.0098 inch) to 1.0 mm (0.039 inch), and a preferred wall thickness is 0.62 mm (0.024 inch). While these dimensions for the bladders 300 are precisely disclosed, it is clear that other dimensions for the bladders 300 may be appropriate given different conditions, including different types of lap bands, patient physiology, or other similar factors. Referring to FIG. 32, the typical cross-section for bladders 300 is circular, or substantially circular. As will be seen, other cross-sectional configurations may be more appropriate in order to increase or decrease the pressure provided by the bladders within the system. The dimensions of the bladder and tubing disclosed herein are representative and can vary depending on factors such as the type of LABG used with the bladder and the size of the patient (i.e., a very short patient compared to a very tall patient will require different sized bladders and length of tubing).

For any of the bladders disclosed herein, the bladders can be connected to the tubing leading to the balloon portion of a gastric band at one end, and to the tubing leading to a refill port at the other end. Referring to FIG. 30A, a bladder assembly 302 such as that shown in FIG. 30, is connected by tubing 302 at its distal end to the balloon portion 304 of the gastric band 306 and at its proximal end to a port 308 used to refill the system with fluid.

It is desirable for the in-line bladders to have a certain P-V compliance characteristic over a certain pressure range, such as 50 mmHg to 200 mmHg for the AP BAND. It takes considerable fluid volume in the bladders, however, just to get to the working pressure range if the P-V compliance is maintained. For example, if the desirable P-V compliance is 10 mmHg/mL over the working pressure range (50-200 mmHg), then it takes 5 mL of fluid volume (50 mmHg over 10 mmHg/mL=5 mL) just to bring the in-line bladders to the working range. Thus, it may be necessary to pre-stress the bladders in order to minimize the total volume of fluid thereby both minimizing the size of the bladders and reducing the amount of fluid volume required to achieve a certain P-V compliance over the specified pressure range. If the bladders are smaller because they are pre-stressed, they will be less invasive in the body and easier to implant through a trocar having a 15 mm (0.59 inch) inner diameter through which a gastric band is typically inserted.

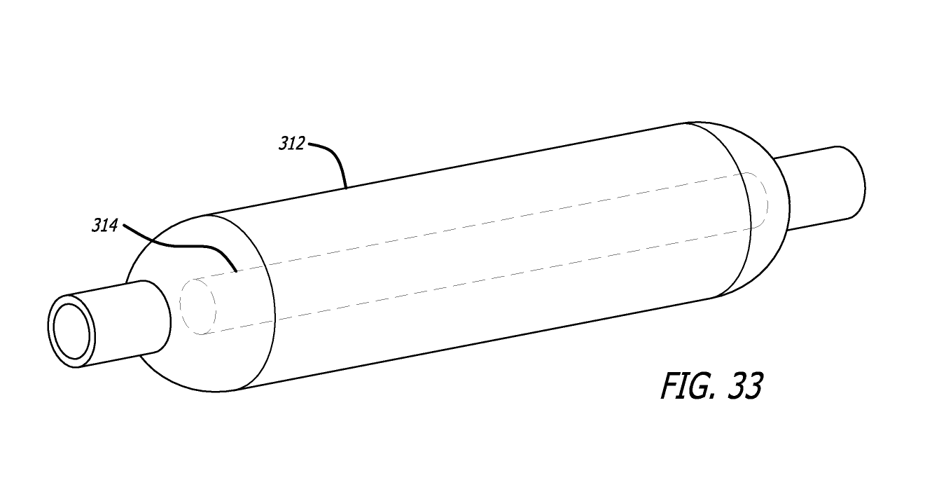

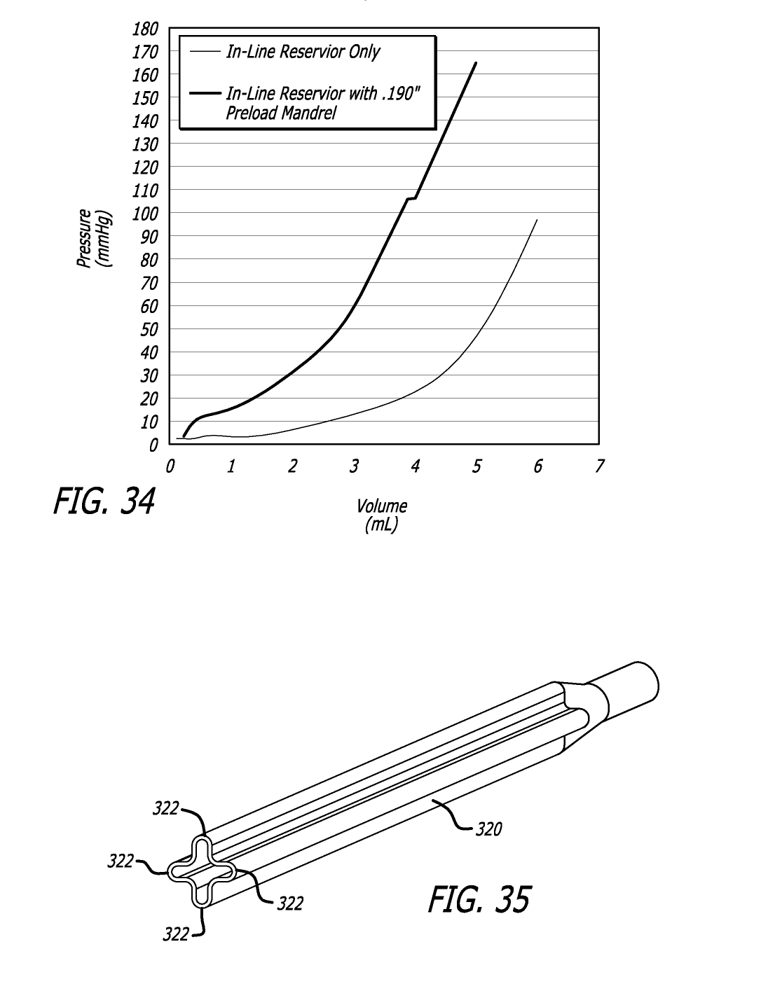

One way to pre-stress the bladders is to insert a space occupier or mandrel into the bladder. As shown in FIGS. 33 and 34, bladder 312 is similar in configuration to bladders 300 shown in FIGS. 28-31. In this embodiment, a mandrel 314 is inserted inside bladder 312. In one experiment, the mandrel had an outside diameter of 4.8 mm (0.19 inch) and was of sufficient length to extend along a substantial portion of the length of the bladder 312. As can be seen in the chart in FIG. 34, the bladder without a mandrel (or space occupier) required 2.5 mL of fluid to generate approximately 10 mmHg of intra-band pressure while bladder 312 with the mandrel 314 inserted required less than 0.5 mL of fluid to reach 10 mmHg of intra-band pressure.

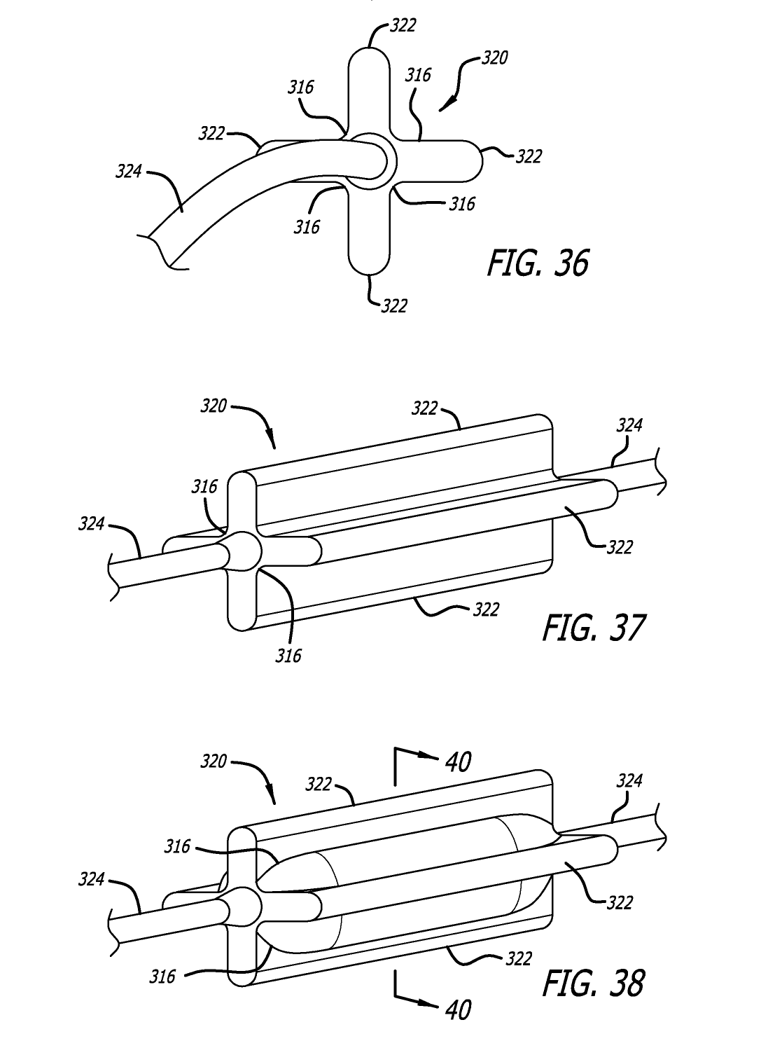

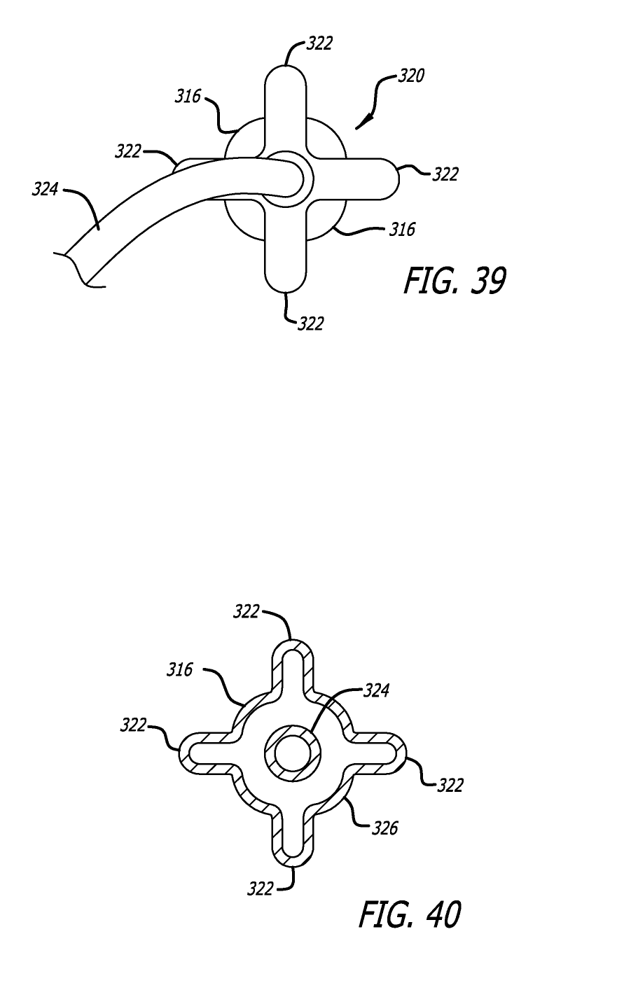

As disclosed, the bladders need not have a circular cross-section such as that shown in FIG. 32. For example, as shown in FIGS. 35-40, bladders 320 have a cross-section in which three or more wings 322 extend radially outwardly. In this embodiment, there are four wings 322 (a cross-shape), however, this number can vary from two to five wings or more depending upon the particular application. Like the bladders 300 disclosed in FIGS. 28-32, bladders 320 are aligned serially and are in fluid communication with each other with a flexible tubing 324 positioned between the bladders. One reason to provide bladders with wings, or other non-circular cross-sections, is so that the bladders can be pre-stressed. Thus, a pre-stressed cross-shaped bladder can provide higher fluid pressure for a given volume than a bladder with a non-pre-stressed circular shape. A circular shaped bladder can also be pre-stressed by stretching an elastic tube with an ID smaller than the OD of the mandrel inside of it. The wing design provides energy storage by bending rather than pure stretch/tension that would occur in a circular design. In other words, the L-shaped portion (inward most curves) on the winged bladder will bend outwardly (as opposed to merely stretching like a circular bladder) when filled with fluid, thereby creating pressure on the fluid because these L-shaped portions want to return inwardly to their original configuration. This allows an increase in the wall thickness of the silicone and still stay within desired compliance ranges. To achieve the compliance range with a circular design would require very thin walls which could be more difficult to manufacture consistently and could be less durable and would also permit a higher saline leakage rate.

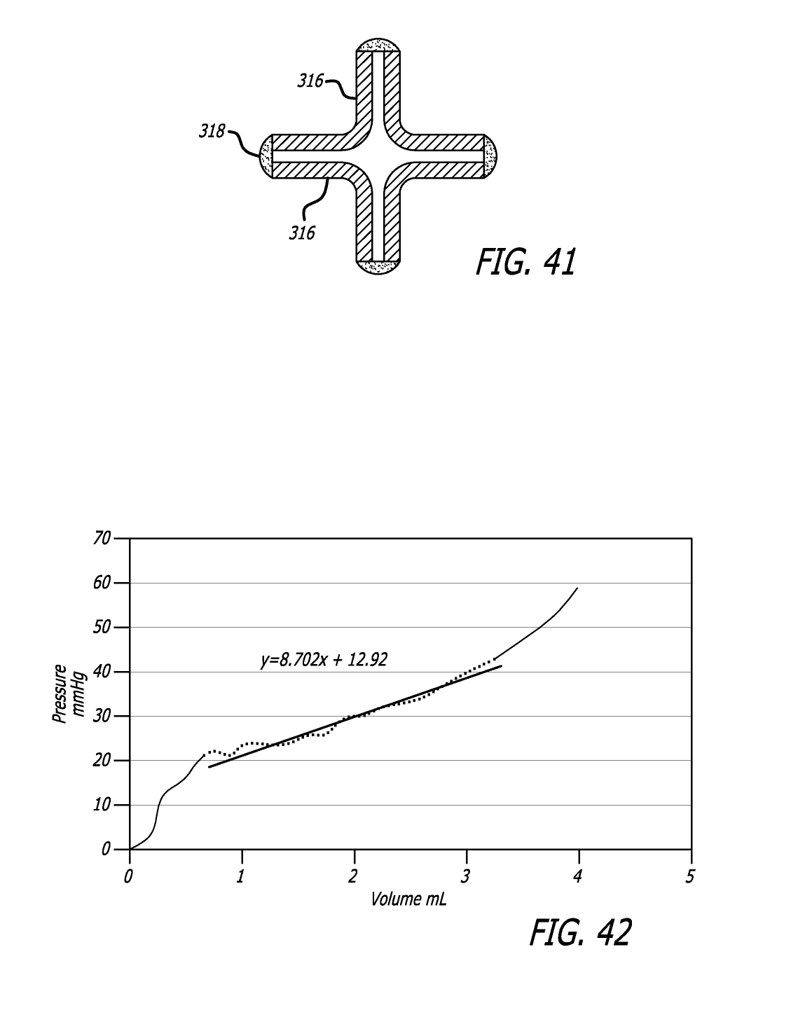

The bladders shown in FIGS. 35-40 can have four wings and be cross-shaped as shown, have three wings and be Y-shaped (not shown), or have five wings and be penta-shaped (not shown). The diameter prior to expansion can range from about 3 mm (0.12 inch) up to about 25 mm (0.98 inch), while the length can range from about 15 mm (0.59 inch) up to about 5.0 cm (1.97 inch). In one embodiment, the bladders 320 are formed from a silicone or silicone rubber material that is U-shaped and then opened to form a pre-stressed L-shaped portion 316 as shown in FIG. 41. In this embodiment, four of the pre-stressed L-shaped portions 316 are connected by silicone adhesive caps 318 as shown in FIG. 41. The bladders 320 having this configuration are in a pre-stressed condition so that as fluid is injected into the bladders the L-shaped portions 316 will evert radially outwardly (bending outwardly) and it will require a substantially higher pressure to evert the pre-stressed L-shaped portions by overcoming the elastic nature of the silicone or silicone rubber pushing radially outwardly. The wall thickness of any of the bladders disclosed herein can range from 0.03 mm (0.012 inch) to 1.57 mm (0.062 inch), but these dimensions can be either thinner or thicker depending upon a particular application. One preferred thickness for the bladder wall is 0.89 mm (0.035 inch). A relatively thicker wall equates to higher durability and less leakage, and it may be more resistant to bending and stretching.

An experiment was conducted on a bladder 320 as shown in FIG. 41, in which the diameter from wing tip to wing tip 322 was approximately 12.5 mm (0.49 inch) while the length of the bladder 320 was 44 mm (1.7 inch). The bladder 320 was connected to a Realize Band® and pressure measurements were taken at various fill volumes. As shown in FIG. 42, the pressure-volume compliance curve meets the desired specification for the Realize Band®. Due to pre-loading of the bladder 320, it took just 0.7 mL of fluid to bring the intra-band pressure in the balloon portion of the Realize Band® to just above 20 mmHg (at an average rate of about 29 mmHg/mL). For the next 3 mL of additional volume, the intra-band pressure went from 20 mmHg to 45 mmHg (at an average rate of about 9 mmHg per mL). A compliance of less than 10 mmHg/mL is desired in order to maintain the desired pressure in the Green Zone over a significantly larger range of intra-band volume. Importantly, for this type of gastric band, the bladder 320 was able to maintain operating pressures corresponding to the Green Zone, which in this embodiment was about 20 mmHg to about 40 mmHg, by adding just 3.0 mL of fluid to the bladder 320. By adding pre-stressed bladders 320 in series, the band would operate in the Green Zone with even less fluid added to the bladders (less than 0.7 mL) to reach the low end of the Green Zone. With the pre-stressed bladder, intra-band, contact and intra-luminal pressures that are much higher than Green Zone pressures can be more easily achieved for a given fluid volume. The use of pre-stressed bladders with the band results in the slope of the P-V compliance curve of the overall system to be flatter than the slope of the P-V compliance curve of the gastric band alone.

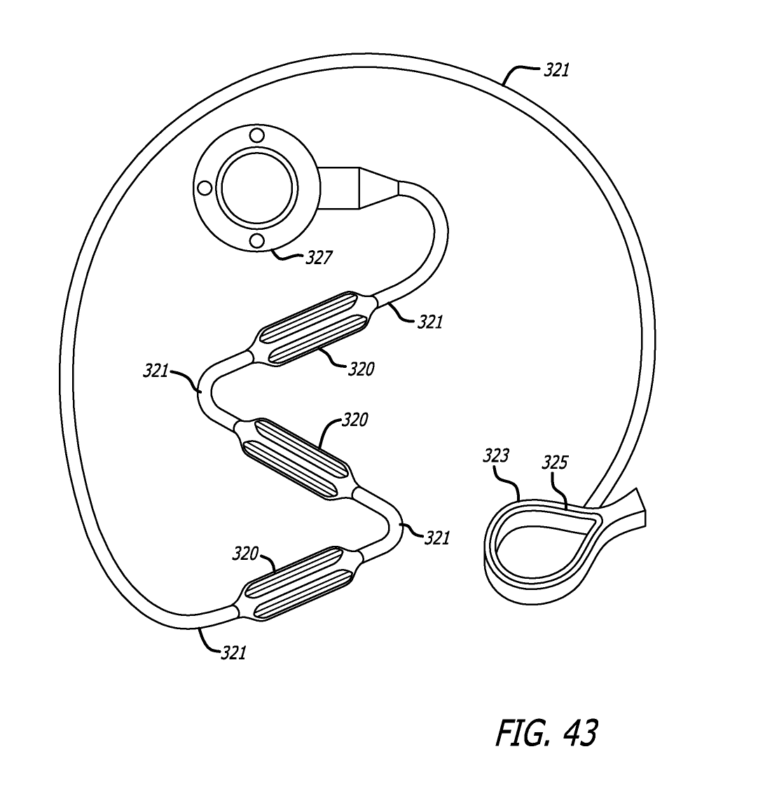

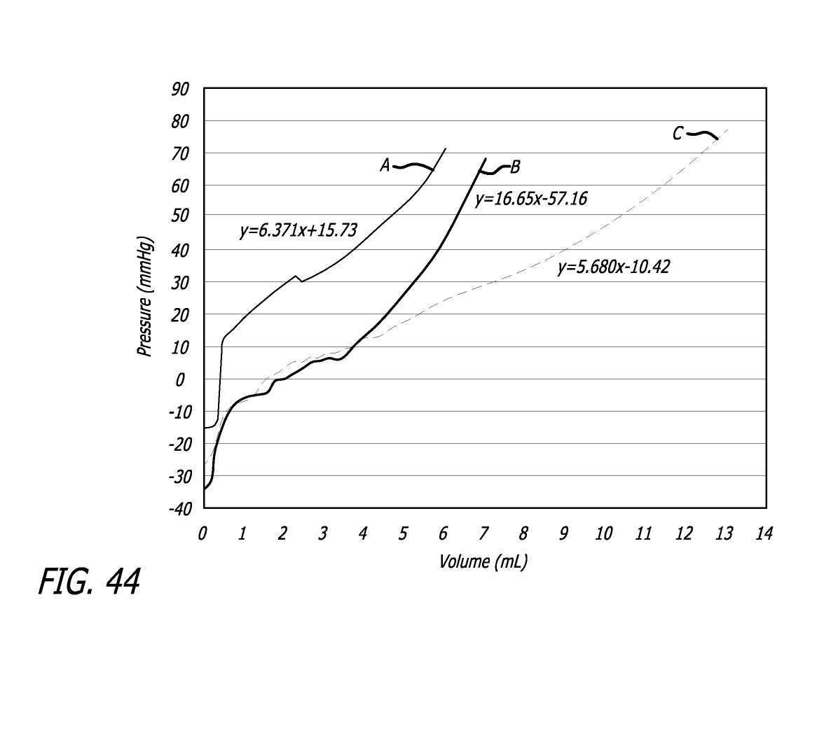

In another experiment, as shown in FIGS. 43 and 44, three bladders 320 are connected serially by kink resistant flexible tubing 321. In this embodiment, the bladders have five wings as previously described and are pre-stressed. The bladders 320 are connected to the balloon portion 325 of a gastric band, in this case a Realize Band® 323. At the other end, the bladder assembly is attached to refill port 327. Fluid was injected through the refill port 327 and into the bladders 320 and the results are recorded in the pressure vs. volume curves shown in FIG. 44. Referring to FIG. 44, curve A is the pressure-volume compliance curve of the in-line bladders only. Curve A shows the initial quick jump in pressure with very little fluid volume change added to the bladders 320. This is due to the pre-load feature of the bladders 320 as previously described. The pressure-volume compliance of the in-line bladders 320 is about 6.4 mmHg/mL between the pressures of 25-40 mmHg. Curve B is the pressure-volume compliance curve of the Realize Band® only. This experiment was conducted with the band encircling a 24 mm diameter teflon mandrel to simulate encircled stomach tissue. The pressure-volume compliance of the Realize Band® is about 16.7 mmHg/mL of fluid between the pressures of 25-40 mmHg. Curve C is the pressure-volume compliance curve of the combined system of the bladders 320 connected to the Realize Band® 323. Initially, pressure-volume compliance curve C tracks that of the Realize Band® only, however, once the pressure exceeded the initial pre-load pressure of the bladders (around 15 mmHg in this case), the pressure-volume compliance of the system reflects the characteristics of the two combined sub-components, i.e., the bladders 320 and the balloon 325. The pressure-volume compliance of the system is about 5.7 mmHg/mL between the pressures of 25-40 mmHg.

Another way to calculate the combined system pressure-volume compliance based on the pressure-volume compliance of the bladders 320 and the balloon 325 is as follows:

$$\frac{1}{p - v system} = \frac{1}{p - v band} + \frac{1}{p - v bladder}$$

$$p - v system = \frac{1}{( \frac{1}{16.7} + \frac{1}{6.4} )} = 4.6 mmHg \text{/} mL$$

The experimental value of the pressure-volume system is 5.7 mmHg/mL while the theoretical pressure-volume system is 4.6 mmHg/mL. The difference could be due to slight variations in testing and/or the linear approximation of the pressure-volume compliance of the sub-components. As the equation indicates, adding a bladder system to the gastric band would lower the pressure-volume compliance of the band regardless of whether the pressure-volume compliance of the bladder system is higher or lower than the pressure-volume compliance of the band.

Other cross-sectional shapes are contemplated such as paddle-shaped, elliptical-shaped, star-shaped and oval-shaped. These additional shapes also can be pre-stressed as desired.

In one embodiment, the bladder shown in FIG. 35 includes flexible tubing extending through the bladder. For example, as shown in FIG. 40, a cross-sectional view of a bladder 320 discloses wings 322 extending radially outwardly and flexible tubing 324 extending through the center of the bladder 320. In this embodiment, fluid has filled the bladder so that the inflated bladder 326 and the wings 322 have partially opened or spread apart due to the elastic nature of the bladder 320. The flexible tubing 324 preferably is highly flexible and can be formed from silicone rubber having an inner diameter of 3.2 mm (0.125 inch) and an outer diameter of 15.9 mm (0.625 inch). The silicone rubber tubing 324 acts as a support for the bladder 320 during bending, allowing the bladder to take a much tighter bend or curve without kinking. Further, the tubing 324 inside the bladder pre-stresses the bladder wall by occupying the central lumen of the bladder which has the same effect of inserting a mandrel in the middle of a bladders as previously described.

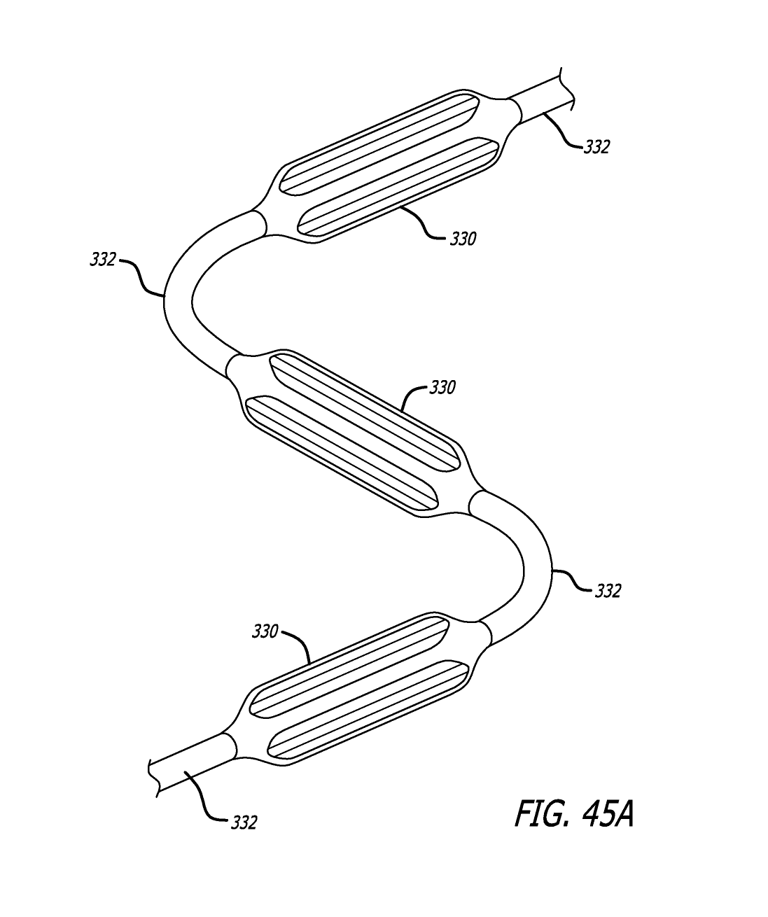

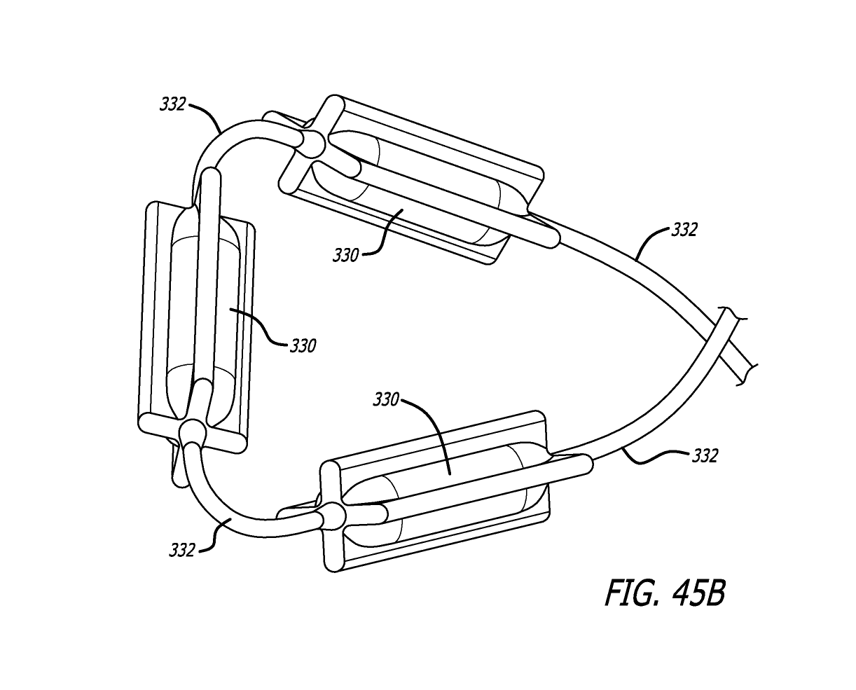

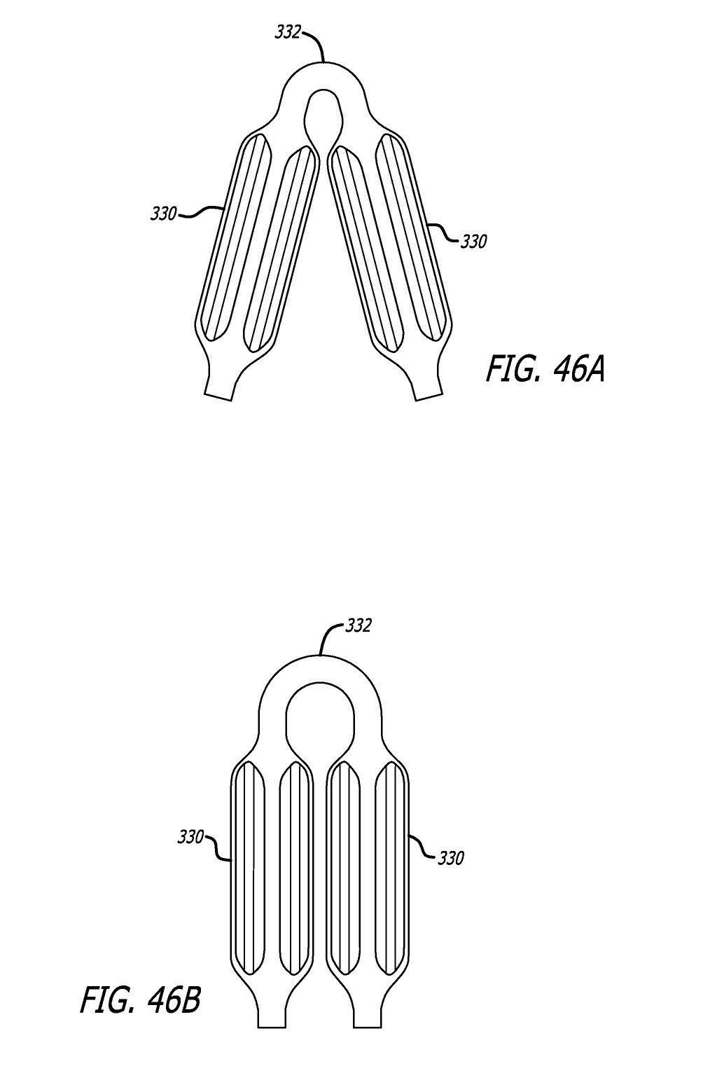

With respect to any of the foregoing bladder configurations, the flexible tubing connecting the bladders can have different configurations. For example, as shown in FIGS. 45A and 45B, the bladders 330, which are similar to those previously described, are connected by flexible tubing 332 that is formed of a silicone rubber material that is not only highly flexible but also kink resistant. In this embodiment, it can be seen that the flexible tubing 332 extends through the bladders 330, however, this is not necessary in order for the system to operate. The minimum length of flexible tubing 332 between bladders 330 should be long enough to allow a 180° bend in the tubing 332 without adjacent bladders hitting each other. Thus, in FIG. 46A, the length of tubing 332 is too short because the bladders 330 are touching and this may impede delivery of the bladders during the implant procedure. In FIG. 46B, the length of the tubing 332 is sufficient to allow a 180° bend in the tubing so that the adjacent bladders do not interfere with each other. In order to make the 180° bend shown in FIG. 46B, the minimum length of tubing 332 between bladders is one-half of the circumference of a circle that has the same diameter as that of the bladder 330. The tubing can be attached to each end of the bladders by conventional means such as use of adhesives or similar fastening materials known in the art to form a fluid tight seal between the tubing and the bladders.

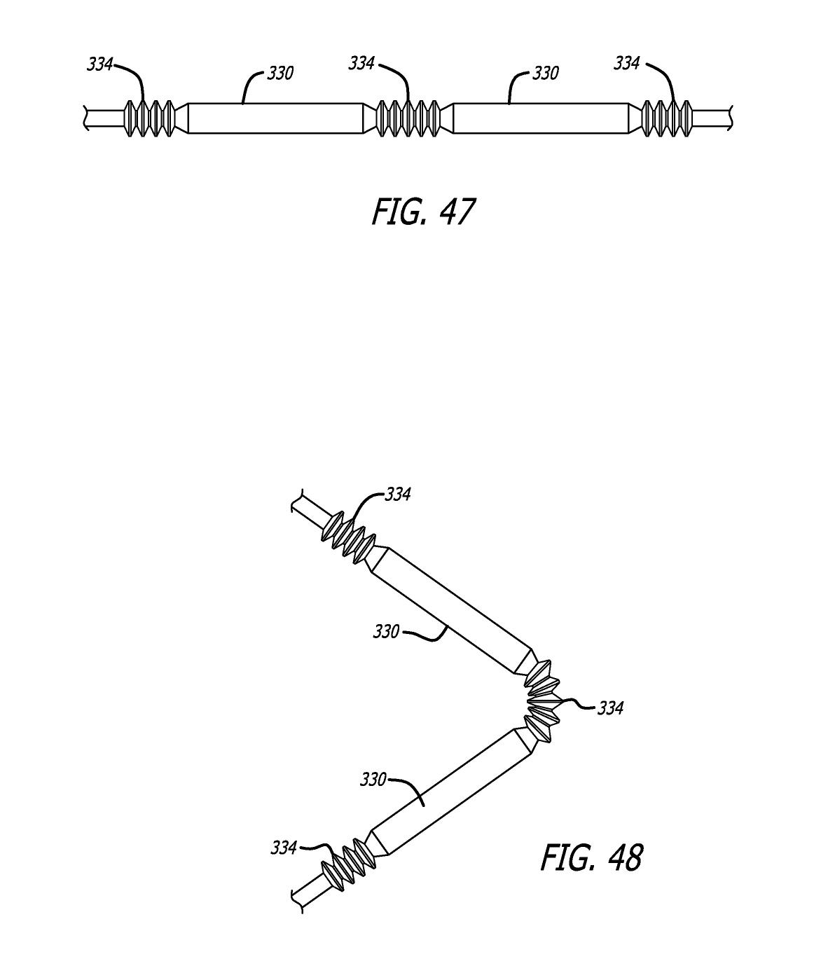

In another embodiment, as shown in FIGS. 47-48, the bladders 330 are connected by bellows-shaped tubing 334 (or corrugated-shaped). As can be seen, in this embodiment the bellows-shaped tubing allows the assembly to take very sharp bends without kinking or restricting fluid flow from one bladder to the next. Importantly, the entire bladder assembly is kink resistant and any bending in the entire assembly does not affect the pressure in the bladders.

Importantly, the flexible tubing as disclosed herein is not only flexible and kink resistant, but it also does not appreciably affect the pressure in the bladders when the tubing is bent. Thus, the small diameter tubing does not expand and will not change pressure or compliance in the system when bent, thereby decoupling the bending in the tubing from the system pressure.

In use, the bladders of the present invention can be incorporated in to existing gastric band systems that are already implanted in patients, or manufactured in line with gastric bands that have yet to be implanted. For example, as shown in FIGS. 28-30 and 30A, the modular design of the bladders allow for the bladders to be connected to the tubing extending from the gastric band at one end, and the refill port at the other end. Thus, referring to FIG. 30A, the bladders 30 are connected via tubing 302 to the gastric band 306 at a distal end, and to the refill port 308 via tubing 302 at the proximal end. The bladders 300 and tubing 302 are sized to be inserted through a trocar having an inside diameter of approximately 15 mm (0.59 inch) and can be attached via known connectors to the tubing already in place when the gastric band has already been implanted in a patient. Similarly, for those gastric bands that are not yet inserted in a patient, the bladders 300 and tubing 302 are built into the gastric band and refill port by the connective tubing as shown in FIGS. 28-30. It is also contemplated that the bladder assembly has metallic components that are MRI compatible and radiopaque.

In one embodiment, radiopaque markers are attached to the tubing or bladders to indicate either volume or pressure related to filling the bladders. For example, as shown in FIGS. 50-55, radiopaque markers on a bladder 300 are spaced apart and the distance between the markers can be measured both before the injecting of fluid and after injecting fluid via fluoroscopy, X-ray or any other means of imaging (ultrasound, ECHO, sonography, etc.). As the bladder expands during filling, the distance between radiopaque markers increases As the volume inside the bladders continues to increase, the distance between the radiopaque markers 301 also continues to increase. There is a direct correlation between the fluid volume inside the bladder, the spacing between the radiopaque markers, and the intra-band pressure of the entire system. For example, by measuring the distance between the radiopaque markers as fluid is injected into the bladder, this correlates to a specific volume inside the bladder, and based on the pressure-volume compliance curve of the system, will translate to the intra-band pressure.

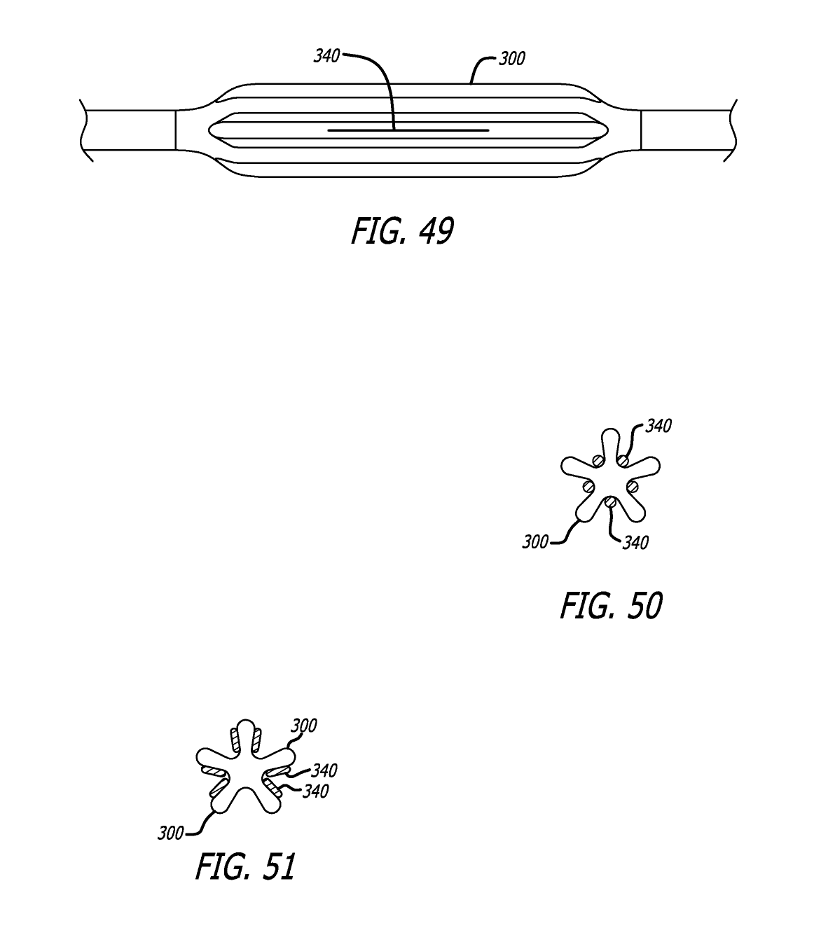

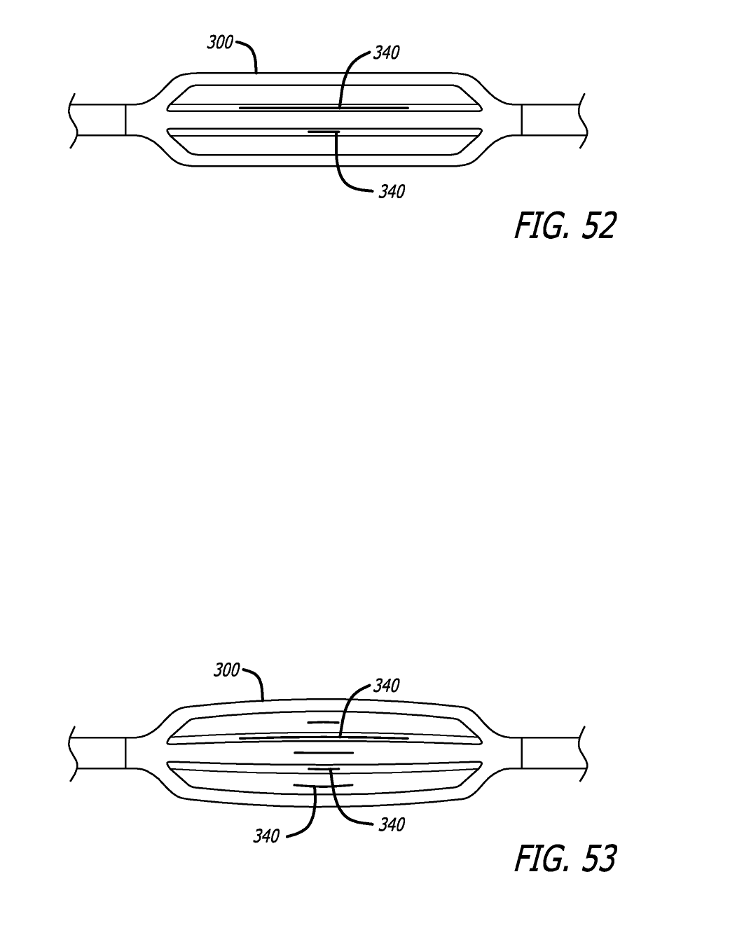

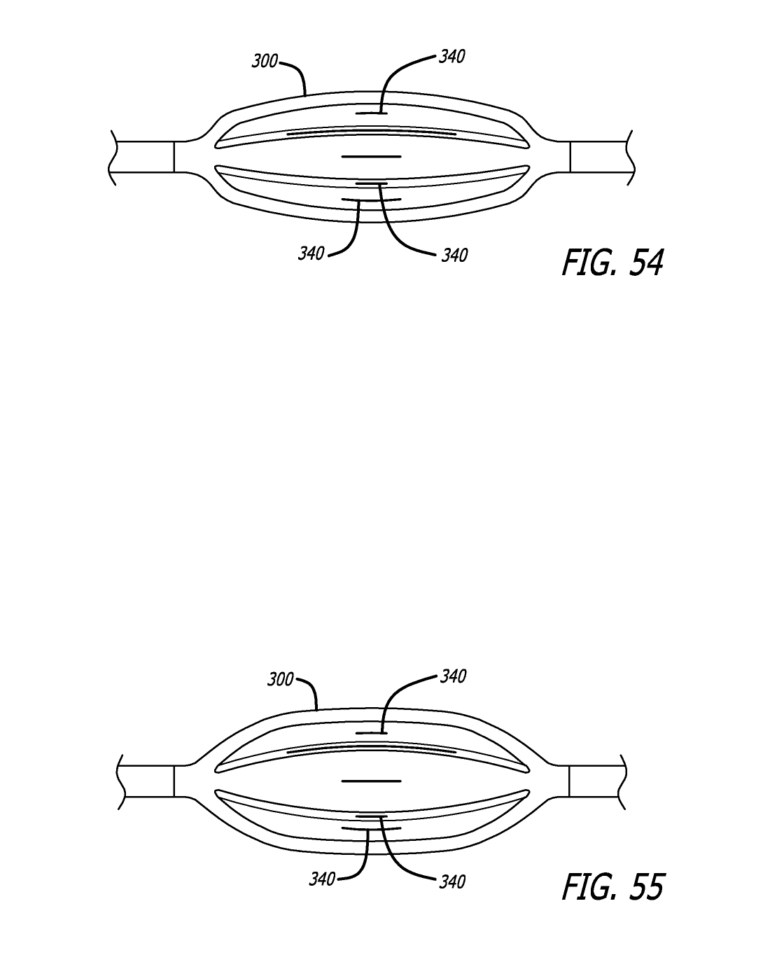

Referring to FIG. 49, a portion of a bladder assembly is shown in which bladder 300 has a radiopaque marker 340 in the form of a highly radiopaque wire imbedded in the polymer of the bladder or attached thereto by adhesives. As shown in FIG. 50, the radiopaque wires are in the valley portions of the winged bladder and are either attached by adhesives or formed into the polymer material. In this embodiment, the radiopaque wires 340 can be of the same length, or be of different lengths so that under imaging technology such as fluoroscopy, the different length wires can be easily identified, therefore determining which side of the bladder the wire is positioned relative to wires on the opposite side of the bladder. FIG. 51 shows another embodiment of radiopaque wires 340 adhered to the outer surface of the bladder or molded into the polymer material. The wires 340 in FIG. 51 are in a pattern (e.g., two side by side, one on each side of a wing, etc.) so that they can be identified under fluoroscopic imaging. FIGS. 52-55 represent a bladder 300 at various stages of fluid filling. In FIG. 52, no fluid is in bladder 300, therefore the radiopaque markers 340 have an even spacing. In FIG. 53, 1 mL of fluid has been injected into bladder 300, and the distance between the radiopaque markers is seen to have increased. Since the radiopaque markers have different lengths the spacing between adjacent wires, or between wires on opposite sides of the bladder, is easily determined. In FIG. 54, 2 mL of fluid has been injected into bladder 300 thereby increasing the distance between the radiopaque markers. Again, the different lengths of the radiopaque marker wires will assist in determining the diameter of the bladder, and hence the amount of fluid volume in the bladder which can then be used to calculate the intra-band pressure based on the known pressure-volume compliance curve of the system. Finally, with reference to FIG. 55, 3 mL of fluid has been injected into the bladder with a corresponding increase in the distance between the radiopaque markers. The distance between the radiopaque markers 340 indicates the diameter formed by the valleys of the folds as can be seen in FIGS. 50 and 51. The distance between the radiopaque markers is determinative of the diameter of the bladder, and can be calculated even when viewing the bladder under different angles under fluoroscopy, x-ray or the like. Thus, there is a good correlation between the maximum distance between radiopaque markers, thereby indicating the diameter of the bladder to the volume inside the bladder regardless of the angle at which the images were taken. This information is clinically important since the pressure-volume relationship of the bladder is known, and knowing the volume inside the bladder one can calculate the pressure inside the bladder and the intra-band pressure of the system based on the pressure-volume compliance curve of the entire system. This is a great benefit to the physician when refilling the bladders to be able to non-invasively determine how much volume has been added to system and the corresponding intra-band pressure, all based on the measurement of the spacing between the radiopaque markers. Further, as an added benefit, the radiopaque markers can be used during delivery when a gastric band is first implanted in a patient, and then later to determine the location of the various bladders in the bladder assembly. Some representative lengths for the radiopaque marker wires range from about 4 mm (0.16 inch) up to approximately 20 mm (0.79 inch). As stated, in order to assist in visualizing the radiopaque markers, the different lengths on opposite sides of the bladder will help determine the spacing between the wires, as opposed to having all wires of the same length and not being able to distinguish if two wires are side by side or opposite each other on a bladder.

Alternatively, the diameter of the bladders 300 can be determined by loading barium sulfate (BaSO4) in about 6% to 30% by weight into the polymer material (e.g., silicone) of the bladders. The bladders will be visible under fluoroscopy and the amount of fluid in the bladders can be determined by measuring the diameter of the bladders, which can then be used to calculate intra-band pressure. Similarly, the barium sulfate can be loaded into the polymer bladders at select locations such as the valley portions of the winged bladders much the same as the radiopaque wires 340 (FIGS. 49-55) with the same effect.

Importantly, the bladder assembly is modular so that a surgeon can determine at the time of surgery what size bladder assembly to use. For example, FIGS. 28-31 show different sized bladders that may be useful for a particular application. These bladder sizes can be incorporated into any type of gastric band assembly including those already on the market such as the Realize Band® (made by Ethicon Endo-Surgery, Inc.) and the Lap-Band AP (made by Allergan Inc.). Thus, prior to surgery, the surgeon simply selects the gastric band for the patient and then determines what size bladder assembly to connect to the gastric band and refill port using standard connectors that are known in the art to connect the bladder assembly in series similar to that shown in FIGS. 28-30.

The bladders disclosed herein can be formed by numerous manufacturing methods such as disclosed in co-pending U.S. Ser. No. 12/940,673, which is incorporated herein by reference thereto.

It is possible that fibrotic tissue may attach to the bladders or tubing and this could potentially impact the pressure-volume relationship in the system. To reduce the likelihood of fibrosis on the bladders, a steroid or therapeutic agent such as dexamethasone is coated onto or released from the bladders to resist development of fibrotic tissue. Further, it is contemplated that it may be desirable to coat the bladders and/or tubing disclosed herein with a therapeutic agent much the same as intravascular stents are coated. Therefore, the drug coatings disclosed in U.S. Pat. No. 7,645,476 are incorporated herein by reference.

It is to be understood that the parameters described along with the dimensions of the various bladder assemblies can vary according to a particular application. For example, the Realize Band® may have different operating pressures than the AP Band, and therefore the bladders may have different dimensions in order to maintain the pressure in the bands at a level higher than in the Green Zone for a time longer than a system without the bladders.

### Compliance and High Intra-Luminal Pressure Use

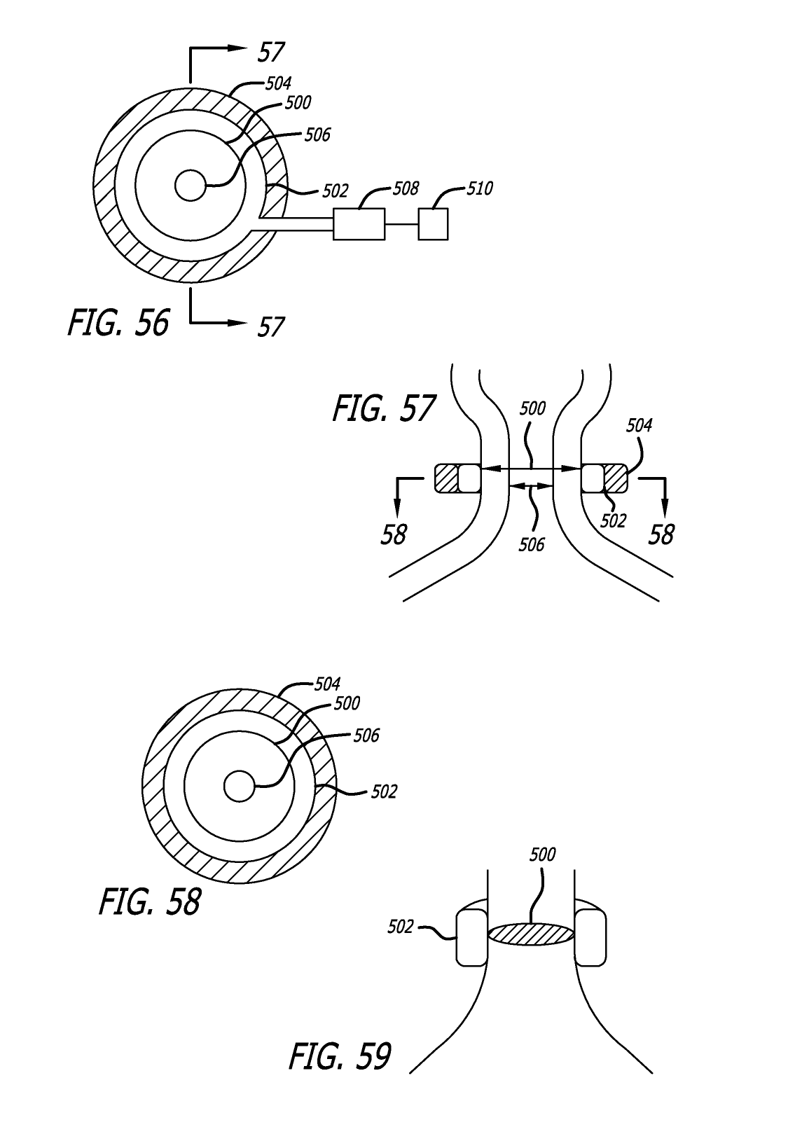

In further keeping with the invention, as shown in FIGS. 56-59, the bladders as disclosed herein, when used in conjunction with the gastric band assembly, will minimize the effects of fluid changes in the balloon portion of the gastric band and will keep the patient at intra-luminal and contact pressures higher than those in the so-called Green Zone even when there are changes to the fluid level in the assembly (e.g., fill adjustments). The one or more bladders as previously disclosed will minimize changes to the band contact area and hence the stoma area as shown in FIGS. 56-59. More specifically, the band contact area 500, which is the area of stomach tissue encircled by the balloon portion 502 of the gastric band 504, will increase or decrease in area in response to fluid level changes in the balloon portion of the gastric band. Likewise, the stoma area 506, which is the intra-luminal opening inside that portion of the stomach tissue encircled by the balloon portion of the gastric band, will also change inside in response to fluid level changes in the balloon. As can be seen in FIGS. 56-59, the band contact area 500 includes the stoma area 506. In order to minimize the changes in band contact area and stoma area in response to fluid level changes in the balloon portion 502 of the band 504, one or more bladders 508 as disclosed herein are incorporated in the gastric band assembly. A refill port 510 is used by the doctor to inject fluid into the port which is in fluid communication with the bladders 508 and balloon 502.

Increasing the compliance of the band may actually facilitate the use of higher starting intra-band, band contact, intra-luminal pressures or smaller stoma size (diameter, area, etc.). Higher capacitance or compliance allows the band and stoma diameter to increase more readily in response to higher intra-luminal pressures generated by the esophagus during swallowing. Even the starting pressure in this case may be higher and the corresponding stoma diameter may be smaller because it takes less esophageal energy (pressure and time) to do the work to cause it to open further to allow a bolus to pass through. A condition in which there is a higher basal intra-luminal or contact pressure, but generated by a very compliant band with large capacitance, may actually be better tolerated and exert less stress or load on the esophagus and therefore lead to less dysfunction and or dilatation.

It is also important to note that elasticity, or the ability of the band/stoma to dilate, but also quickly recover to its resting or previous state, is also an important characteristic that should be imparted by the greater capacitance or compliance. The band should allow the stoma to widen and narrow elastically or reversibly with each bolus of food that passes through. This elasticity may be important to the preservation of esophageal function and structure over time. The stoma diameter and pressures should recover quickly between swallows so that it mimics a natural sphincter in its opening and closing characteristics.

In one embodiment of the invention, one or more bladders as disclosed herein is incorporated in an existing LAGB system to increase the capacitance or compliance of the system. Even when the starting stoma size is small and the intra-band pressure is high, the stoma size can increase more readily in response to bolus pressure. In other words, it takes less bolus pressure or energy to cause a given increase in stoma size. Thus, it is easier for food to pass through initially or in response to secondary contractions. Mechanistically, fluid can flow out of the band and into the bladders with much less increase in intra-band pressure than would be seen without the bladders. Thus, it takes less energy, generated by the esophagus, to push the fluid out of the band thereby increasing the stoma size and decreasing resistance to bolus passage. Importantly, the capacitance imparted by the bladders is elastic so that after the pressure transient associated with bolus transit through the stoma subsides, the fluid is pushed back into the band by the bladders to restore the initial state. Because swallowing during eating is not an isolated single event it is important that the band, bladders, and stoma size be restored back to the initial basal state quickly before the next swallow.

The benefit of this feature is that bands can be adjusted to higher pressure or smaller stoma size with less chance of bolus obstruction or obstructions that can't be cleared. In doing so bands may be more effective in inducing satiety in patients while simultaneously being more effective in reducing episodes of bolus obstruction.

The bladders of the present invention allow the starting intra-luminal and/or contact pressures to be relatively high. Ideally, the intra-luminal pressures would be at least as high as the upper end of the range reported in the literature as corresponding to the Green Zone, i.e. 15-35 mmHg. However, the intra-luminal pressures could be higher than the upper limits or thresholds that were reported with conventional gastric bands, i.e., greater than 35 mmHg. The upper limit of intra-luminal pressure might be the peak esophageal swallowing pressure that can be generated or as high a level as possible which would not lead to esophageal dilatation or dysfunction. This might be as much as normal esophageal peak pressures of 100-120 mmHg or so.

Adjusting or initial titration of bands may become easier. Some patients don't reach satiety before the band becomes too restrictive and leads to vomiting and reflux. For some other patients there is a very narrow window of adjustment level that is difficult to achieve and maintain. Allowing higher pressures or greater band fill levels to be tolerated without vomiting and reflux potentially widens the so-called Green Zone for patients. There is a larger range of fill volumes that the patient can tolerate and once the Green Zone is found the patient/bands remain there longer before needing additional adjustment.

Incorporating the increased capacitance provided by the bladders effectively allows the bolus filling of bands, as reported by Kirchmyer in 2005, but without the accompanying complications that were reported. There could be a cost savings associated with LAGB which would make the procedure more attractive.

In one embodiment, one or more bladders are incorporated into a gastric band assembly and have a compliance that provides a basal intra-luminal or contact pressure anywhere in the range from more than 35 mmHg to 150 mmHg. More typically, the bladders would have a compliance that provides a basal intra-luminal or contact pressure anywhere in the range from 35 mmHg to 80 mmHg. Even more typically, the bladders would have a compliance that provides a basal intra-luminal or contact pressure anywhere in the range from 35 mmHg to 65 mmHg. Thus, by way of example, a bladder used in conjunction with a gastric band provides a basal intra-luminal or contact pressure in the range from 35 mmHg to 65 mmHg.

As set forth herein, the basal intra-luminal or contact pressure and the basal intra-band pressure are related. In order to achieve the high basal intra-luminal or contact pressure as disclosed (e.g., greater than 35 mmHg to 150 mmHg), the basal intra-band pressures must be relatively higher. For example, for the Realize Band®, the basal intra-band pressure can be adjusted to be anywhere in the range from 40 mmHg to 150 mmHg in order to provide a high basal intra-luminal or contact pressure range such as greater than 35 mmHg to 150 mmHg. Similarly, the basal intra-band pressure of the Lap-Band AP® can be adjusted to be anywhere in the range from 40 mmHg to 180 mmHg in order to provide a high basal intra-luminal or contact pressure range such as greater than 35 mmHg to 150 mmHg.

The high basal intra-luminal and contact pressures provided by the bladders of the present invention are at the upper end of the reported Green Zone pressure or substantially higher than the Green Zone pressures. In other words, the bladders of the present invention operate in the Red Zone as described in the literature and which the prior art authors have uniformly cautioned against operation at such high pressures.

The range of basal intra-luminal and contact pressures generated by the bladder and the balloon portion of the gastric band are higher than those disclosed in the prior art and considered optimal for weight loss. In fact, the present invention basal intra-luminal and contact pressures are in the so-called Red Zone, which the prior art authors consider much too high and the cause of patient discomfort. These higher basal intra-luminal and contact pressures can be achieved with the bladders disclosed herein because the bladders are compliant and allow the bolus of food in the esophagus to pass the band area easily as fluid rapidly exits the balloon and fills the compliant bladders. Thus, any of the following basal intra-luminal or contact pressure ranges can be achieved using any of the disclosed bladders.

Basal Intra-Luminal or Contact Pressure Range
greater than 35 mmHg
35 mmHg to 180 mmHg
35 mmHg to 150 mmHg
35 mmHg to 80 mmHg
35 mmHg to 65 mmHg

Basal Intra-Luminal or Contact Pressure Range
35 mmHg to 55 mmHg
40 mmHg to 180 mmHg
40 mmHg to 150 mmHg
40 mmHg to 90 mmHg
40 mmHg to 80 mmHg
40 mmHg to 65 mmHg
45 mmHg to 180 mmHg
45 mmHg to 150 mmHg
45 mmHg to 90 mmHg
45 mmHg to 80 mmHg
45 mmHg to 75 mmHg
45 mmHg to 70 mmHg
45 mmHg to 65 mmHg
50 mmHg to 180 mmHg
50 mmHg to 150 mmHg
50 mmHg to 80 mmHg
50 mmHg to 70 mmHg
50 mmHg to 65 mmHg
60 mmHg to 180 mmHg
60 mmHg to 150 mmHg

Basal Intra-Luminal or Contact Pressure Range
60 mmHg to 85 mmHg
60 mmHg to 80 mmHg
60 mmHg to 75 mmHg
65 mmHg to 180 mmHg
65 mmHg to 150 mmHg
65 mmHg to 90 mmHg
65 mmHg to 85 mmHg
65 mmHg to 80 mmHg
70 mmHg to 180 mmHg
70 mmHg to 150 mmHg
70 mmHg to 100 mmHg
70 mmHg to 90 mmHg
70 mmHg to 85 mmHg
75 mmHg to 180 mmHg
75 mmHg to 150 mmHg
75 mmHg to 100 mmHg
75 mmHg to 95 mmHg
75 mmHg to 90 mmHg
80 mmHg to 180 mmHg
80 mmHg to 150 mmHg

Basal Intra-Luminal or Contact Pressure Range

80 mmHg to 105 mmHg
80 mmHg to 100 mmHg
80 mmHg to 95 mmHg
85 mmHg to 180 mmHg
85 mmHg to 150 mmHg
85 mmHg to 110 mmHg
85 mmHg to 105 mmHg
85 mmHg to 100 mmHg
90 mmHg to 180 mmHg
90 mmHg to 150 mmHg
90 mmHg to 115 mmHg
90 mmHg to 110 mmHg
90 mmHg to 105 mmHg
95 mmHg to 180 mmHg
95 mmHg to 150 mmHg
95 mmHg to 120 mmHg
95 mmHg to 115 mmHg
95 mmHg to 110 mmHg
100 mmHg to 180 mmHg
100 mmHg to 150 mmHg

Basal Intra-Luminal or Contact Pressure Range
100 mmHg to 125 mmHg
100 mmHg to 120 mmHg
100 mmHg to 115 mmHg
105 mmHg to 180 mmHg
105 mmHg to 150 mmHg
105 mmHg to 130 mmHg
105 mmHg to 125 mmHg
105 mmHg to 120 mmHg
110 mmHg to 180 mmHg
110 mmHg to 150 mmHg
110 mmHg to 135 mmHg
110 mmHg to 130 mmHg
110 mmHg to 125 mmHg

It is possible that the basal intra-luminal or contact pressure for optimal weight loss is at or near the normal esophageal peak pressure range of 100 mmHg to 120 mmHg, which can be achieved using the bladders herein.

It is noted that when the physician adjusts a patient's gastric band, the physician adds (or removes) fluid from the assembly to set an approximate basal intra-band pressure, which will translate to an approximate basal intra-luminal and contact pressure. With the present invention bladders in the assembly, the physician preset basal intra-luminal or contact pressure falls within any of disclosed ranges in order to reduce or eliminate adverse events (e.g., vomiting, bolus obstructions, etc.) and achieve improved rate of weight loss.

### GERD

The present invention LAGB-plus-bladder configuration can benefit patients having gastroesophageal reflux disease (GERD). It is well known that back flow of gastric contents into the esophagus results when gastric pressure is sufficient to overcome the pressure gradient that normally exists at the gastro-esophageal junction (GEJ) or when gravity acting on the contents is sufficient to cause retrograde flow through the GEJ. In order to reduce the likelihood of GERD in a patient, the present invention bladder system can be said to have a high intra-luminal or contact pressure, anywhere in the range from 30 mmHg to 150 mmHg, which will exert substantial pressure in closing the stoma. In other words, when gastric pressure is elevated the likelihood of backflow past the GEJ is substantially reduced because the stoma is being forced closed by the LAGB-plus-bladder configuration having a high intraluminal pressure. By way of example only, a contact pressure in the range from 50 mmHg to 80 mmHg will provide substantial pressure on the stomach thereby reducing the stoma diameter by an amount sufficient to block the backflow of gastric contents into the esophagus. Even at this pressure, however, a food bolus will pass through the stoma because of the compliance of the bladder allowing fluid to transfer from the balloon to the bladder during the swallow, and then fluid transferring back to the balloon after the bolus of food has passed through the stoma. Thus, the present invention bladder assembly in conjunction with an LAGB can be set at sufficiently high intraluminal or contact pressures in order to treat GERD.

Distensibility vs. Compliance

In the context of this disclosure, LAGB band or system (i.e., LAGB plus one or more bladders) compliance refers to the rate of basal intra-band pressure change per unit change in band/system fill volume (i.e., ΔBP/ΔBV). In contrast, LAGB band or system distensibility refers to the rate of band contact dimensional change per unit change in applied band-to-stomach contact pressure (i.e., ΔSD/ΔSP), usually under the assumption of a constant total band/system fill volume. Band contact dimension could be band contact diameter, band contact circumscribed area, or any other relevant dimensional description of this band contact.

While distensibility and compliance share some interdependence, they are indeed distinct characteristics of the band/system. Compliance functionally relates to the relative strength with which the band/system resists the infusion of additional fill volume. This additional fill volume imparts an internally-sourced isobaric hydrostatic pressure within the band/system that is equally opposed by the elastically-deformable band/system as it accommodates that additional volume. In this context, it is assumed that the band/system will elastically-deform into a physical configuration (e.g., shape, volume distribution, etc.) that represents its lowest viable energy state for that given fill volume. This resistance is generally measured via intra-band pressure; and therefore, compliance can be quantified as ΔBP/ΔBV.

Distensibility functionally relates to the relative strength with which the band/system resists the application of additional band-to-stomach contact pressure. This additional contact pressure imparts an externally-sourced net-outwardly-radial force to the band's balloon that causes its physical configuration (e.g., shape, volume distribution, etc.) to deform away from its lowest viable energy state for that given fill volume. This change in physical configuration is generally measured via band-to-stomach contact dimension (e.g., diameter, circumscribed area, etc.); and therefore, distensibility can be quantified as ΔSD/ΔSP. Perhaps more simply, compliance describes the ability/challenge of deforming the band/system to its lowest-energy physical configurations (as a function of total fill volume), whereas distensibility describes the ability/challenge of deforming the band/system away from these lowest-energy physical configurations. These challenges are not necessarily equivalent or proportional—that is, knowing one does not necessarily enable a complete description of the other.

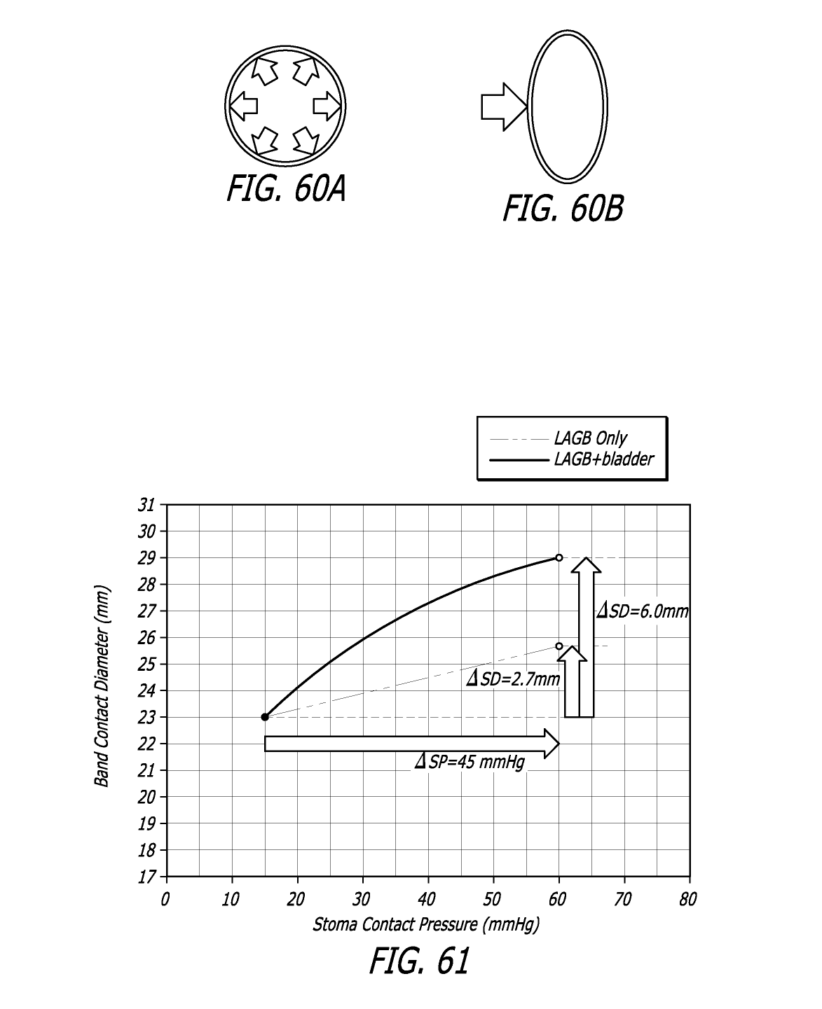

By way of simple analogy, consider an elastic sphere (e.g., a water balloon). As solution is infused into the sphere as shown in FIG. 60A, the sphere expands symmetrically, since this circular cross-sectional shape represents its lowest energy level for the given fill volume (i.e., requires the least amount of overall stretch of the sphere's outer shell). In contrast, if this sphere is then acted upon by a directed external force (FIG. 60B), that sphere deforms from that spherical shape into an ellipsoidal shape.

### System and Method for Increasing Distensibility of an LAGB

As illustrated in the series of representative examples below, adding a system of one or more passive compliant bladders that are separate from, yet in continuous direct fluid communication with, the LAGB balloon increases the effective distensibility of the LAGB contact dimension.

A series of in-vitro bench experiments were performed to explore and determine the distensibility characteristics of LAGBs alone and LAGBs connected to a bladder of the present invention. The results from these discrete in-vitro experiments were then analyzed to develop continuous mathematical functional descriptions of the inter-relationships between (a) total fill volume (abbreviated BV below), (b) intra-band pressure (BP), (c) band-to-stomach contact diameter (SD) or area (SA), and (d) band-to-stomach contact pressure (SP). The graphs described infra were derived in-silico from these mathematical relationships. The “band contact dimension” (e.g., diameter, circumscribed area, etc.) refers to the amount of stomach tissue encircled by the balloon portion of the gastric band, measured by diameter, circumscribed area, or another dimension.

The series of in-vitro bench experiments were conducted to evaluate the distensibility characteristics of LAGBs alone and LAGBs connected to a bladder system. The set-up consisted of the band portion of the selected LAGB secured around a modified EndoFLIP impedance planimetry balloon (Product Ref EF-325; Crospon, Inc.; with a 35-mm diameter replacement balloon). For each targeted step in LAGB or LAGB-plus-bladder system total fill volume, the EndoFLIP balloon (the “stomach”) was first initialized with sufficient volume to establish a maximal band-to-stomach contact pressure (generally 50-60 mmHg), and then the EndoFLIP balloon was slowly evacuated via a syringe pump until the measured contact pressure dropped below 5 mmHg. Basal intra-band pressure (BP), band contact diameter (SD), and band contact pressure (SP) were all simultaneously acquired/recorded during each fixed-volume run (SD via the EndoFLIP system; BP and SP via an HP Pressure Monitor with M1006A modules; all acquired using a National Instruments USB-6009 DAQ hardware and a custom LabView program).

The dashed curve in FIG. 61 illustrates the representative distensibility of a band contact diameter in response to a 45-mmHg increase in contact pressure—from a basal level of 15 mmHg (ref Burton's intra-luminal Green Zone pressure) to a peak level of 60 mmHg. In this example, the LAGB was an Ethicon SAGB-VC (Realize Band-C) filled with ˜9.0 mL of total fill volume, therein generating a basal intra-band pressure of ˜46 mmHg and a basal band contact diameter of ˜23.0 mm. As the contact pressure was increased to 60 mmHg, the band's contact diameter expanded to ˜25.7 mm, for a net distension of ˜2.7 mm. While the applied contact pressure increased by 45 mmHg during this distension, the intra-band pressure increased by ˜77 mmHg (peaking at ˜122 mmHg not shown.)

In contrast, when a bladder of the present invention is attached to this LAGB, the band's distensibility is notably increased as compared to the LAGB-only configuration, as illustrated by the solid curve in FIG. 61. In this LAGB-plus-bladder configuration, the system was filled with ˜17.5 mL of total fill volume, which established equivalent basal intra-band pressures and band contact diameters of ˜46 mmHg and ˜23.0 mm, respectively. Yet, for the same 45-mmHg increase in contact pressure, the band's contact diameter was able to expand to ˜29.0 mm, for a net distension of ˜6.0 mm. Also of interest (but not shown), in this configuration, the intra-band pressure peaked at only ˜81 mmHg, for a net intra-band pressure increase of ˜35 mmHg.

In summary and as illustrated in FIG. 61, for the same 45-mmHg increase in applied contact pressure (i.e., from 15 to 60 mmHg), the addition of a bladder to an LAGB more than doubled the band's contact diameter distensibility (i.e., LAGB-plus-bladder ΔSD=˜6.0 mm vs. a nominal LAGB-only ΔSD=˜2.7 mm). An analogous result was observed when distensibility was quantified with respect to band contact area or any other band contact dimensional metric (not shown).

Furthermore, these effects are not unique to the SAGB-VC LAGB; for example, similar effects were observed in-vitro and in-silico when adding a bladder to an Allergan Lap-Band AP Standard LAGB.

This increased distensibility provides opportunities for improved methods of using an LAGB. As shown in FIG. 61, this increased distensibility enables the band contact dimension of the LAGB-plus-bladder configuration to open to a substantially larger dimension (vs. a LAGB-only configuration) for any given increase in applied contact pressure (e.g., as generated by the esophagus during swallowing). In this way, the band is potentially able to successfully accommodate larger (or otherwise more challenging) food boluses within a person's swallowing capability without obstructing or inducing other obstructive symptoms.

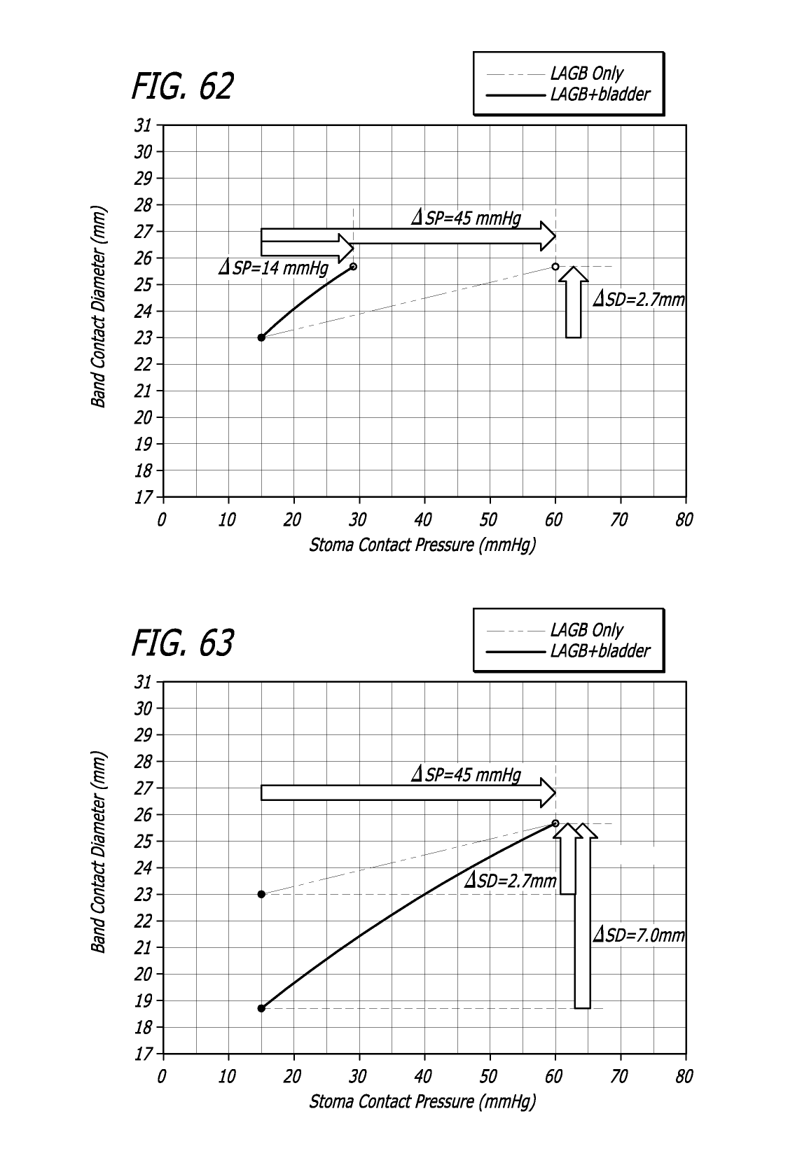

Alternatively, and as illustrated in FIG. 62, this increased distensibility enables the band contact dimension of the LAGB-plus-bladder configuration to open by a given amount with substantially less required increase in contact pressure (i.e., swallowing pressure transient). Since it is postulated that very high swallowing pressures (even if the swallow is ultimately successful) might induce adverse effects such as pouch dilatation, this embodiment could enable successful swallowing while reducing the possibility of pouch dilatation or other adverse effects. In the example illustrated in FIG. 62, the LAGB-only and LAGB-plus-bladder systems were set to the same basal conditions as those described in FIG. 61. Thus, the LAGB-only curve here (dashed curve in FIG. 62) is identical to that in FIG. 61, wherein ΔSP=45 mmHg was required to open the band contact diameter by ΔSD=2.7 mm. In contrast, with the LAGB-plus-bladder configuration (solid curve in FIG. 62), only ΔSP=14 mmHg was required to open the band contact diameter by the same ΔSD=2.7 mm.

In another alternative method of use, this increased distensibility enables the band contact dimension of the LAGB-plus-bladder configuration to be set to a tighter basal dimension and still be opened to the same final band contact dimension for any given increase in applied contact pressure. This embodiment is illustrated in FIG. 63. As above, the basal conditions for the LAGB-only configuration (dashed curve) were identical to those used previously (i.e., ˜9.0 mL of total fill volume and basal contact pressure of 15 mmHg, therein generating a basal intra-band pressure of ˜46 mmHg and a basal band contact diameter of ˜23.0 mm). In contrast, while the basal contact pressure was identical (15 mmHg), the total fill volume for the LAGB-plus-bladder configuration (solid curve) was increased to ˜22.5 mL, which resulted in a basal intra-band pressure of ˜78 mmHg and a basal band contact diameter of ˜18.7 mm. Yet when acted on by an equivalent ΔSP=45 mmHg, the LAGB-plus-bladder configuration was able to successfully open to the same peak band contact diameter (SD=25.7 mm) as that achieved by the LAGB-only configuration. Thus, this example suggests that a LAGB-plus-bladder system would be able to similarly accommodate a food bolus of a given maximal dimension from a tighter basal condition compared to that possible with an LAGB-only system. This “tighter basal condition” can be achieved by filling the system with additional total fill volume beyond “nominal,” setting the system to a higher basal intra-band pressure beyond “nominal,” etc. It has been postulated that satiety signaling is enhanced at tighter band settings, thus this ability in the LAGB-plus-bladder configuration to set the band to a tighter basal dimension may further improve the positive therapeutic effects while not negatively impacting/increasing the negative adverse effects.

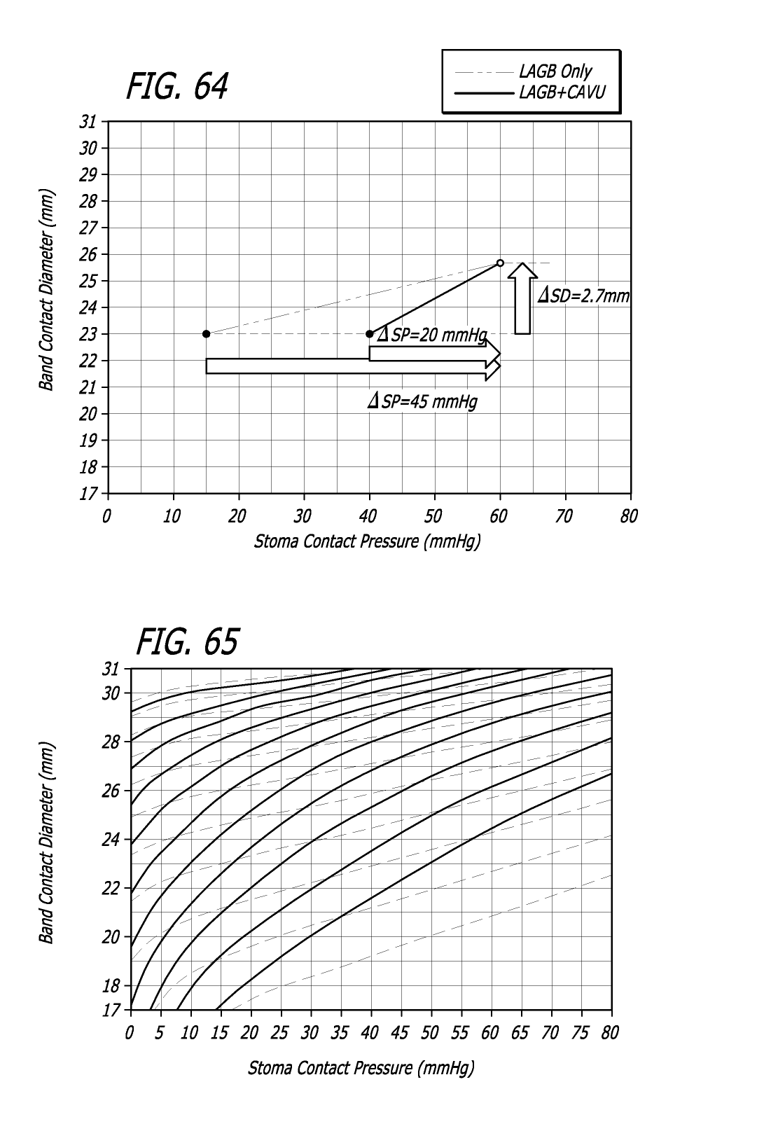

In yet another alternative method of use, this increased distensibility enables the band contact dimension of the LAGB-plus-bladder configuration to accommodate a higher basal contact pressure for given target basal and peak contact dimensions and a given peak contact pressure. This embodiment is illustrated in FIG. 64. As above, the basal conditions for the LAGB-only configuration (dashed curve) were identical to those used previously (i.e., ˜9.0 mL of total fill volume and basal stoma contact pressure of 15 mmHg, therein generating a basal intra-band pressure of ˜46 mmHg and a basal band contact diameter of ˜23.0 mm). While the total fill volume of the LAGB-plus-bladder system was the same as that used in the example of FIG. 63 (22.5 mL), the applied basal contact pressure was increased to 40 mmHg, resulting in a basal intra-band pressure of ˜101 mmHg and a basal band contact diameter of ˜23.0 mm. Nevertheless, the band contact diameters from these LAGB-only and LAGB-plus-bladder configurations were both able to successfully expand to 25.7 mm (ΔSD=2.7 mm) at an absolute peak contact pressure of 60 mmHg.

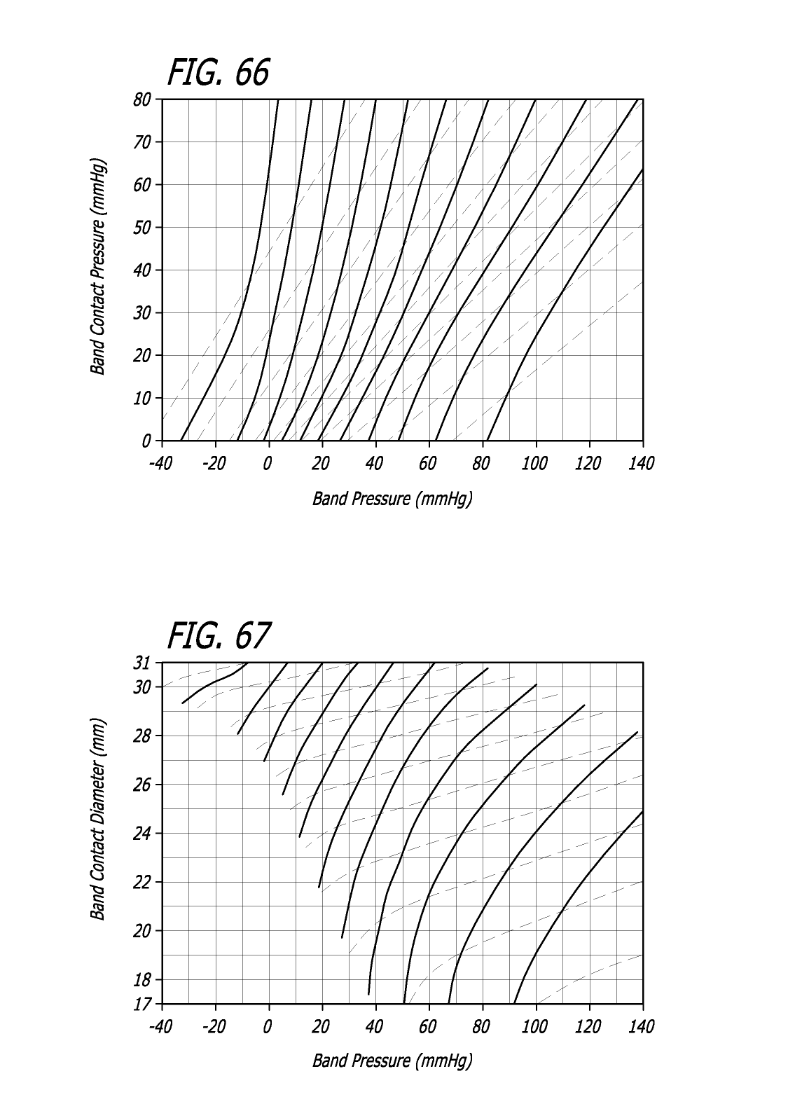

The examples described above provide only representative examples from an otherwise continuous parameter space encompassing all viable combinations of total fill volume, intra-band pressure, band contact dimension, and band contact pressure. FIGS. 65-67 provide further insight into the inter-relationships across this entire parameter space. These representative curve sets were derived from the in-silico model of the SAGB-VC LAGB alone (dashed curves) or in combination with a bladder system (solid curves). Each curve traverses the continuous parameter surface at a constant total fill volume, ranging from 2-12 mL for the LAGB-only system (1-mL steps) and 4-24 mL for the LAGB-plus-bladder system (2-mL steps). All points on this parameter surface (not just the points along these curves) are viable combinations of total fill volume, intra-band pressure, band contact dimension, and band contact pressure. Furthermore, the exact path of this surface through parameter space may be affected by one or more internal and/or external forces, such as: temperature, stress relaxation of the system's components (e.g., silicone balloons and/or bladders), LAGB choice, functional compliance of the bladders, growth of fibrous tissue over some or all of the system components, etc. Therefore, these examples and Figures are intended to provide representative illustration of the principles and advantages of enhanced distensibility afforded to an LAGB via the addition of a system of one or more passive compliant bladders.

As mentioned in Burton, et al. (Burton P R, et al., 2009. “Effects of gastric band adjustments on intraluminal pressure.” Obesity Surgery, 19(11), p. 1508-14) in successful patients (presumably those in the Green Zone), the basal intra-luminal pressure at the level of the LAGB was consistently at or near the range of 15-35 mmHg despite patients having different bands. They further posited that the likely reason that few LAGB patients exceed a basal intraluminal pressure of 35 mmHg is that it is simply beyond the capacity of the esophagus to transit solid food across the LAGB at those elevated intra-luminal pressures.

Using in-silico models of LAGB and bladder systems, demonstrates (1) how stoma (band contact dimension) distensibility can be linked to Burton's observations, and (2) how an increase in stoma distensibility (e.g., via the addition of a bladder to the LAGB) may beneficially expand the limits of the so-called Green Zone.

Experimentally-derived in-silico mathematical models of LAGB pressure-volume-diameter relationships and bladder pressure-volume relationships were utilized for these analyses.

This study made the following assumptions:
The esophagus could generate/apply a maximum of 80 mmHg (absolute) of opening force to the band contact dimension during a swallow (i.e., peak contact pressure);
Time-dependencies (if any) were not limiting factors in the interactions between applied forces and system responses, and thus would have had minimal/negligible impact on the observed results (or resultant conclusions) if they had been included.

The primary question explored through this study was:
Over a range of possible combinations of basal intra-band pressure and basal contact pressure, how much will the band contact dimension (i.e., area or diameter) open during a swallow (with a peak absolute contact pressure as defined above)?

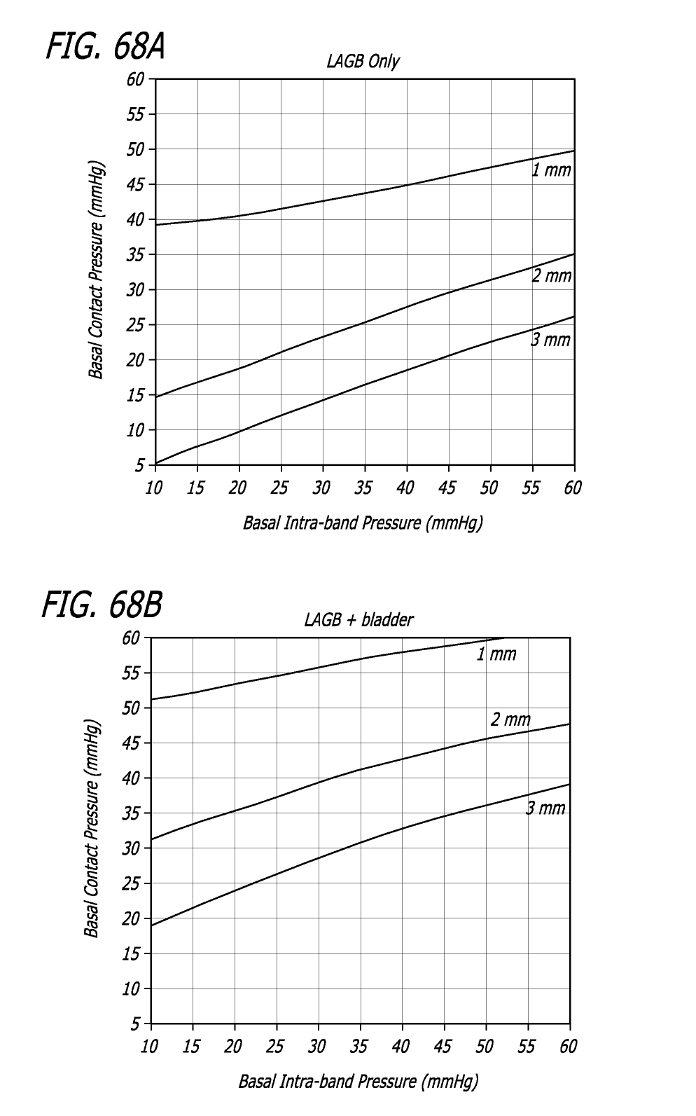

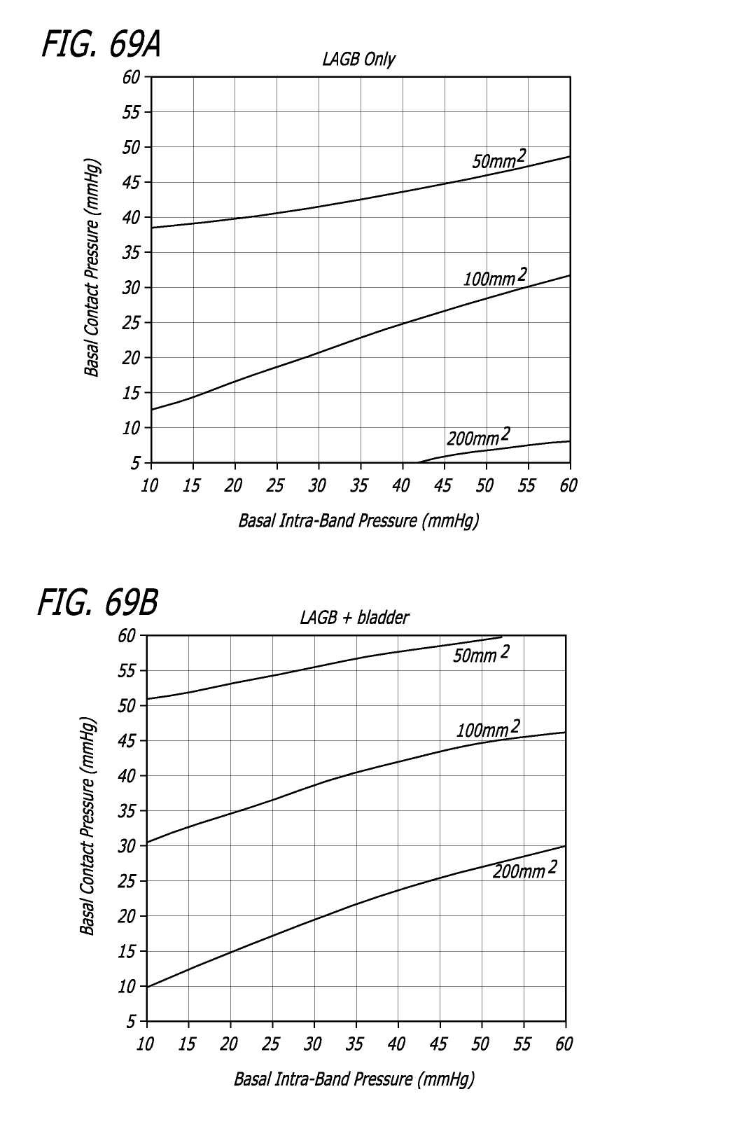

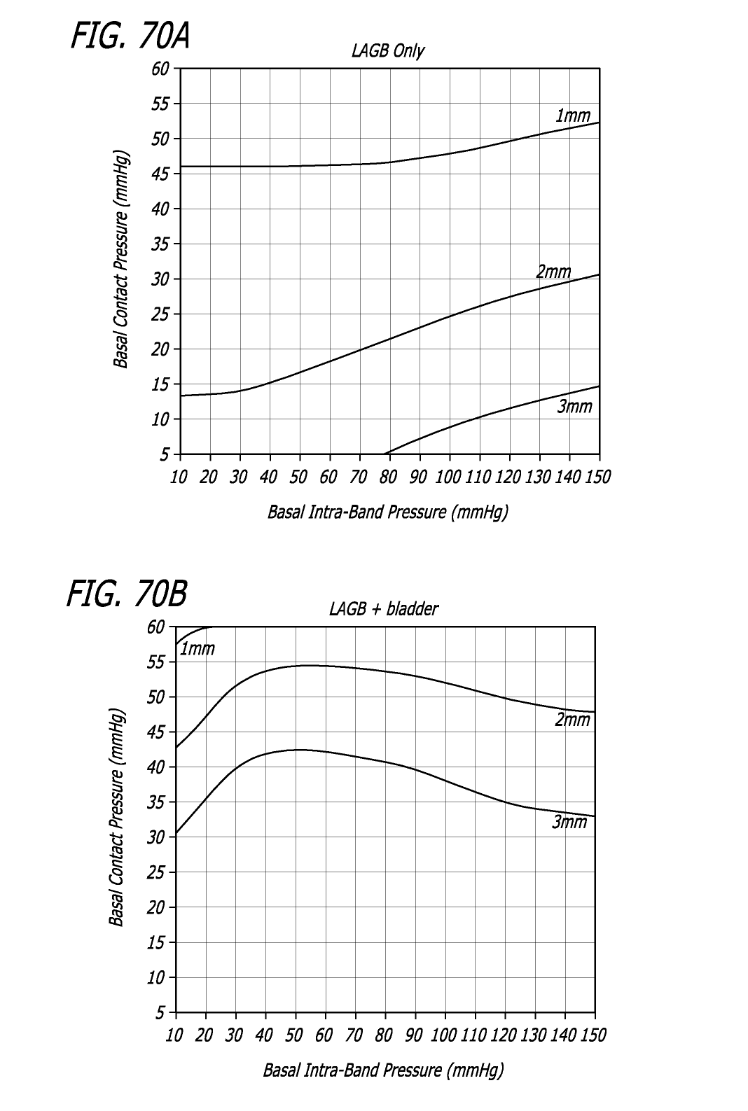

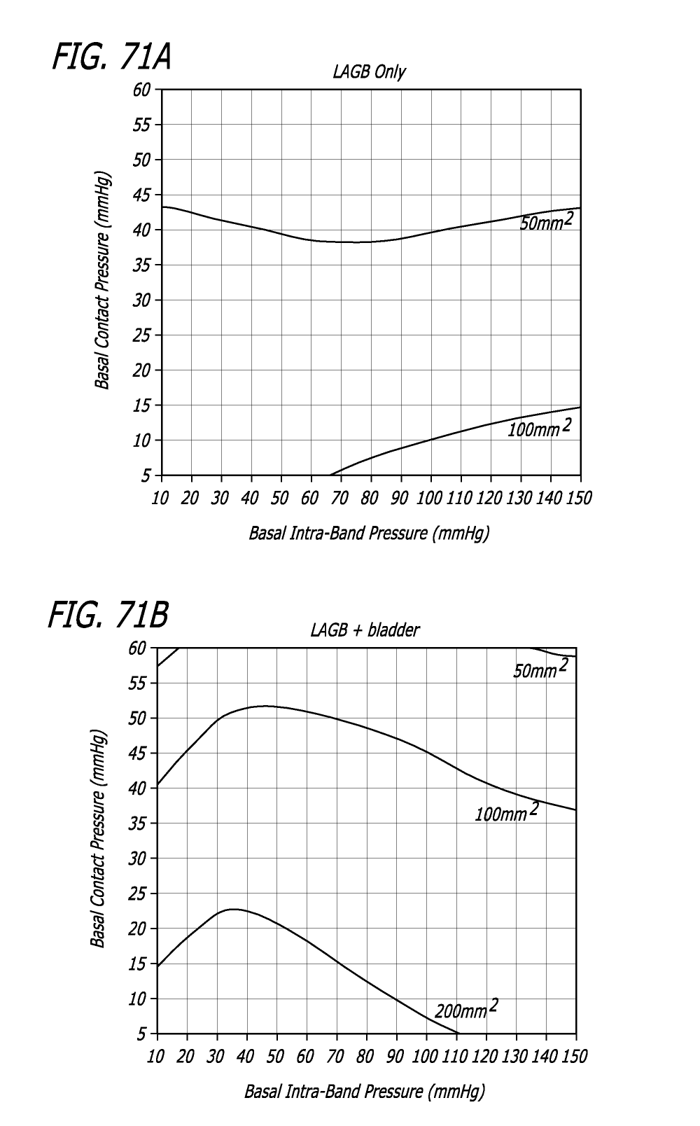

Associated in-silico experiments were run for the Ethicon SAGB-VC LAGB and the Allergan Lap-Band AP Standard LAGB in both an LAGB-only configuration and an LAGB-plus-bladder configuration. Dimensional changes in band contact size were quantified both via net changes in band-to-stomach contact diameter (ΔSD) and net changes in band-to-stomach circumscribed contact area (ΔSA). Contour plots of ΔSD and ΔSA derived from the results obtained across the associated ranges of basal intra-band pressures and basal contact pressures are provided in FIGS. 68A-69B (SAGB-VC) and FIGS. 70A-71B (APS) (see figures for ranges and increments used). A few selected contour lines are highlighted within each otherwise-continuous contour surface.

One approach to interpret the contour plots of FIGS. 68A-69B is as follows: Assume that a person swallows a bolus of food, and that the bolus requires the band contact area to open by at least ΔSA≧100 mm² (with respect to its basal size) in order for the bolus to successfully pass through without obstructing. If this person's esophagus can only generate a maximum of 80 mmHg of absolute opening pressure to the band contact area, then this person will have a successful swallow when their basal conditions fall within the regions of the ΔSA contour plots in which ΔSA≧100 mm² (i.e., “below” the ΔSA=100 mm² contour line), while this person will obstruct when their basal conditions fall within the regions of the ΔSA contour plots in which ΔSA<100 mm² (i.e., “above” the ΔSA=100 mm² contour line). With respect to operating basal intra-band pressure and basal contact pressure, this ΔSA=100 mm² contour line thus represents the upper limit to the Green Zone (i.e., the transition line between the Green Zone and the Red Zone). Note that, for any chosen transition level, this Green Zone is substantially expanded for the LAGB-plus-bladder configuration as compared to the associated LAGB-only configuration. In other words, because the addition of the bladder system to an LAGB substantially increases the distensibility of the band contact area in response to a given “swallow” input, the person is able to more easily achieve a successful swallow across a broader range of basal intra-band pressures and basal contact pressures. Conversely, the person is less likely to obstruct.

Another approach to interpret the contour plots of FIGS. 68A-69B is as follows: Assume that a person swallows a bolus of food. For any specific combination of basal intra-band pressure and basal contact pressure, these contour plots identify the maximal stoma size increase that is achievable given a maximum of 80 mmHg of absolute opening pressure. A point-by-point comparison between the contour plots from the LAGB-only and LAGB-plus-bladder configurations reveals that the LAGB-plus-bladder configuration always achieves a larger stoma size increase. In other words, because the addition of the bladder system to an LAGB substantially increases the distensibility of the stoma in response to a given “swallow” input, the person is able to more easily achieve a successful swallow for larger food boluses than could be achieved with the LAGB alone. Accordingly, the person with the LAGB-plus-bladder configuration is less likely to obstruct. Analogous interpretations are achievable via analyses of the ΔSD contour plots (e.g., ΔSD≧2 mm).

Similar relationships are achieved for different maximal opening pressures, threshold levels, etc. Thus, these conclusions are not specific to the particular values chosen for these examples.

The degree of added distensibility afforded to the LAGB with the addition of a bladder of the present invention is dependent on the particular pressure-volume characteristics of that bladder(s). These examples were generated using only one specific PV embodiment (per LAGB) of these bladder systems, but obviously these results can be easily modulated via appropriate changes to the associated PV profiles.

After an LAGB is implanted around a patient's stomach, that LAGB generally requires periodic adjustments to its total fill volume in order to attain/maintain the desired therapeutic outcome (e.g., weight loss) while minimizing any adverse effects (e.g., obstruction, vomiting, etc). Bands that are properly adjusted within this therapeutic “sweet spot” are considered to be in the Green Zone. Bands that are under-filled (insufficient therapy) are said to be in the Yellow Zone while Bands that are over-filled (excessive adverse effects) are said to be in the Red Zone. (Ref Burton P R, et al., 2009. “Effects of gastric band adjustments on intraluminal pressure.” Obesity Surgery, 19(11), p. 1508-14.)

Fill volume adjustments effectively result in a concomitant adjustment to the band contact size—increasing total fill volume results in a relative reduction in (narrowing of) the band contact size, while decreasing total fill volume results in a relative increase in (opening of) the band contact size.

The interaction between the LAGB and the encompassed stomach tissue occurs via (and can be quantified by) the band-to-stomach interfacial contact pressure (i.e., band contact pressure or contact pressure). The “interfacial contact pressure” is defined as the pressure at the contacting interface between the LAGB balloon and the outer surface of the encompassed stomach tissue. It is believed that the encompassed stomach tissue changes its effective dimension (e.g., thickness) in response to the LAGB-applied contact pressure through one or more mechanisms. For example, the encompassed stomach tissue might temporarily increase in effective thickness due to swelling/edema, irritation, etc (more common soon after LAGB implantation). Conversely, the encompassed stomach tissue might decrease in effective thickness due to progressive remodeling of underlying fat/tissue, dispersal of underlying fluids/blood, etc. These dimension-reducing processes will continue until the interfacial contact pressure drops to a level that no longer drives further change (i.e., an equilibrium is reached). If this equilibrium contact pressure results in an intra-luminal stoma dimension that now permits foods to pass too easily and/or reduces the associated satiety signaling (equivalent to an intra-luminal pressure that now falls within the Yellow Zone), then that patient will no longer enjoy adequate therapy from their LAGB. At this point, an incremental fill volume adjustment is necessary to tighten the LAGB so as to re-engage the encompassed stomach tissue and therapeutically ‘reposition’ the LAGB within the Green Zone.

It is known that, with current LAGB systems, it generally requires several incremental fill adjustments (especially during the first several months after LAGB implantation) in order to reach a patient's “Green Zone plateau” wherein an adequate and sustained therapeutic effect is achieved and maintained between and across follow-up visits without the need for additional (or significant) fill adjustments. Additionally, patients often describe that, during this filling phase, they might “feel great” immediately after an adjustment (i.e., adjusted back into the Green Zone), but then that therapeutic benefit quickly diminishes over the next few days or weeks or so (i.e., falls back into the Yellow Zone), presumably as the encompassed stomach tissue progressively remodels due to the elevated contact pressure. Thus, there exists an opportunity to improve LAGB therapeutic potential/robustness by (a) reducing the total number of incremental fill adjustments in order to reach a patient's “Green Zone plateau,” and/or (b) improving the preservation of LAGB therapy as the encompassed stomach tissue responds to any fill volume increment (e.g., tissue remodeling).

It is also known that these LAGB systems are relatively sensitive to the amount of incremental volume delivered to/from the LAGB—that is, the Green Zone is relatively narrow with respect to fill volume, thus making it relatively easy to over- or under-fill the LAGB and thereby resulting in Red Zone or Yellow Zone (respectively) outcomes. This sensitivity is thought to be due to the relatively steep relationship between the induced contact pressure and changes in LAGB contact size (the latter of which, as mentioned above, is modulated through changes in total fill volume). Thus, there exists a related opportunity to (c) improve LAGB therapeutic performance by improving the preservation of contact pressure despite any fill-modulated changes in band contact dimension.

Experimentally-derived in-silico mathematical models of LAGB pressure-volume-diameter relationships and bladder pressure-volume relationships were utilized for these analyses.

The analyses presented in this embodiment assume that tissue remodeling will occur when the interfacial contact pressure between the band and the encompassed tissue (i.e., band contact pressure) exceeds a particular positive magnitude. In this scenario, the encompassed tissue will progressively decrease in dimension until that interfacial contact pressure reaches ˜10 mmHg; at this pressure, an equilibrium is assumed to have been reached and no further remodeling occurs. Other equilibrium values could have been assumed without any loss of generality with respect to the observed results and inferred conclusions. This value is generally consistent with Burton's research (Burton, et al, 2009) that suggests that the transition between “Yellow” and “Green” Zones occurs at an intra-luminal pressure of approximately 15 mmHg. These analyses also assume that time-dependent changes (if any) were not limiting factors in the interactions between applied forces and system and/or tissue responses, and thus would have had minimal/negligible impact on the observed results (or resultant conclusions) if they had been included.

### Contact Pressure Preservation Despite Tissue Remodeling

As illustrated in the representative examples below, adding a system of one or more passive compliant bladders that are separate from, yet in continuous direct fluid communication with, the LAGB balloon increases the ability to (a) better preserve LAGB-tissue interfacial contact pressures despite any ongoing contact-induced tissue remodeling, and as a consequence (b) induce a greater amount of tissue remodeling for a given contact pressure potential.

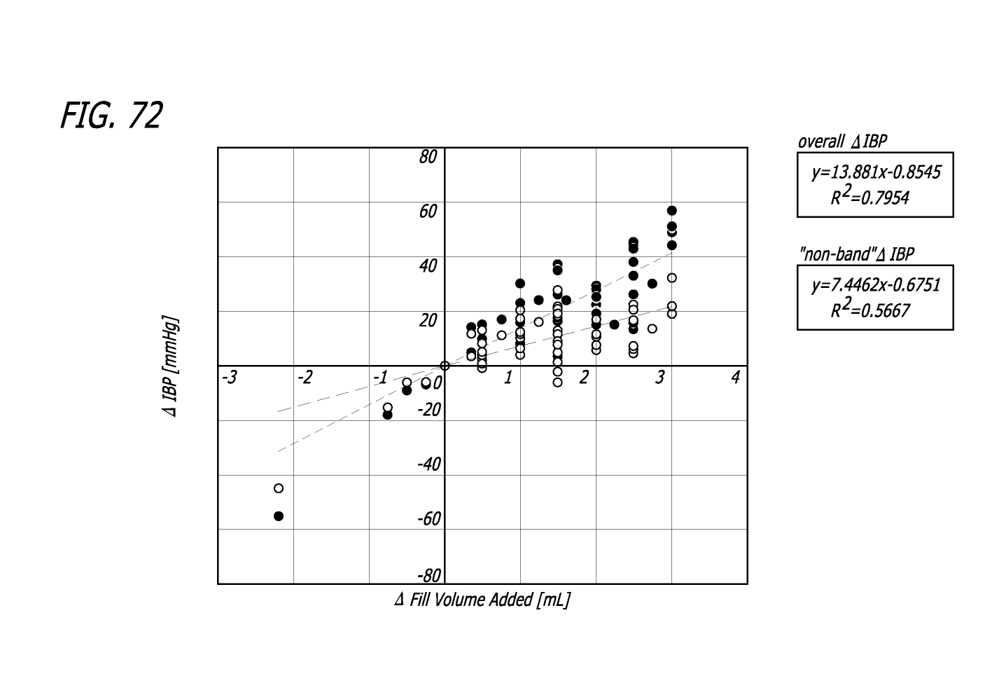

As mentioned in the Background section supra, the potential energy that drives progressive tissue remodeling is thought to come from the elevated interfacial contact pressure established with each fill volume increment. Data from ongoing (LAGB-only) human clinical study indirectly suggest that this fill-induced step increase in contact pressure is on the order of 5-20 mmHg. FIG. 72 presents a scatterplot of LAGB Δpressure (ΔIBP) vs. LAGB Δvolume (ΔFill) pairings as measured from SAGB-VC LAGB-implanted study subjects during each of their follow-up visits. The solid dots indicate that, on average, overall LAGB pressure increased by ˜14 mmHg for every 1 mL added to the LAGB. However, this measured overall ΔIBP represents the superposition of two main contributors: ΔIBP from stretching the LAGB balloon itself, and ΔIBP from increased interfacial contact pressure. The ΔIBP from LAGB stretch alone can be estimated using the unconstrained PV relationships determined from in-vitro bench measurements. By subtracting off that component, it is then possible to estimate the residual ΔIBP from increased contact pressure alone. These “non-band” ΔIBP values are plotted in FIG. 72 as circles (unshaded) from which it can be inferred that the interfacial contact pressure increased by ˜7.5 mmHg for every 1 mL added to the LAGB. Since the incremental fills for these SAGB-VC LAGBs ranged in magnitude from 0.5-3.0 mL (median and mode 1.5 mL), the associated fill-induced incremental increase in interfacial contact pressure is inferred to range from ˜4 to ˜22 mmHg (median ˜11 mmHg). In the representative example infra, a fill-induced increase in interfacial contact pressure of 10 mmHg is assumed. Other values could be evaluated with equal ease.

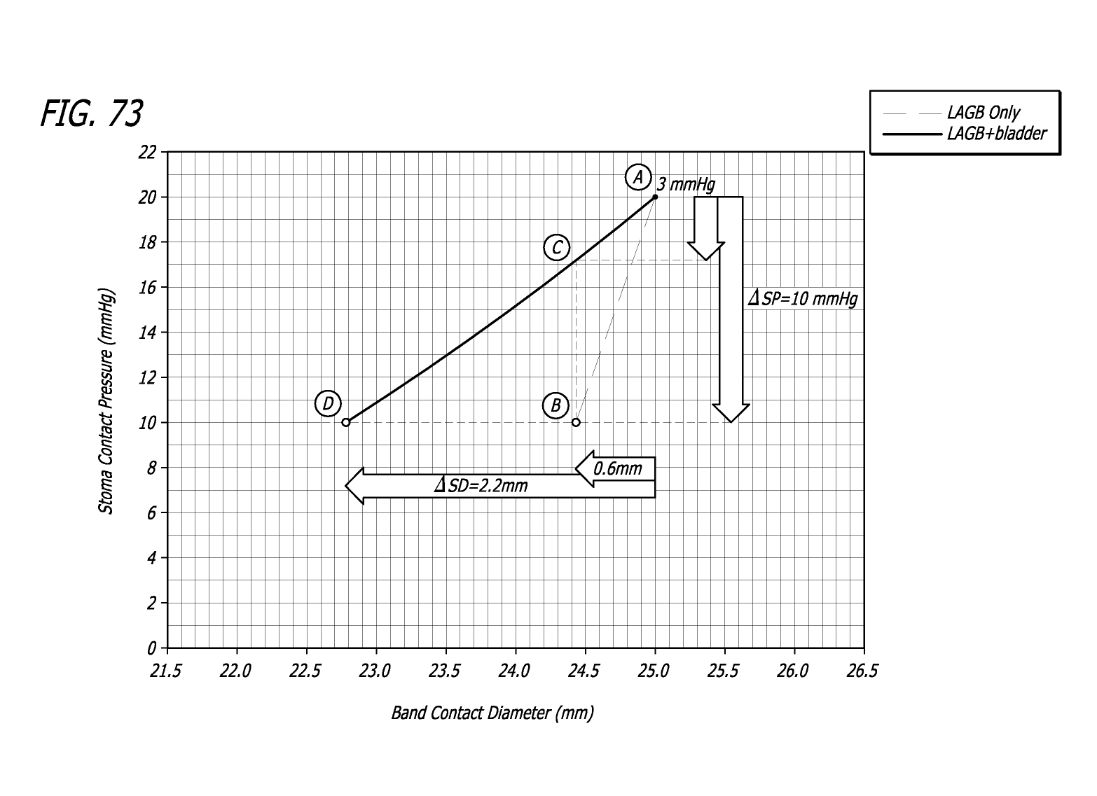

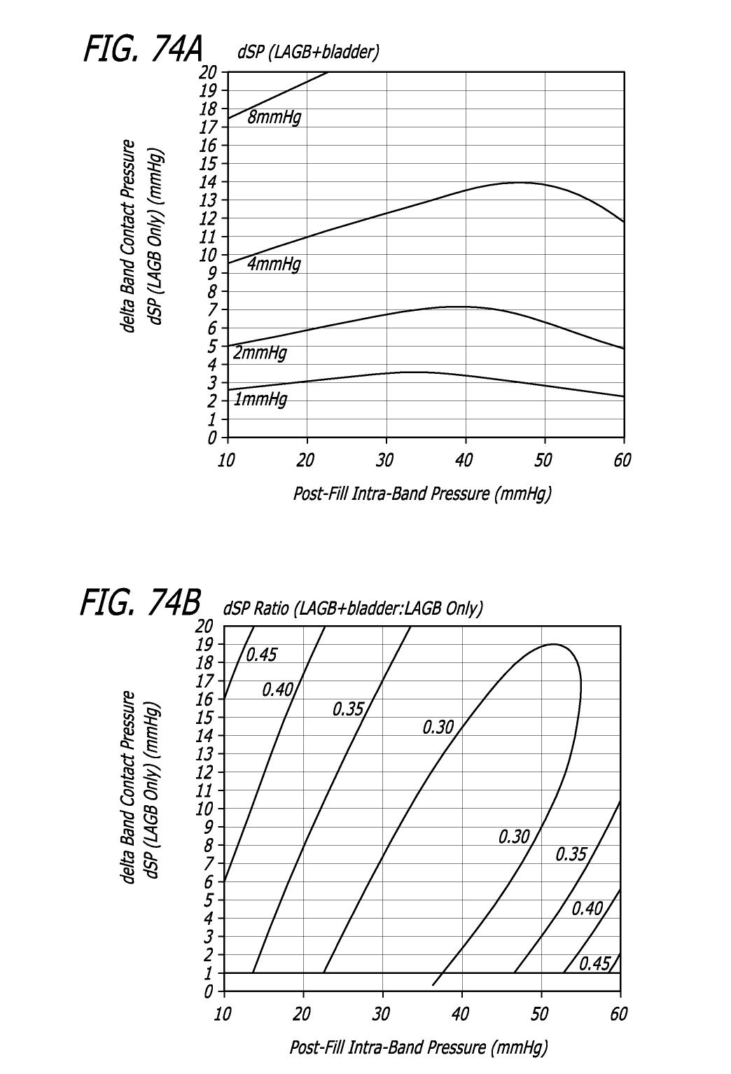

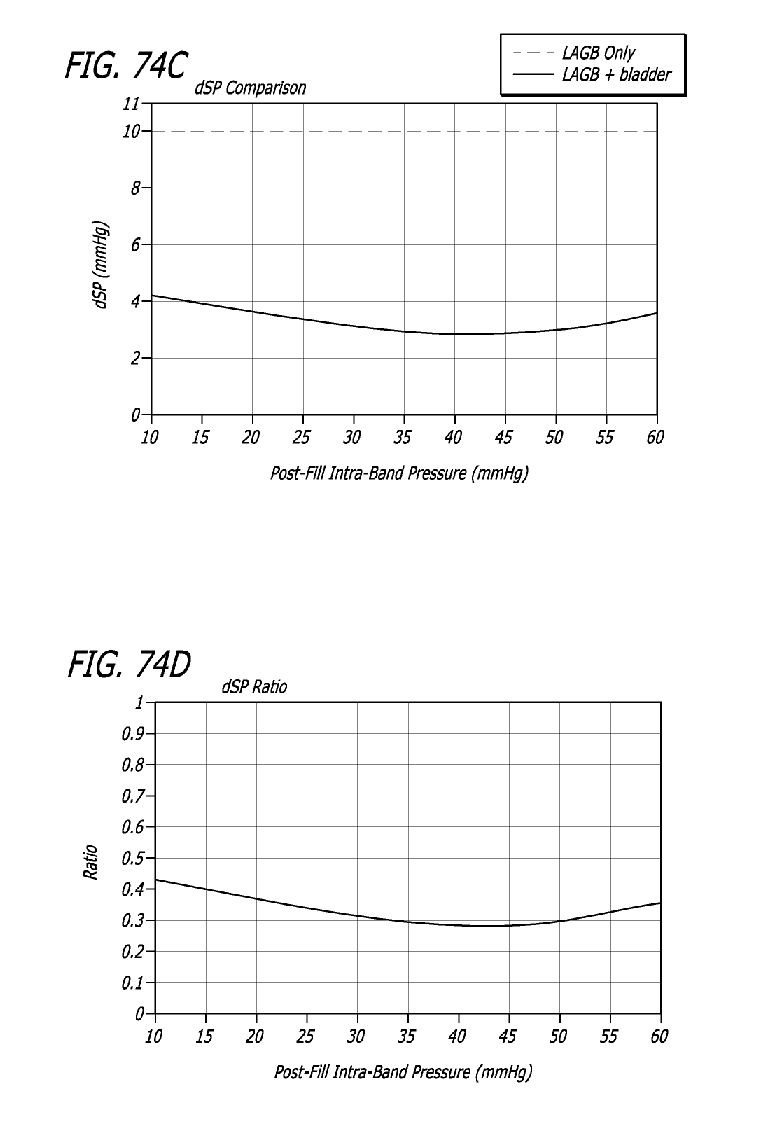

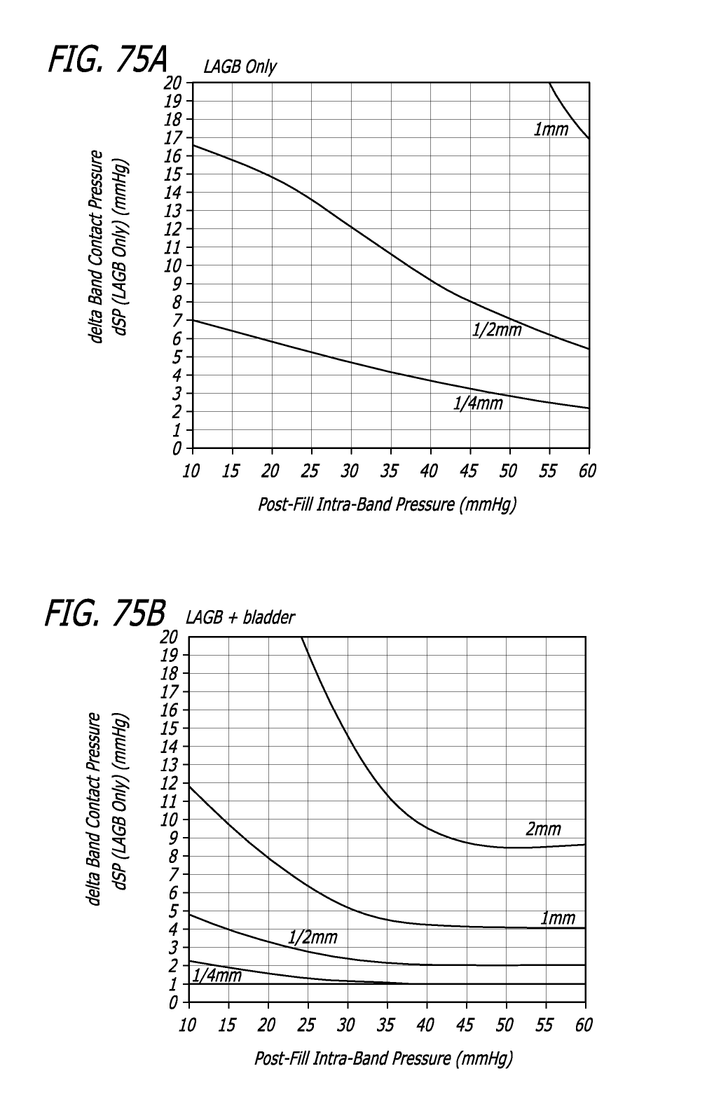

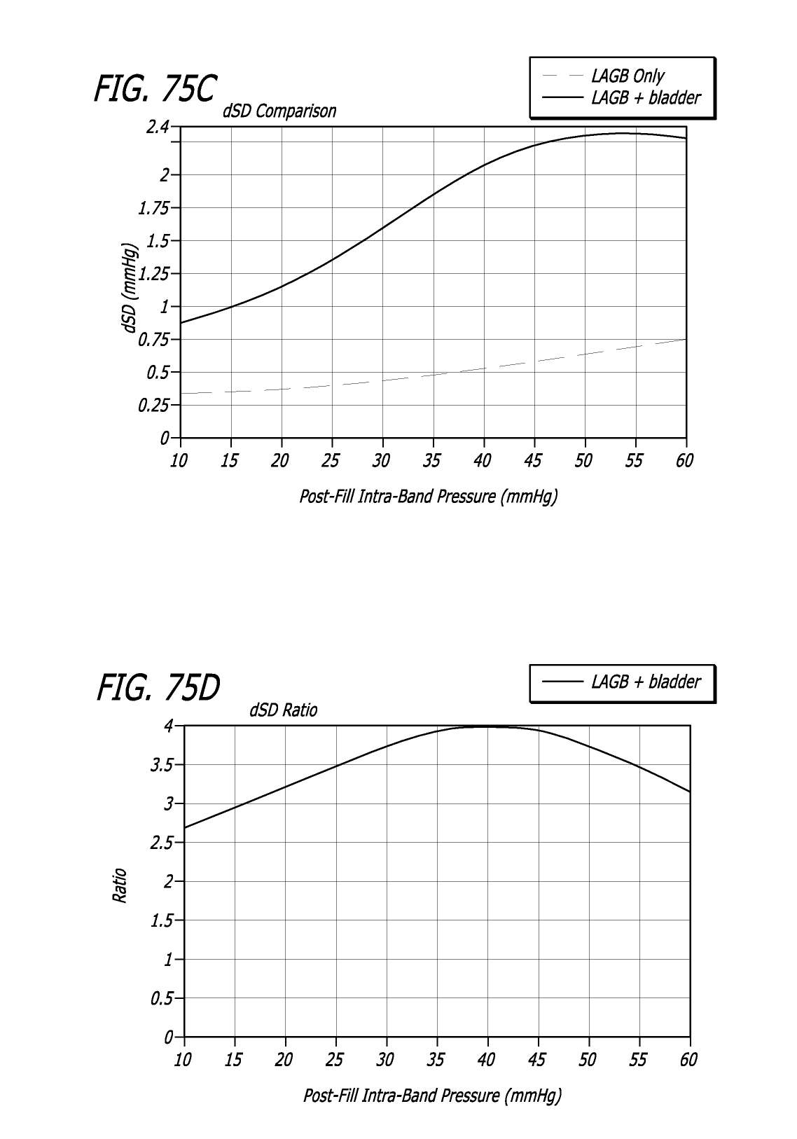

In the example presented in FIG. 73, it is imagined that a patient attends a follow-up appointment at which they receive an incremental fill volume adjustment to their LAGB (SAGB-VC) that results in a post-fill band-to-stomach contact diameter (SD) of 25.0 mm and an interfacial contact pressure (SP) of 20 mmHg (labeled point A in FIG. 73). Since the new interfacial contact pressure is greater than the assumed remodeling equilibrium threshold of 10 mmHg, there exists a net potential energy of 10 mmHg to drive remodeling and hence band contact size reduction. With an LAGB-only configuration, this potential energy is expended after only ˜0.6 mm of band contact diameter reduction (labeled point B in FIG. 73). However, with an LAGB-plus-bladder configuration, the SP has only decreased to ˜17 mmHg (ΔSP=˜3 mmHg) after an equivalent reduction in band contact diameter (labeled point C in FIG. 73). Thus, the LAGB-plus-bladder configuration has effectively preserved ˜7 mmHg of SP potential energy that can continue to drive further band contact size reductions. In fact, this LAGB-plus-bladder configuration is estimated to result in a total ΔSD of ˜2.2 mm for the same A SP of 10 mmHg (labeled point D in FIG. 73). For reference, but not shown, the associated intra-band pressures at labeled points A to D were estimated as: (A) ˜44 mmHg, (B) ˜29 mmHg, (C) ˜42 mmHg, (D) ˜36 mmHg.

The results exemplified in FIG. 73 represent the outcome for a particular starting condition (e.g., total fill volume or post-fill intra-band pressure) and driving contact pressure (e.g., ΔSP above equilibrium threshold). The first contour plot of FIG. 74A summarizes the ΔSP for a LAGB-plus-bladder configuration (equivalent to the SP difference between point A and point C in FIG. 73) across a range of post-fill intra-band pressures (x-axis) and LAGB-only ΔSP's (y-axis; equivalent to the SP difference between point A and point B in FIG. 73) for the SAGB-VC LAGB. FIG. 74B maps the computed ratios of LAGB-plus-bladder A SP's to LAGB-only ΔSP's across these same x- and y-axes. Note that these ratios are substantially less than one for all starting conditions, illustrating that the addition of the bladder system to an LAGB universally preserves contact pressures substantially better than is capable via the LAGB alone. FIG. 74C plots the subset of LAGB-plus-bladder ΔSP values assuming that the LAGB-only ΔSP value equals 10 mmHg (FIG. 73). FIG. 74D plots the associated LAGB-plus-bladder ΔSPs to LAGB-only ΔSP's ratios from the data of FIG. 74C.

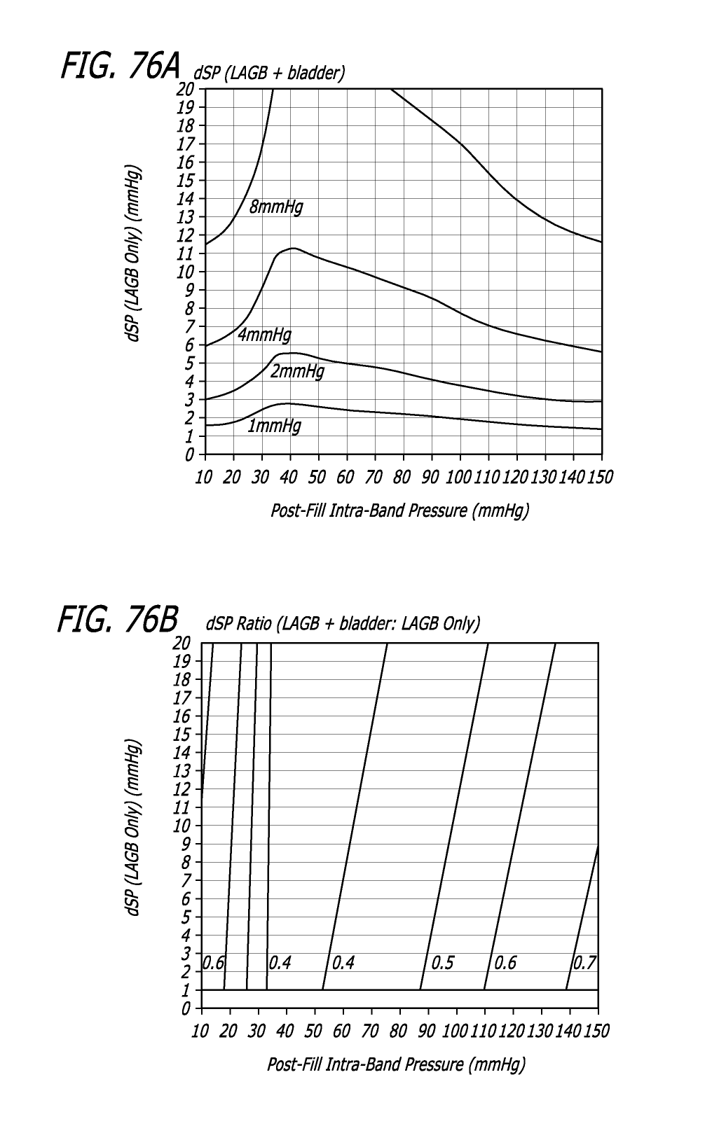

While FIGS. 74A-74D summarize the generalized comparison of contact pressure differences (ΔSP) at point B vs. point C in FIG. 73, FIGS. 75A-75D summarize the generalized comparison of band contact diameter differences (ΔSD) at point B vs. point D in FIG. 73 for the SAGB-VC LAGB. FIGS. 75A and 75B map the absolute ΔSD attained with the LAGB-only or LAGB-plus-bladder configuration, respectively, across the range of given post-fill intra-band pressures (x-axis) and driving ΔSP's (y-axis). FIG. 75C plots the subset of LAGB-only and LAGB-plus-bladder ΔSD values assuming ΔSP equals 10 mmHg. FIG. 75D plots the associated LAGB-plus-bladder ΔSD to LAGB-only ΔSD ratios from the data of FIG. 75C. Note that these ΔSD ratios are substantially greater than one for all starting conditions, illustrating that for any given driving ΔSP, the addition of the bladder system to an LAGB universally induces substantially greater band contact size reduction (e.g., remodeling) than is capable via the LAGB alone.

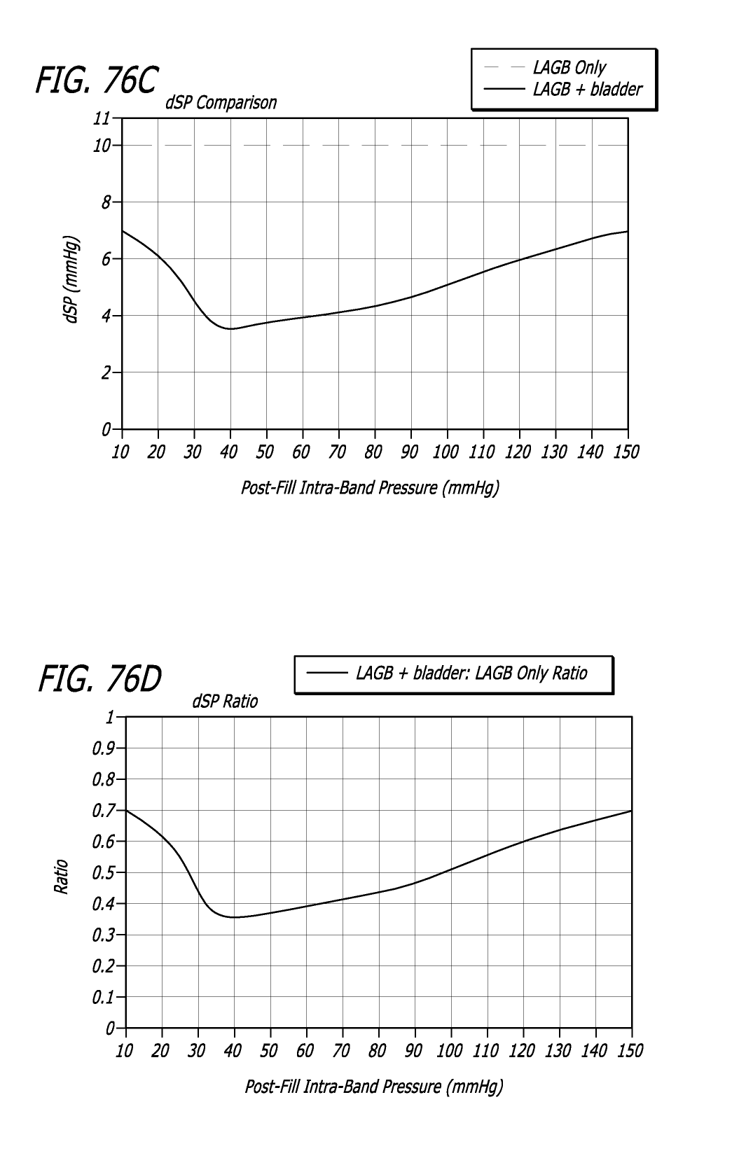

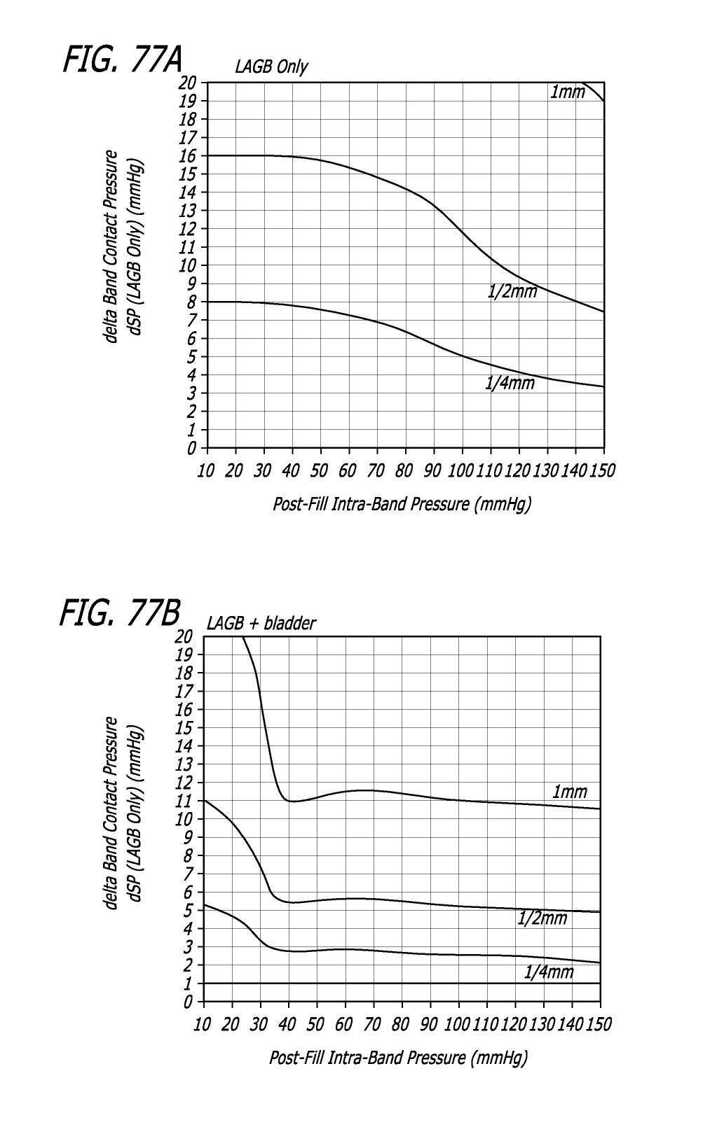

FIGS. 76A-76D and FIGS. 77A-77D present the equivalent results as that presented in FIGS. 74A-74D and FIGS. 75A-75D, respectively, but for the APS LAGB instead. The results are quantitatively similar; the conclusions qualitatively equivalent.

### Progressive Distensibility

Obstructive symptoms when swallowing (e.g., vomiting, productive burping, reflux, etc.) are known to be a significant issue for many LAGB patients. These symptoms become particularly prevalent and problematic if/when, e.g., the LAGB is adjusted relatively tightly, the patient attempts to swallow a relatively large and/or fibrous food bolus, etc.

One aspect of LAGB function that substantially impacts swallow success (vs. obstruction) is the relative “distensibility” of the band to enable successful transit of the food bolus passed/through the stoma encircled by the LAGB. LAGB's alone have limited distensibility; however, such distensibility can be increased substantially via the addition of a bladder to the LAGB as described in experiments supra. In the analyses of the experiments, it was explicitly assumed that the results were not time-dependent. Implied in this assumption is that the results represented “no-flow” equilibrium conditions, i.e., any pressure differentials that might have existed between any connected LAGB and/or bladder system components (and thus would have driven fluid flow down that pressure gradient) had fully equilibrated.

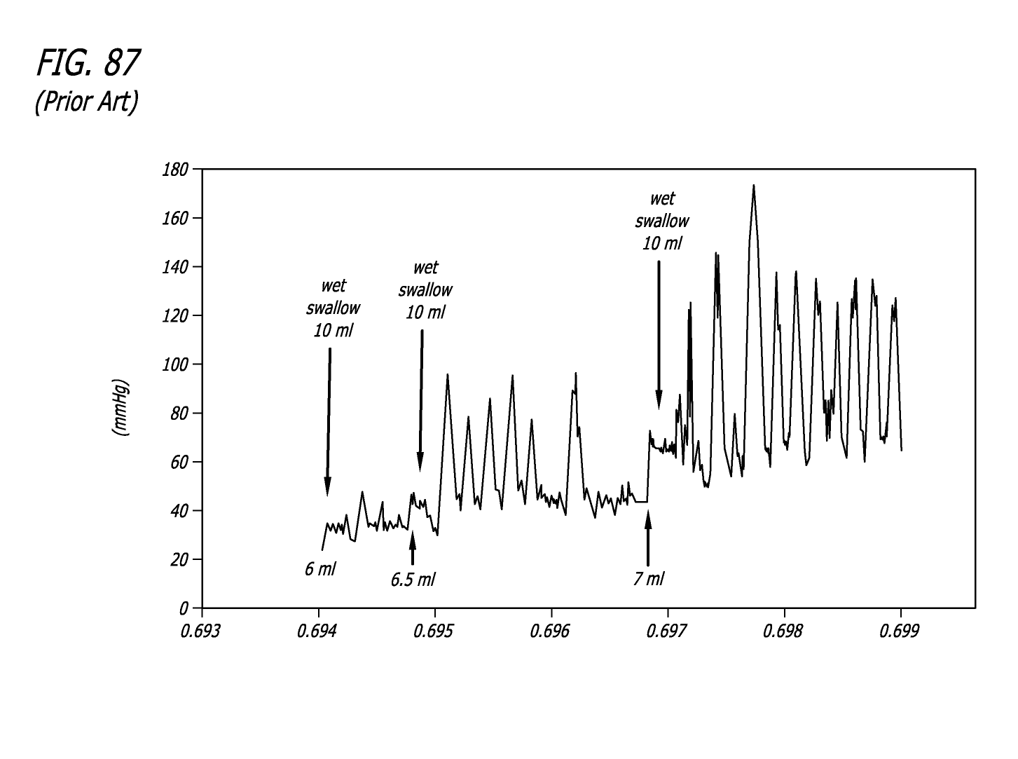

Swallowing, however, is not a steady-state action, but rather involves time-dependent processes resulting in time-dependent variations in intra-luminal pressures, stoma size, contact pressures, intra-band pressures, etc. For example, Lechner et al. (Lechner W, Gadenstätter M, Ciovica R, Kirchmayr W, Schwab G. In vivo band manometry: a new access to band adjustment. Obesity Surgery 2005; 15(10):1432-6) recorded intra-band pressures vs. time during bolus wet swallows at different volume adjustments of the LAGB (FIG. 87 Prior Art). At a LAGB volume of 6 mL, the bolus was passed with a single esophageal peristaltic wave. But as the LAGB was tightened to 6.5 mL and then 7.0 mL, this patient had increasing difficulty with passing the bolus, as evident from the multiple secondary peristalses that were observed.

The present invention explicitly considers these time-dependent aspects of swallowing, and discloses how a flow restrictor between the band and the bladder can be harnessed to enable “progressive distensibility” of the LAGB stoma in a LAGB-plus-bladder configuration. The time-dependent aspects of swallowing, referred to herein as “progressive distensibility,” incorporates a flow restrictor into an assembly having a LAGB-plus-bladder configuration. Flow restrictors were previously described in co-pending U.S. Ser. No. 12/819,443 filed Jun. 21, 2010, the entire contents of which are incorporated herein by reference. Portions of the flow restrictor application are reproduced here as support for the claims.

Over time the level of restriction in a patient varies. There are several characteristic types. There is the steady gradual loss or loosening that occurs over weeks and months. This may be due to air or saline diffusion out of the gastric band and also tissue adaptation or remodeling inside the band. Conversely the band can also gradually become too tight. There are the cyclical variations of increasing then decreasing tightness that occur over weeks and months. One example of this is the variations that correspond to menstruation. In addition, there are similar cyclical cycles of loosening and tightening that occur on a daily basis known as diurnal variations where the band is typically too tight in the morning and too loose in the evening These phenomena might be measurable by the intra-band or contact pressures in the bands. Even if pressures do not vary as suspected, the patient symptoms clearly do. Therefore the band-patient relationship is clearly a dynamic one and creates a moving target for adjustments.

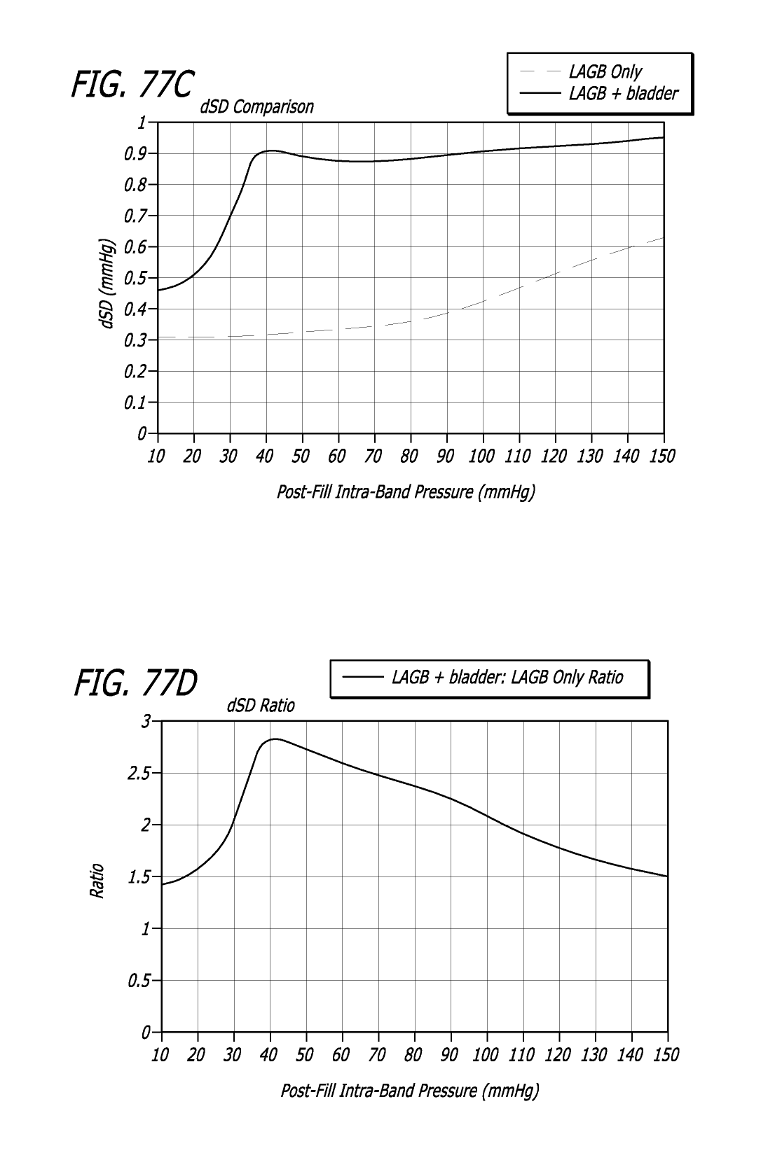

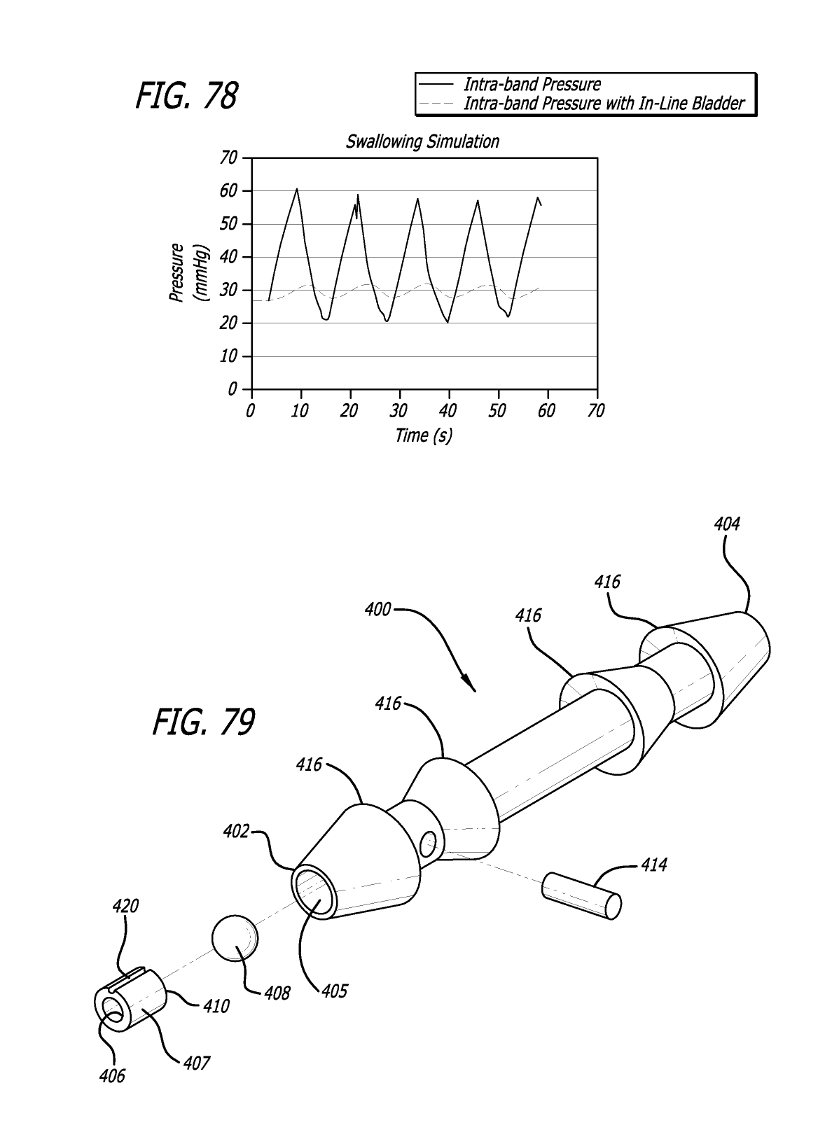

Two different mechanical states of a gastric band have been characterized; a basal resting state and a dynamic one that occurs during swallowing. As shown in the representative example of FIG. 78, the dynamic state is characterized by rapid and transient intra-band pressure spikes from the basal pressure up to significantly higher pressures and back down to the basal pressure. These are generated by esophageal pressure waves that are the normal mechanism of swallowing that induce corresponding intra-luminal stoma pressure transients that are then transmitted across the stomach tissue and then ultimately recordable as pressure transients within the band. In the example of FIG. 78, the intra-band pressure cyclically spikes during swallowing from about 20 mmHg to about 60 mmHg and back to 20 mmHg over a time period of about 10 to 15 seconds.

One way of viewing these behaviors is that they are pressure variations not only in amplitude, from basal to peak swallowing, but also in frequency (the inverse of period) or duration. For example, swallowing transients are high frequency events, occurring in the span of seconds. Diurnal variations in pressure occur over hours. Other variations can occur over the span of days and weeks. In general pressure variations, especially the low frequency ones, are undesirable in banding.

A solution to the lower frequency, longer period, pressure variations is the use of the bladders as described infra. These self-adjusting pressure bladders alter the pressure-volume compliance relationship of gastric band systems. They can accommodate changes in volume within the native band itself or to changes to the band-stomach interface without allowing pressures to change as much as they would have with just the native band. This minimizes the changes to the level of restriction. The bladders react very quickly such that pressure differentials between the band and bladders are eliminated very quickly, on the order of seconds or fractions of a second. Although this ability to adapt is highly desirable, it also has an undesirable side effect. As shown in FIG. 78, during swallowing, the bladder allows the fluid to rapidly exit the band significantly reducing the amplitude of the pressure wave measured in or generated in the lap band. This decrease in pressure wave amplitude may eliminate the feeling of satiety or restriction and hence diminish the performance of the band. In the example shown in FIG. 78, the intra-band pressure varies only about 5 mmHg during patient swallowing because fluid in the band rapidly flows to bladders and back to the band during the swallowing cycle. While it is important that pressure equilibrium be restored between the band and bladders for low frequency events, it may not be critical that it happens so quickly during patient swallowing. Low frequency events, that occur over minutes, hours or longer, may only need a bladder system that adapts on the order of minutes, hours or longer. For high frequency events such as swallowing, it may be desirable to preserve the pressure spike behavior that is normally seen without the bladders. These pressure spikes may be important for the patient to feel restriction during eating or to generate the mechanical stimulus that leads to satiety in properly adjusted bands. Preventing pressures from changing in these circumstances may undermine the effect of the band.

One embodiment provides a simple, sensor-less system component that modifies the behavior of the system. It has a specific frequency response such that slow or low frequency events are prevented from causing significant intra-band pressure changes, but high frequency events do generate pressure spikes. In effect this would be a low pass filter for fluid to flow between the band and bladders. Pressure differentials between the band and the bladders can be equilibrated slowly. This can be achieved by limiting the channel through which fluid moves between the band and bladders. This increases the fluid resistance and reduces the flow rate for a given pressure gradient. Low frequency pressure gradients that occur when pressure rises gradually in the band relative to the bladders such as during temporal variations lasting minutes, hours or more are alleviated because fluid can move to and from the band and bladders, albeit slowly. However, during quick events like a swallow, the fluid cannot move quickly enough through the narrowed channel from the band to the bladders to significantly lessen the rise in pressure seen on the band side.

Swallowing during a meal is not an isolated event but involves many episodes over a span of many minutes. With a fluid channel resistor between the band and bladders, as will be described more fully herein, the intra-band pressure spikes result in higher transient pressures on the band side of the resistor that do not get transmitted fully to the bladder's side. However, despite the short duration of the pressure spike, there is a large temporary gradient. Accordingly, some fluid does move from the band to the bladder. This occurs with each swallowing pressure spike. When the swallowing wave passes and pressures return to the basal state there is a net increase in fluid volume and pressure on the bladder side. This creates a pressure gradient in the opposite direction. The bladders try to maintain pressure equilibrium with the band so the fluid has a tendency to flow back to the band from the bladders. But, during the time between pressure peaks or swallows, the basal pressure gradient across the resistor is smaller than during swallowing so the fluid does not return as quickly to the band side. Repeated swallowing cycles would result in the net transfer of fluid from the band to the bladders resulting in less intra-band pressure being generated with each swallow. This would be especially true for lower pressure bands such as the Realize® (but may not be necessary in higher pressure bands such as Lap Bands® where basal pressure is close to peak esophageal pressures (80-100 mmHg)).

To compensate for this behavior a novel feature is to impart directionality to the fluid flow resistor. The fluid restrictor of the present invention provides the high fluid resistance to allow pressure to build up on the band side during a swallow, but then allows fluid to flow from the bladders to the band in the face of much less fluid resistance. During the high pressure spikes fluid would flow through the fluid restrictor under a larger pressure gradient. During the latent period in between pressure spikes, fluid could largely return to the band from the bladders at about the same rate because of substantially reduced flow resistance in this direction to compensate for the reduced pressure gradient and reduced duration of fluid flow back. This would allow the amplitude of the pressure spikes in the band during swallowing to be preserved and have less decay over many swallows.

Another important feature is to allow for emergency fluid removal at a reasonable rate. Occasionally patients need to have their bands loosened by removing fluid. This is usually because the patients are in extreme discomfort and distress. Thus, it is important to be able to remove fluid quickly and offer quick relief to the patient. The device should allow fluid to be evacuated from a band using normal syringes in the span of seconds to minutes. Despite the presence of the fluid restrictor, in vitro testing demonstrates that this can be accomplished with the prototype configurations that were tested as described more fully herein.

Related to this feature is the capability for the band to loosen gradually should food get stuck in the stoma. This is a very unpleasant experience for patients and can lead to many maladaptive behaviors that undermine the banding therapy. When food gets stuck in a conventional band, secondary esophageal pressure waves are generated in an attempt to push the food past the stenosis of the band. With conventional bands, the fluid in the band had nowhere to go so the band maintains its restriction and obstruction to the food. With the addition of the bladders to the system, the fluid can be displaced from the band to the bladders without a significant increase in pressure. Thus, the stoma size enlarges, reducing the obstruction to food. Food can become dislodged and pass through much easier in response to esophageal pressure waves. The addition of the fluid restrictor slows the passage of fluid from the band to the bladders, but still allows fluid flow so that as fluid leaves the balloon the balloon opening gets larger thereby permitting the stoma to get larger so food obstructions can be cleared. Thus, the fluid restrictor has the feature of preventing food from getting stuck above the band. Moreover, the bladder and the flow restrictor provide numerous other clinical benefits including mitigating pouch dilatation, band slippage, band erosion, stomach prolapse, and maladaptive eating behavior.

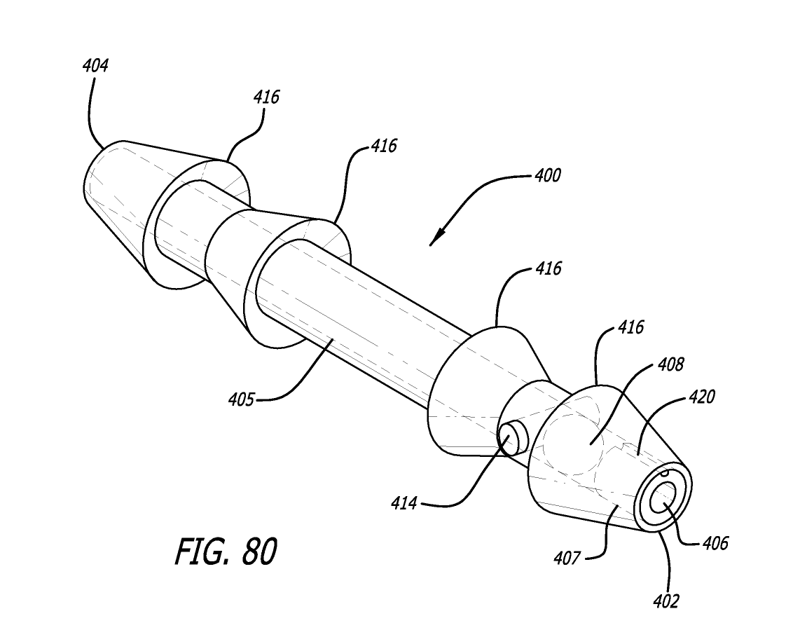

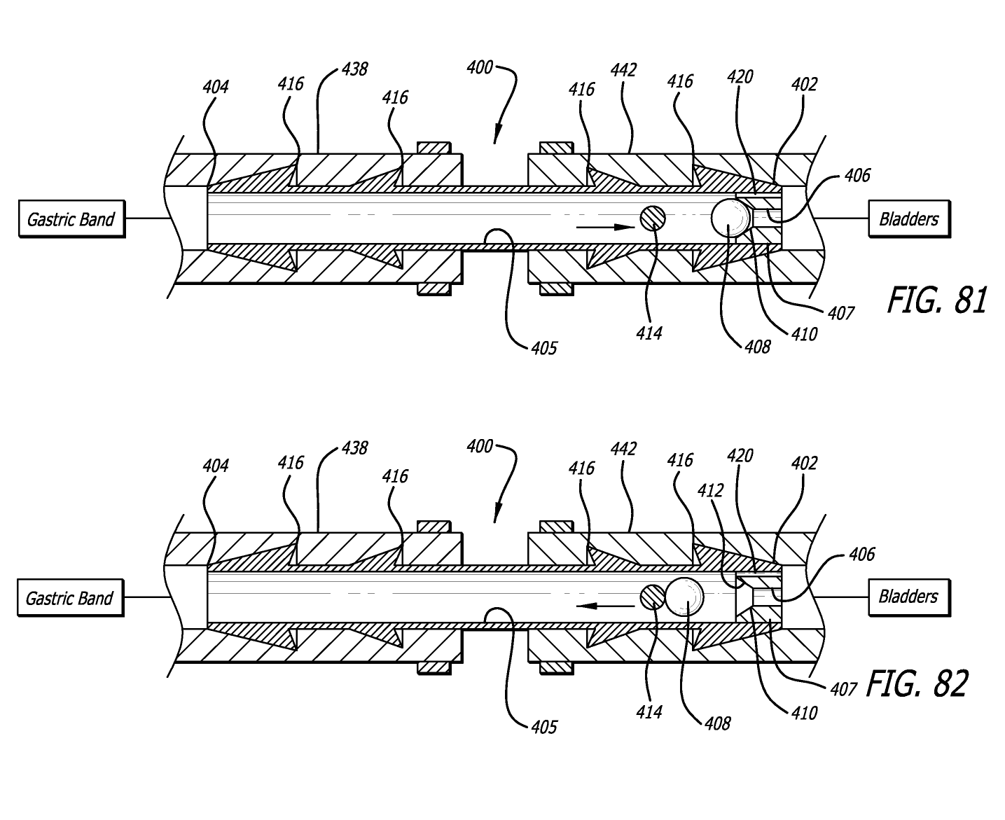

In keeping with the invention, and referring to FIGS. 79-82, a flow restrictor 400 has a distal end 404 and a proximal end 402. The flow restrictor has a fluid lumen 405 extending therethrough to permit fluid to flow in either direction through the fluid lumen. A main flow channel 406 extends through plug 407 which in this embodiment is positioned in the fluid lumen 405 at the distal end 404 of the flow restrictor 400. A non-biased ball 408 is positioned adjacent the main flow channel 406 and generally permits fluid flow through the main channel past the ball. By a non-biased ball it is meant that the ball responds very quickly in response to changes in fluid flow and direction. A ball seat 410 is formed in the plug 407 and is configured to receive ball 408. When the ball 408 is seated on the ball seat 410, fluid flow through the main channel 406 is blocked completely in the direction from the proximal end 402 through the distal end 404 of the flow restrictor 400. In this embodiment, a tapered section 412 forms the ball seat and has an angulation that is compatible with the diameter of the ball 408 so that the ball seats firmly on the tapered section 412. Alternatively, instead of tapered section 412, the ball 408 could seat on an arcuate section (not shown) having an arc that corresponds to the outer circumference of the ball. In order to prevent the ball 408 from traveling through the main channel in the proximal direction, a pin 414 is placed through the main flow channel in a transverse direction so that the ball has only limited travel movement in the main channel between the pin 414 and the ball seat 410. As shown more clearly FIGS. 79-82, ridges 416 are formed on the outer surface at the distal end 404 and the proximal end 402 of the flow restrictor 400. The ridges are configured to permit tubing to be pushed over the distal end and proximal end of the flow restrictor and the ridges 416, so that the ridges firmly attach the tubing to the flow restrictor. Ridges 416 function like barbs to firmly attach the tubing to the flow restrictor. In one embodiment, the main flow channel 406 has a diameter in the range from 0.254 mm (0.010 inch) to 6.35 mm (0.082 inch) and a length less than 76.2 mm (3.0 inch). In one preferred embodiment, the diameter of the main flow channel is 1.32 mm (0.052 inch) and it has a length in the range from 2.5 mm (0.098 inch) to 63.5 mm (2.5 inch). These dimensions, however, are exemplary and may vary depending on a number of circumstances, including the type of gastric band used, the amount of fluid volume in the gastric band assembly, and the amount of fluid flow between the gastric band and the bladders, which must flow through the fluid restrictor 400.

Still referring to FIG. 79-82, the flow restrictor 400 has a bypass channel 420 that is in fluid communication with the main channel but is positioned so that it is not blocked by the ball 408 when the ball is seated on ball seat 410. In other words, bypass channel 420 permits fluid flow in either direction through the flow restrictor at all times, and is never blocked by ball 408. The main flow channel 406 has a cross-sectional area, and the bypass channel 420 also has a cross-sectional area.

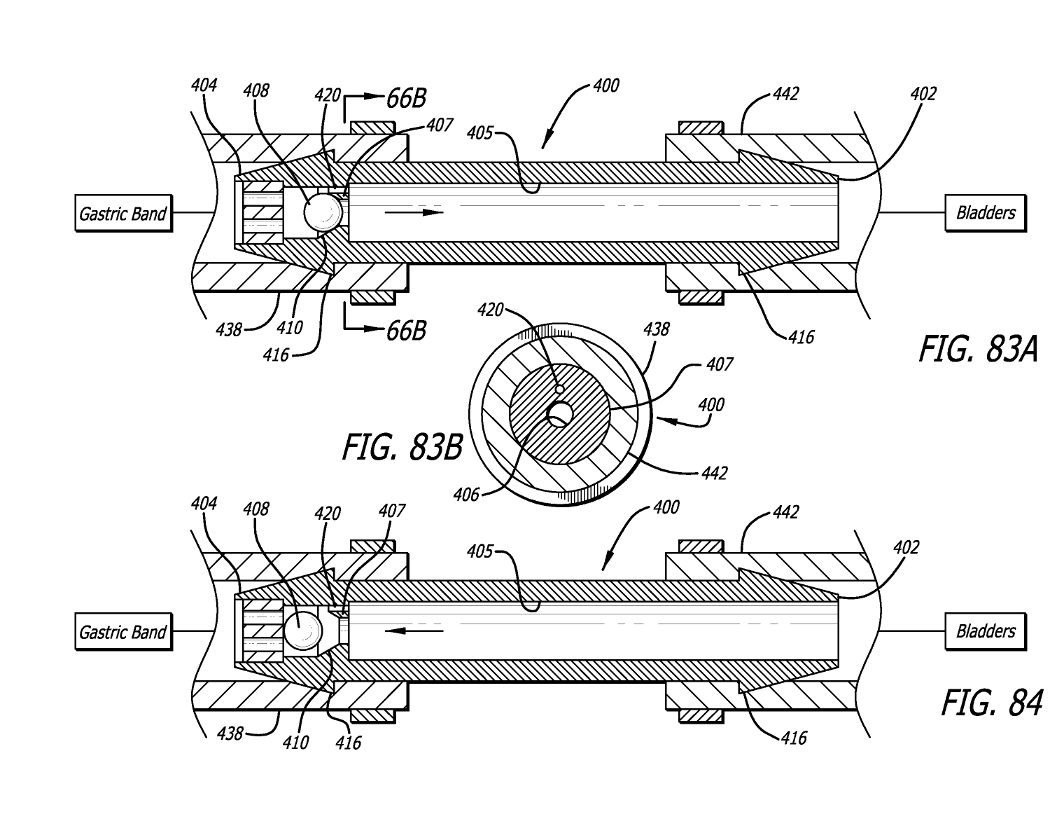

In one embodiment, as shown in FIGS. 83A-84, the flow restrictor 400 has a distal end 404 and a proximal end 402. The flow restrictor has a main flow channel 406 extending therein to permit fluid to flow in either direction through the main flow channel. A non-biased ball 408 is positioned in the main flow channel 406 and generally permits fluid flow through the main channel past the ball. A ball seat 410 is formed near the distal end of the flow restrictor and is configured to receive the ball 408. The position of the ball 408 and the ball seat 410 are at the distal end 404 of the flow restrictor, which is the opposite end from that shown in FIGS. 79-82. The operation of the flow restrictor 400 in FIGS. 83A-84 is identical to that described for FIGS. 79-82, with the exception of the location of the ball and the ball seat.

The flow restrictor 400 can be formed from any number of biocompatible materials including metals or polymers. For example, flow restrictor 400 can be formed from stainless steel, titanium, nickel titanium (nitinol), superelastic or pseudoelastic materials, or any of a number of polymer materials such as polyethylene, polyurethane, and similar materials. Further, the flow restrictor 400 can be formed from a combination of metallic, ceramic and polymer materials. The non-biased ball 408 can be made from hard materials that will resist deterioration from friction such as rubies or sapphires. Likewise, the ball seat 410 is made from a hard material such as ceramic, alumina, a coating of sapphire material, or titanium.

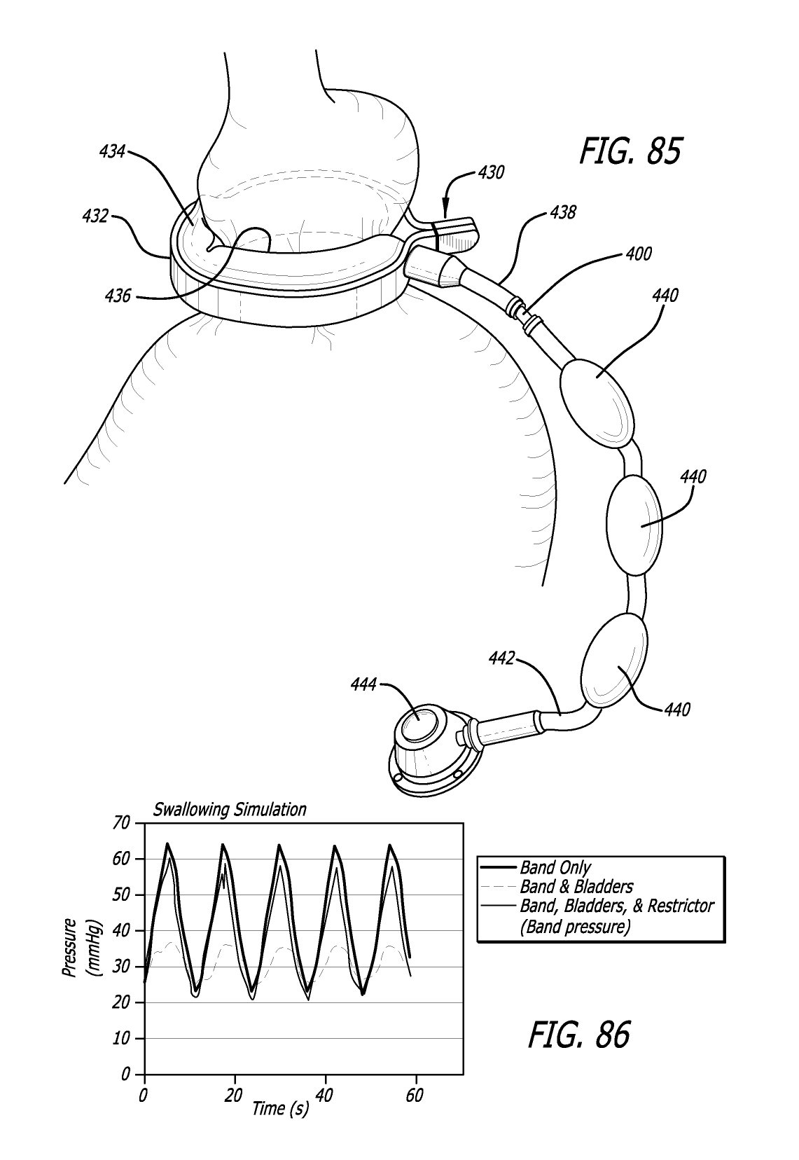

As shown more clearly in FIG. 85, the flow restrictor 400 is incorporated into a gastric band assembly 430. The gastric band assembly includes a gastric band 432 which has a balloon 434 that encircles a stoma 436, which is the stomach tissue at the top of the stomach and just below the esophagus. Tubing 438 extends from the gastric band 432 and is attached to the distal end 404 of the flow restrictor 400. As previously described, the tubing slides over ridges 416 on the outer surface of the flow restrictor and is firmly attached since the ridges have sharp edges to engage the inside of the tubing wall. The gastric band assembly 430 also includes bladders 440 such as those disclosed in FIGS. 28-60 disclosed herein. Tubing 442 extends from the bladders 440 and attaches to the proximal end 402 of the flow restrictor 400. The gastric band assembly also includes a refill port 444 as previously described herein in order to inject fluid through the port assembly and into the bladders 440. Tubing 446 extends from refill port 444 and attaches to the bladders 440. There is also tubing between the bladders 440 so that the entire gastric band assembly is in fluid communication.

Referring to FIG. 86, a graph illustrates the swallowing simulation in which the band only, the band plus bladders, and the band plus bladders plus restrictor are plotted. As food reaches the gastric band 432 and the stoma 436, pressure inside the stoma area proximal to the gastric band starts to increase due to esophageal motility. This causes the pressure inside the gastric band (intra-band pressure) to increase rapidly to create a high pressure wave. As used herein, a high pressure wave is an intra-band pressure wave that is caused by the patient swallowing. Referring to FIG. 86, the increase starts at around 30 mmHg and continues to build up to around 65 mmHg. Once the intra-band pressure inside the band exceeds the fluid pressure inside the bladders 440, fluid starts to flow out of the balloon 434 and into the bladders 440. In doing so, the fluid pushes the ball 408 against the ball seat 410 and effectively blocks the main flow channel 406 so that fluid does not flow through the main flow channel from the balloon to the bladders. Fluid can still flow through the bypass channel 420, albeit at a much reduced rate. This outflow of fluid from the balloon 434 to the bladders 440 continues until the pressure of the gastric band equals the pressure in the bladders 440. Again referring to FIG. 86, the equalized pressure is again around 30 mmHg. Once the intra-band pressure in balloon 434 falls below the pressure of the bladders 440, the fluid will reverse and flow from the bladders 440 to the balloon 434 and thereby disengage the ball 408 from the ball seat 410 so that fluid flows through the main channel 406 from the bladders to the balloon. The fluid rushes back to the balloon 434 at a very high rate since the cross-sectional area of the main flow channel is much greater than the cross-sectional area of the bypass channel. This effect is shown in the pressure wave plot of FIG. 86 where the slope of the pressure increase is flatter than that of the pressure decrease indicating that the flow leaves the bladders more quickly than it enters the bladders. This is very important because the period which the intra-band pressure is lower than the bladder pressure is much shorter than the period which the intra-band pressure is higher than the bladder pressure. Thus, in order to achieve zero net flow (or minimize net flow) of fluid from the band to the bladders during each pressure wave, the return flow rate from the bladders to the balloon has to be higher than the outflow rate in the opposite direction.

Again referring to FIG. 86, with the band only in the gastric band assembly, the patient will experience pressure spikes when swallowing food or liquids that is believed to give the patient a feeling of being satiated and thereby promoting the desired weight loss. With the band and bladders only in the gastric band assembly, the pressure wave shows that fluid flows from the band to the bladders and back at a rapid rate, so that there is less of a pressure spike with the bladders in the system. With just the gastric band and bladders in the system, the patient may not gain that sense of being satiated when swallowing food and thus reduce the effectiveness of the gastric band assembly in promoting weight loss. With the gastric band, bladders and flow restrictor 400 in the gastric band assembly 430, the pressure wave as shown in FIG. 86 mimics the pressure wave developed by the gastric band only. Thus, by incorporating the flow restrictor 400, the pressure spike is substantially preserved thereby promoting the patient feeling satiated while swallowing and further promoting the desired weight loss.

As previously disclosed, and as shown in FIGS. 79-82 for example, a non-biased ball 408 is positioned adjacent the main flow channel 406 and will block the main flow channel when seated on ball seat 410. The non-biased ball 408 is designed to be highly responsive to fluid flow and to act very quickly in response to changes in fluid flow rate and the direction of fluid flow. For example, the non-biased ball 408 will move toward and seat on ball seat 410 with fluid flow rates as low as a range from 0.5 mL per minute to about 2.0 mL per minute, and remain firmly seated thereby blocking the main flow channel. Similarly, when the pressure gradient reverses, the fluid flow will reverse and unseat the non-biased ball so that fluid can resume flow through the main flow channel. Again, a non-biased ball is highly responsive so that a reverse flow range of about 0.5 mL per minute or less to about 2.0 mL per minute is sufficient flow rate to unseat the ball and keep it unseated until the pressure gradient changes direction again.

One important feature of the flow restrictor 400 is the capability of the bypass channel 420 to permit the balloon 434 to be emptied of fluid in a quick and controlled manner. For example, if the patient is experiencing extreme tightness in the gastric band, the physician may have to temporarily remove all of the fluid in the balloon, thereby allowing the size of the stoma to increase and provide relief for the patient. The fluid removal is accomplished by inserting a standard syringe needle into the refill port 444 and withdrawing fluid in a known manner. In a gastric band assembly without a flow restrictor, the fluid removal rate from the band is about seven mL per ten seconds, and with the flow resistor in place the fluid removal rate is about two mL per ten seconds (with a bypass channel having a 0.006 inch by 0.006 inch cross-sectional area). This fluid removal rate will drain the band in about two minutes. Different fluid removal rates are contemplated by using flow restrictors with bypass channels having different cross-sectional areas than indicated. Thus, the flow removal rate could range from 0.5 mL per ten seconds up to 4 mL per ten seconds, and still be acceptable clinically.

The foregoing disclosure regarding a flow restrictor incorporated into an LABG having a bladder system is important to the time-dependent aspects of swallowing, referred to herein as “progressive distensibility.” In principle, the analyses presented herein solved the following set of time-dependent differential equations:

$$\frac{d {V}_{LAGB}}{d t} = {}_{+}^{}{Q}{}_{cc}^{} ( {P}_{bladder} - {P}_{LAGB} )$$

$$\frac{d {V}_{bladder}}{d t} = {}_{+}^{}{Q}{}_{cc}^{} ( {P}_{bladder} - {P}_{LAGB} )$$

where V_{LAGB} and V_{bladder} represent the internal fill volumes of the LAGB and bladder components, respectively; and Q_{CC}(P_{bladder}−P_{LAGB}) represents the pressure-head- and directionally-dependent flow magnitude across the flow restrictor, with P_{bladder} and P_{LAGB} representing the internal pressures within the bladder and LAGB components, respectively, and P_{bladder}−P_{LAGB} representing the effective pressure head across the flow restrictor (with positive and negative difference values associated with “forward” and “reverse” flows, respectively).

Experimentally-derived in-silico mathematical models of LAGB pressure-volume-diameter relationships and bladder system pressure-volume relationships were referenced while solving of these equations.

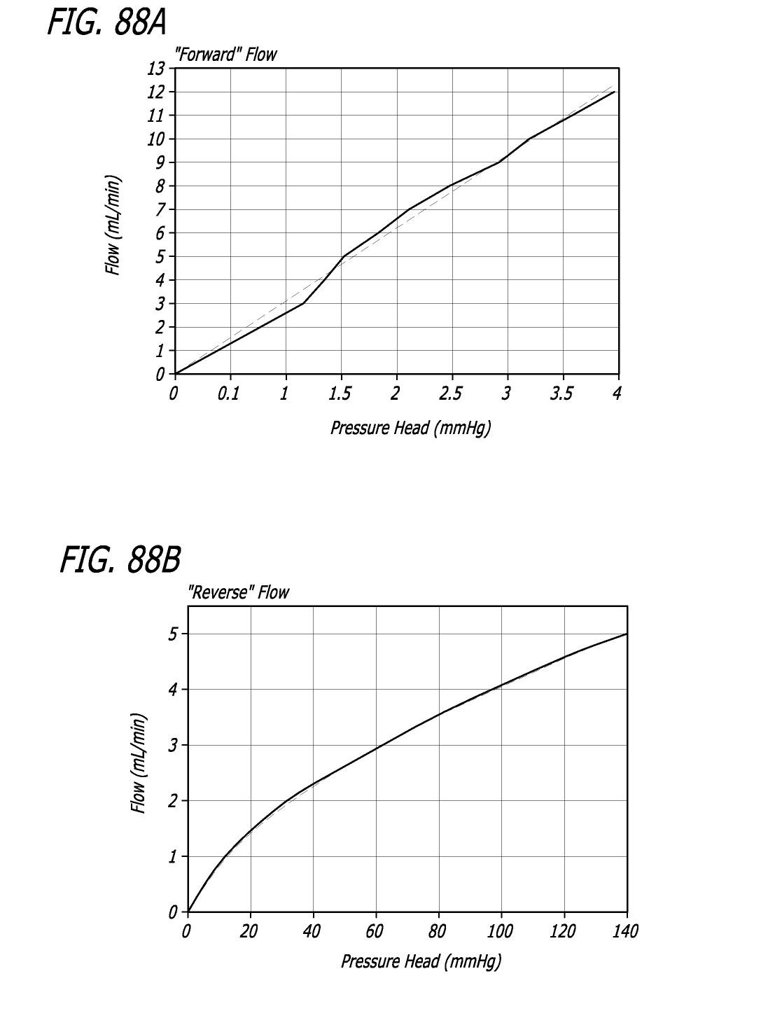

Experimentally-derived models of flow restrictor pressure-flow relationships were also utilized in these analyses. A feature of particular interest herein is the asymmetric flow characteristics of the flow restrictor. As illustrated graphically in FIGS. 88A and 88B (data points from bench-based in-vitro experimental measurements), the flow through the flow restrictor is asymmetrically dependent on the applied pressure head across the flow restrictor, with the “reverse” flow substantially restricted for a given absolute pressure head as compared to the “forward” flow (note the significant differences in x- and y-axes). The overlaid dotted lines plot the least squared-regressive fits associated with these data—linear fit for “forward” flow, and square-root fit for “reverse” flow. These derived flow vs. pressure head equations were used in the in-silico differential equation mathematical model described supra.

The intent of these analyses was to quantitatively estimate the induced distension of the band stoma during primary and/or secondary swallow transients (e.g., as measured as the change in band stoma diameter, etc) with the LAGB alone or with the LAGB connected to a bladder system via a flow restrictor. A further intent was to determine how these induced distensions were affected by the flow restrictor flow magnitudes and flow ratios.

A series of time-dependent simulations (based on the model equations described supra) were performed using the following input conditions:

| LAGB Type | Ethicon SAGB-VC or Allergan APS |
|---|---|
| Baseline Band Stoma Diameter | 21 mm |
| Baseline Stoma Contact Pressure | 10 mmHg |
| Swallow Peristalses (Transients) | |
| Transient Shape | Triangular |
| Transient Peak Amplitude | 30 mmHg |
| Transient Duration (Period) | 10 sec |
| Transient Count | 5 |

These swallow peristalses were assumed to act on the LAGB via direct superposition onto the LAGB stoma contact pressure. Thus, for these simulations, the stoma contact pressure followed a triangular pattern with a baseline of 10 mmHg and peak amplitude of 40 mmHg (i.e., 10+30).

Different input conditions were also explored but did not qualitatively change the fundamental conclusions described infra.

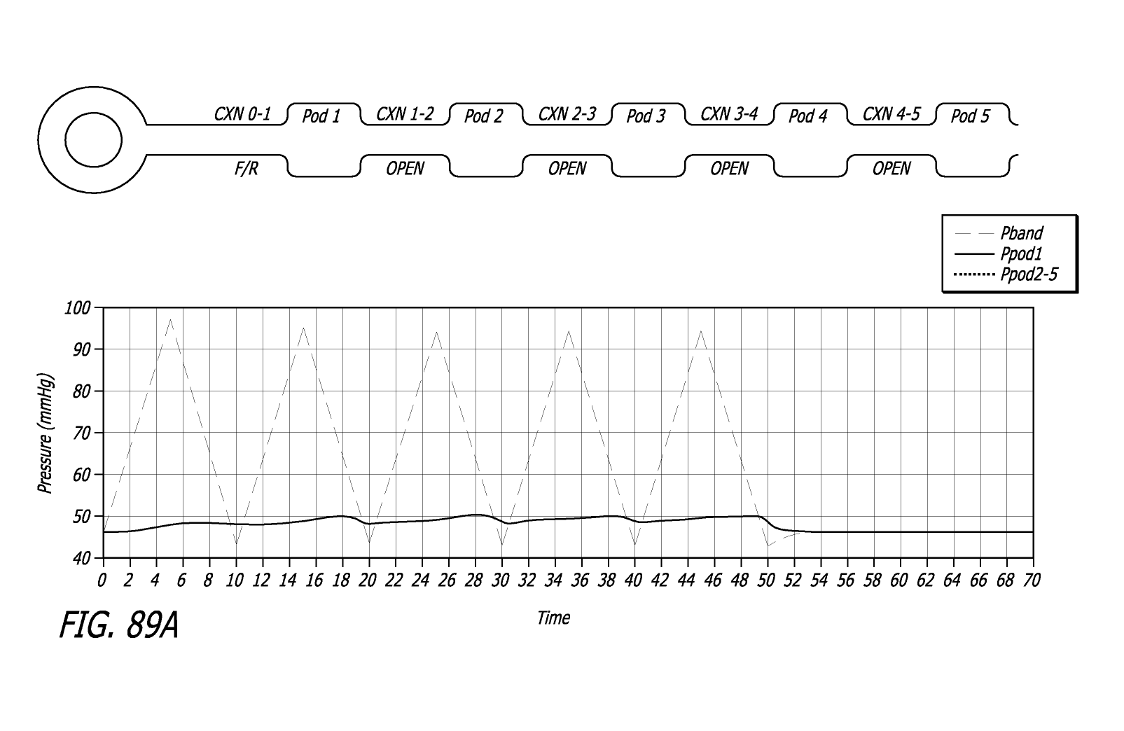

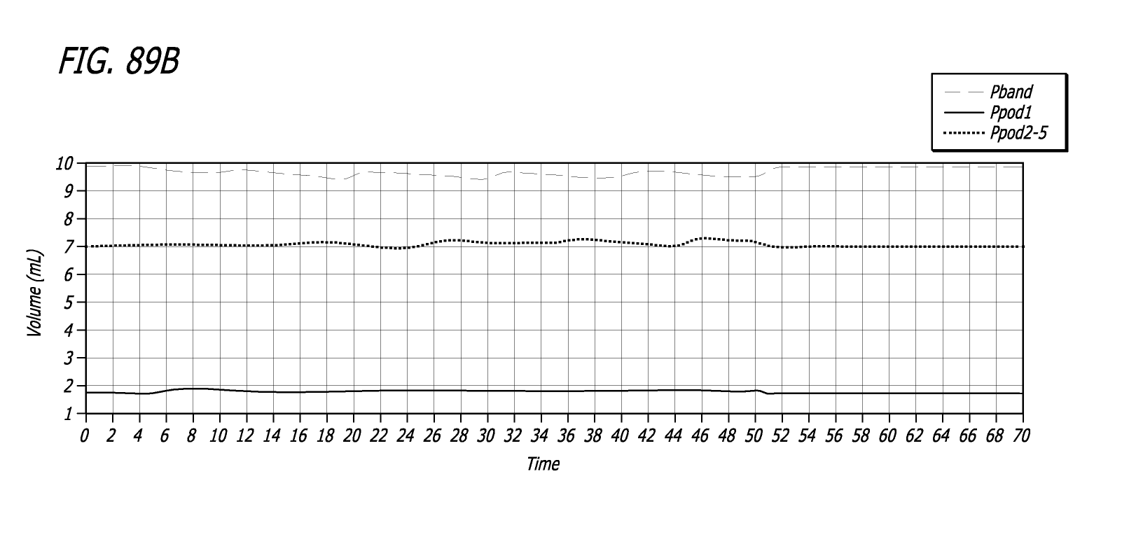

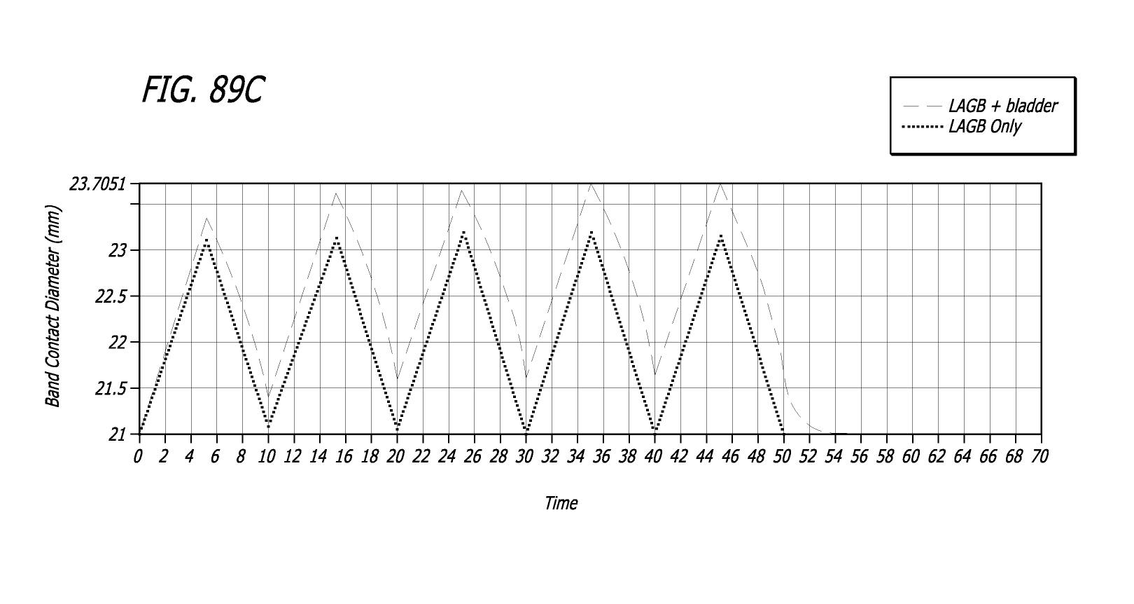

FIGS. 89A-89C present a screen capture of the results from a representative simulation. It displays three sets of temporal plots as generated based on the associated defined input conditions (left input values) and system configuration (top schematic): LAGB-plus-bladder component-level Internal Pressures vs. Time (FIG. 89A), LAGB-plus-bladder component level Fill Volumes vs. Time (FIG. 88B), and LAGB Contact Diameter vs. Time (FIG. 89C). FIG. 89C also includes the band contact diameter vs. time plot of the equivalent LAGB-only configuration (for easy comparison). Note that with each swallow peristalsis, the LAGB contact diameter increases in concert with the applied addition contact pressure. However, whereas the band contact diameter attains the same peak diameter across all five peristalses for the LAGB-only configuration (ΔSD=2.12 mm), the band contact diameter attains progressively larger peak diameters for the LAGB-plus-bladder configuration (ΔSD=2.34→2.71 mm).

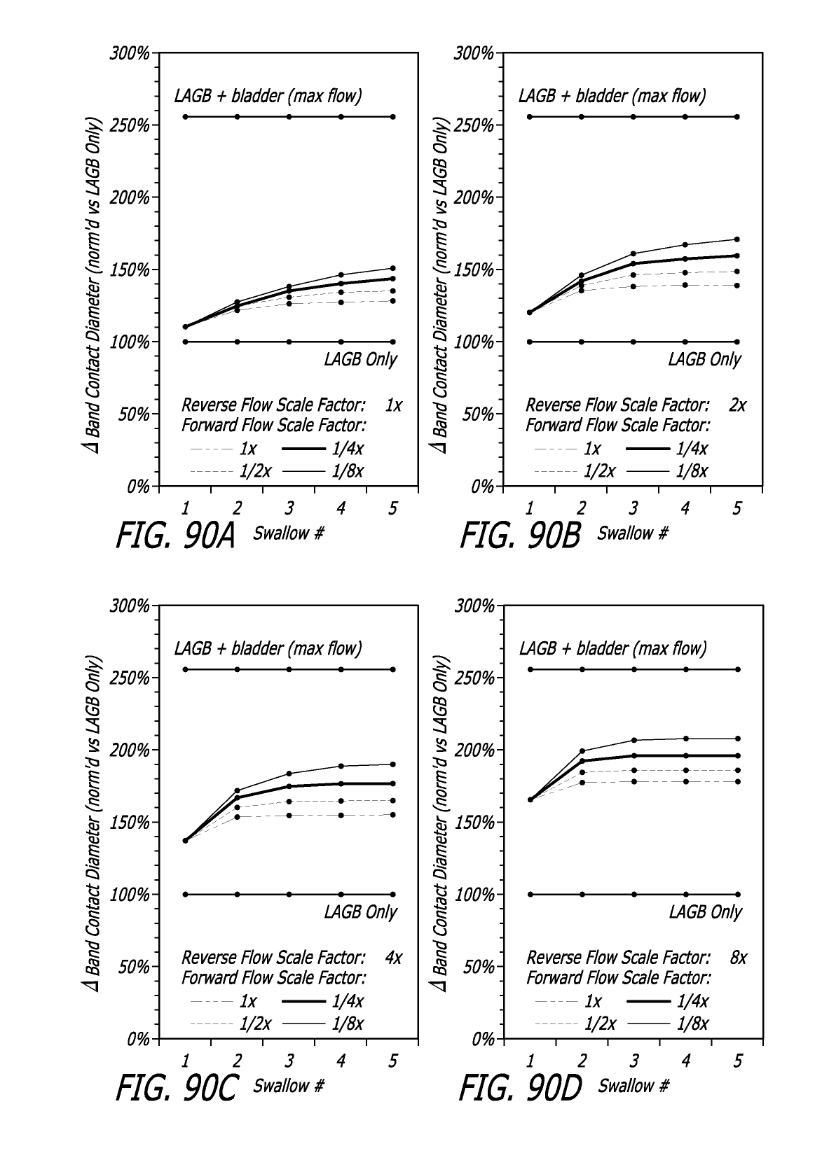

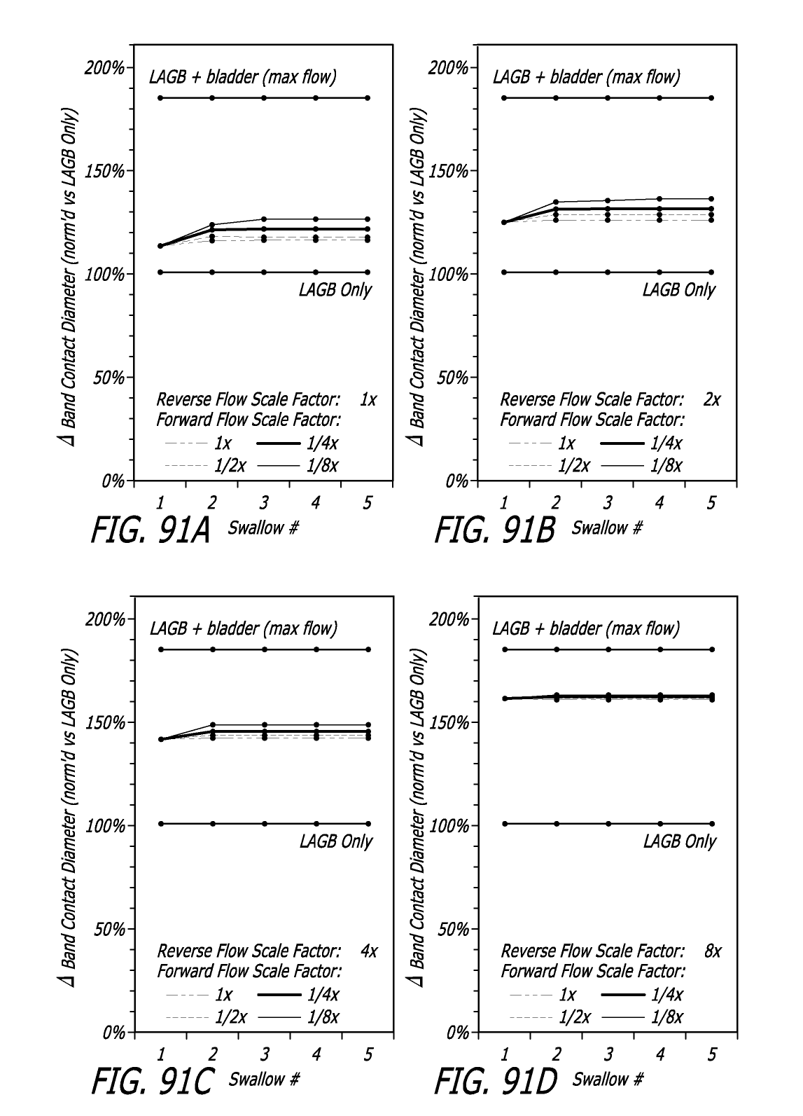

Furthermore, the magnitude and course of this progressive distensibility can be modulated via modifications of the absolute and relative flows (and flow ratios) through the flow restrictor. This simulation was repeated multiple times for a range of relative “forward” and/or “reverse” flow rates through the flow restrictor (implemented by applying associated “flow scale factors” to the forward and reverse flow relationships described in FIGS. 88A and 88B). A “max flow” restrictor condition was also simulated to identify the upper limit of distensibility. The results of these simulations are summarized in FIGS. 90A-90D (for the SAGB-VC LAGB) and FIGS. 91A-91D (for the APS LAGB). These sets of plots graph the progression of peak change in LAGB contact diameter (normalized by the associated LAGB-only configuration) with each swallow peristalsis (labeled on the plots as Swallow #) for different combinations of “forward” and “reverse” flow scale factors.

While the example and results described above utilized a flow restrictor having asymmetric flow characteristics, such asymmetry is not a necessary requirement to achieve progressive distensibility. Progressive distensibility can also readily be achieved with the use of flow restrictors having symmetric (i.e., equivalent) “forward” and “reverse” flow characteristics.

Thus, the addition of the bladder system—and, in particular, in conjunction with the flow restrictor—provides progressive distensibility to the LAGB stoma in the event the food bolus is not successfully cleared during the primary swallow peristalsis. Advantageously, this progressive distensibility feature may progressively improve the possibility/ability to successfully clear the food bolus during each secondary swallow peristalsis.

The use of an asymmetric-flow restrictor positioned between an LAGB and a bladder system provides increased and progressive distensibility to the LAGB stoma as described supra. Subsequent in-silico and in-vitro experimentation has demonstrated that such enhanced distensibility performance is completely feasible through the use of an intervening restrictive member having symmetric flow restriction behaviors as well.

A series of in-silico simulations was performed to investigate the pouch-stoma-LAGB interactions during swallowing, both as a LAGB-only configuration and as a LAGB plus bladder configuration with a symmetric flow restrictor interposed therewith in which the conductance of the symmetric flow restrictor connection ranged from zero (i.e., equivalent to LAGB-only) to “infinity” (i.e., max flow, such that there was never any pressure differential between LAGB and bladder components). Both asymmetric and symmetric conductance profiles were investigated, although only the results from the symmetric conductance profiles are specifically summarized here.

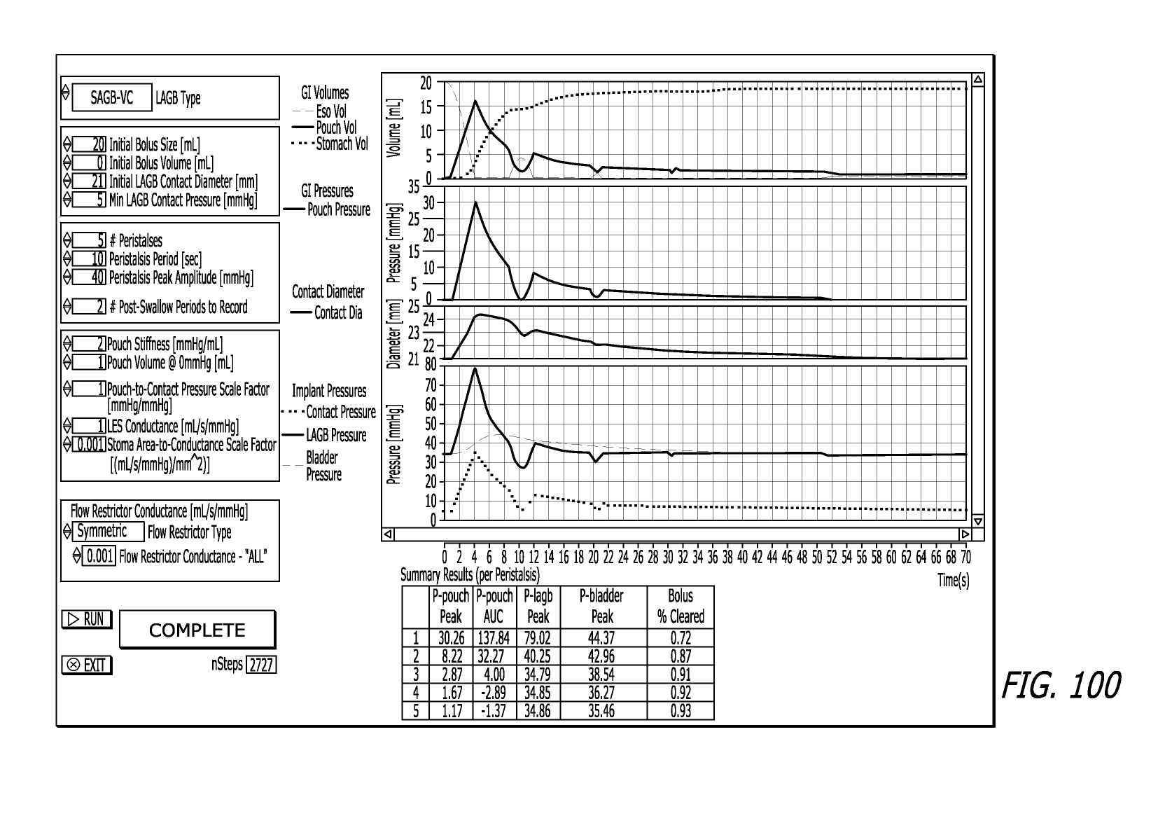

FIG. 100 presents a screen-capture of the results from a representative simulation. These results illustrate the time-courses of various parameters (e.g., volumes, pressures, etc) as multiple simulated peristalses attempt to transport a food bolus from the esophagus, into the pouch, through the LAGB-created stoma, and finally into the stomach. In this illustrated simulation, the flow restrictor conductance is symmetric at 0.01 mL/s/mmHg (equivalent to a symmetric flow restrictor supporting a 5 mmHg backpressure at a flow rate of 3 mL/min). Under these conditions, the increased distensibility (compared to LAGB-only [not shown]) enabled the primary peristalsis to pass ˜72% of the bolus volume. In contrast, an LAGB-only configuration under the same simulated conditions only passes ˜52% of the bolus volume during the primary peristalsis.

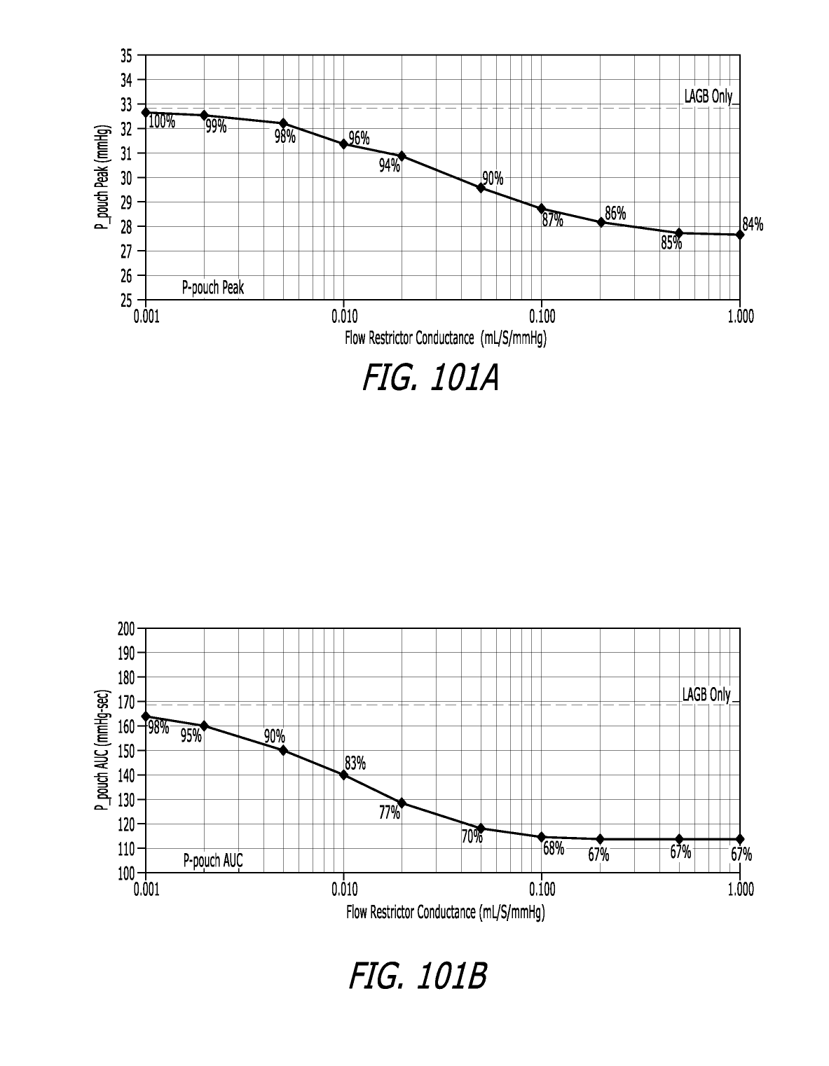

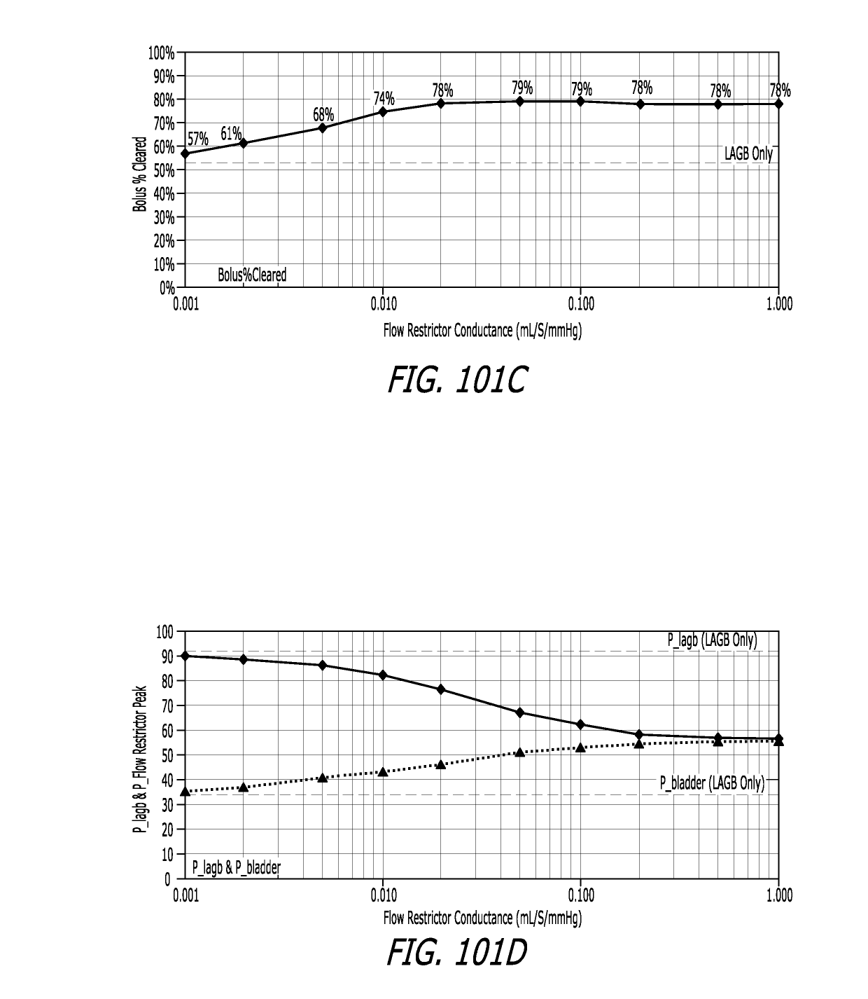

This simulation was repeated across a broad range of symmetric flow restrictor conductances, and the subsequent results associated with the first/primary peristalsis were summarized and plotted (see FIGS. 101A-101D for an SAGB-VC). In general, these results illustrate the trade-off between pouch pressure metrics (e.g., peak, area-under-the-curve [AUC]) versus % bolus volume cleared. “AUC” refers to area under the pressure v. time curve in mmHg*sec.

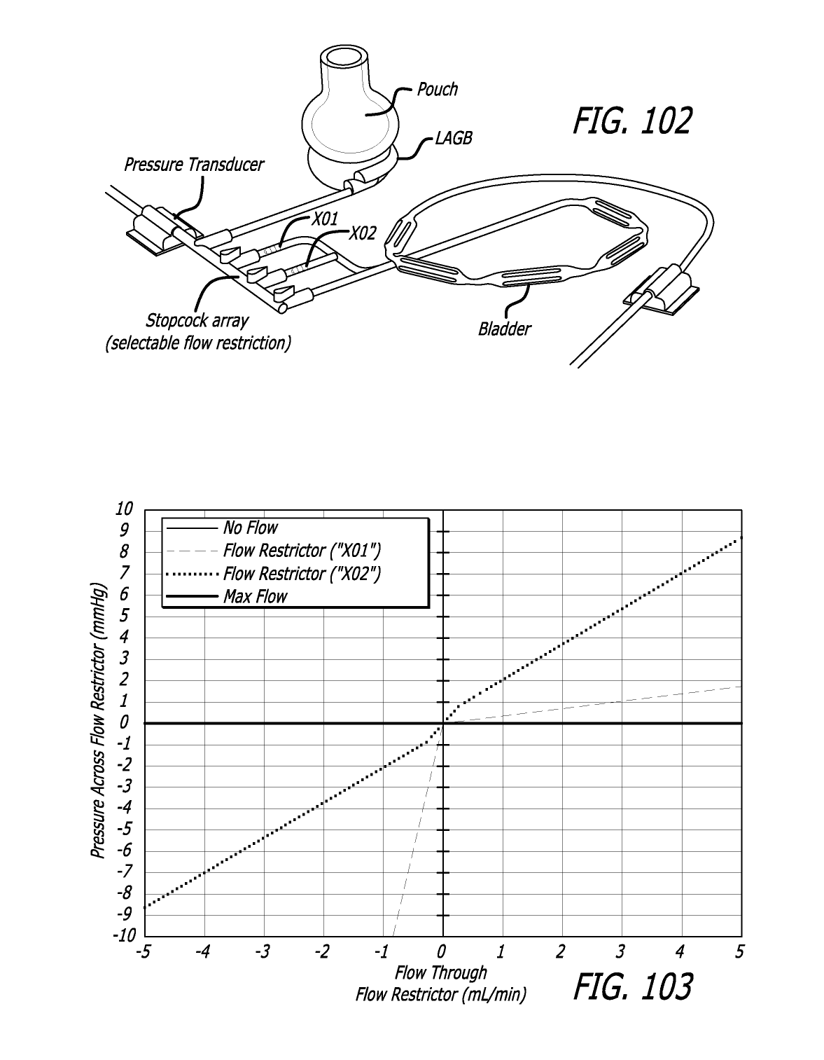

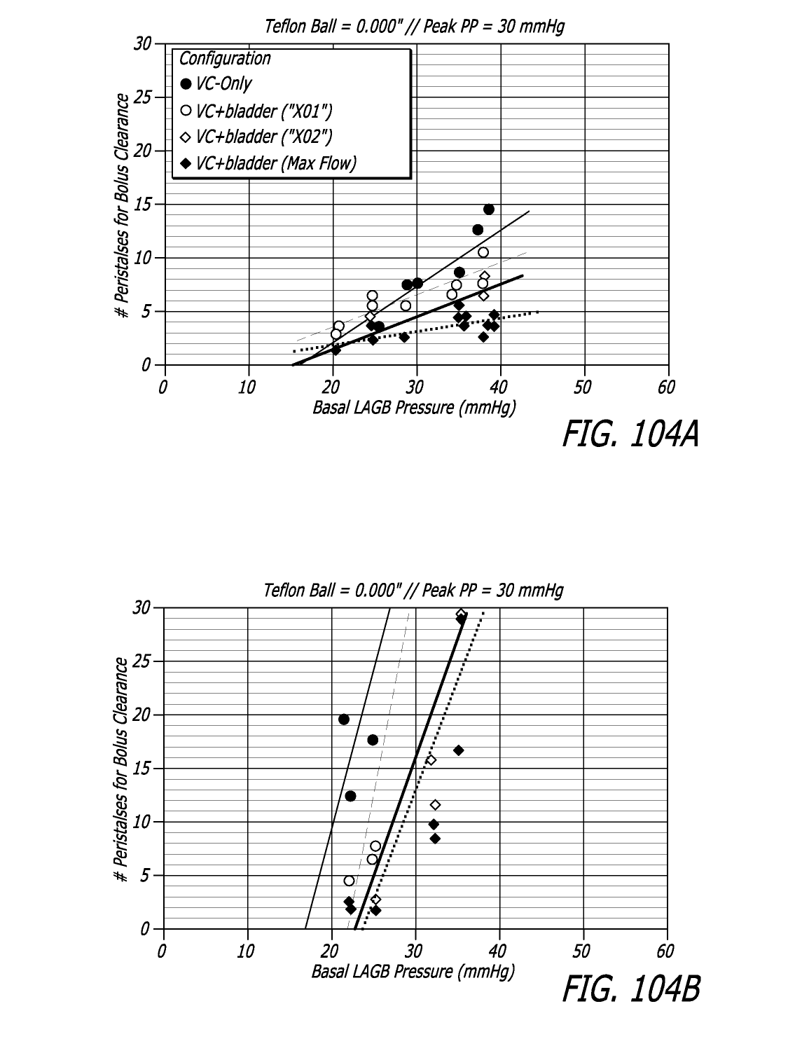

A series of bench experiments was conducted to investigate how changes in symmetric flow restrictor conductance in a LAGB plus bladder configuration would impact rates of bolus clearance. In this set-up as shown in FIG. 102, the LAGB was placed around simulated stomach tissue (obtained from SynDaver), thereby establishing a pouch and stoma. A bladder system as disclosed herein was connected to the LAGB via a selectable set of flow connectors having varying conductance profiles: (a) zero conductance (i.e., LAGB-only); (b) an asymmetric flow restrictor (labeled “X01” which is a prototype asymmetric flow restrictor made by CAVU Medical, Inc., Menlo Park, Calif.); (c) max conductance (i.e., max flow condition); and (d) a prototype symmetric flow restrictor (labeled “X02” also by CAVU Medical, Inc.) having a symmetric conductance of ˜0.01 mL/s/mmHg (equivalent to a connector supporting a 5 mmHg backpressure at a flow rate of 3 mL/min). FIG. 103 illustrates the conductance profiles of these connectors and flow restrictors, and notably how the conductance profile of the prototype restrictor “X01” is asymmetric through zero while the prototype connector “X02” is symmetric.

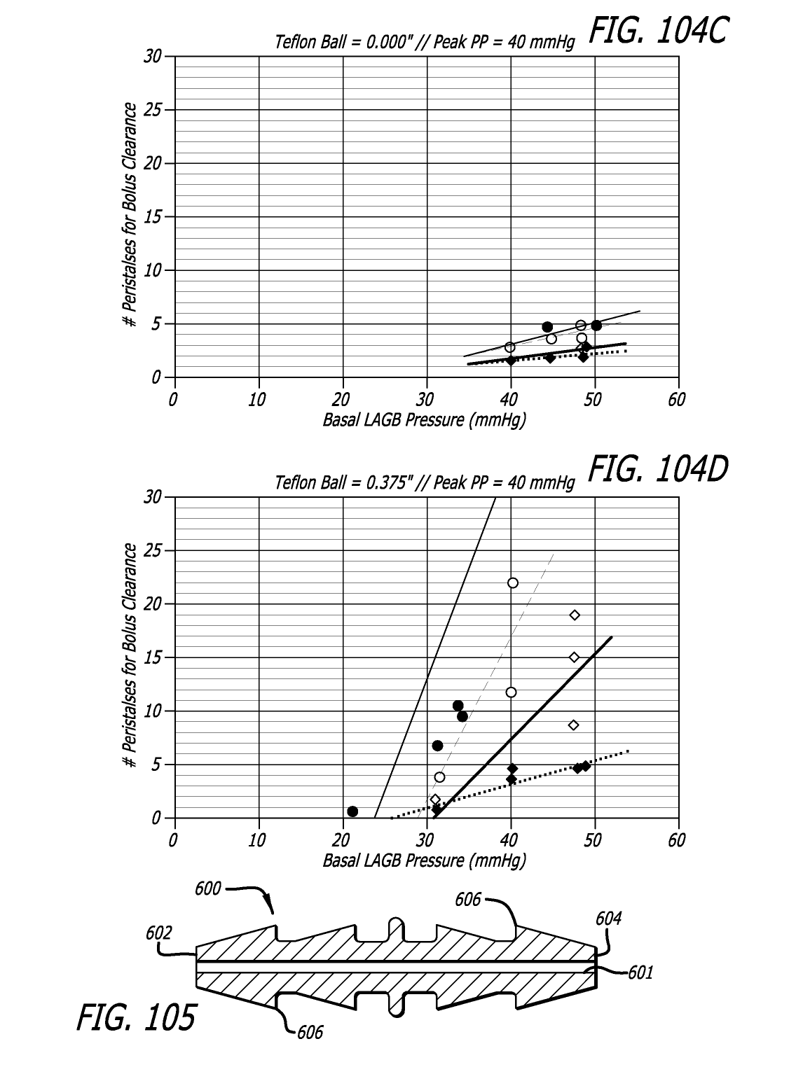

In these experiments, the LAGB (with or without an attached flow restrictor system) was filled to a specified basal intra-band pressure, thereby creating a stoma with an associated dimension (e.g., higher basal intra-band pressures resulted in narrower/tighter stomas). A standardized 20 mL bolus mash with or without an obstructive solid sphere was then placed into the pouch above the LAGB-formed stoma. Then the pressure within the pouch was cyclically varied between zero and a specified peak pressure (Peak PP) at a defined period (nominally 10 seconds). Pressures within the pouch, the LAGB, and the bladders were simultaneously recorded during these tests. The number of cycles required to clear the standardized bolus through the stoma was also determined. These latter results were then plotted as a function of LAGB basal pressure. The SAGB-VC test results are summarized in FIGS. 104A-104D. Of note, the “X02” symmetric flow restrictor results demonstrate how, at least under these experimental conditions, the increased and progressive distensibility it provides translates into a significantly improved ability to transit these boluses as compared to the LAGB-Only and even the LAGB plus bladder configurations with the “X01” asymmetric flow restrictor. It is believed that all flow restrictors disclosed herein having a conductance>0 would result in an “increased” distensibility relative to the LAGB-only configuration while any/all flow restrictors having 0<conductance<infinity would result in “progressive” distensibility.

A flow-restrictive connection between an LAGB and a bladder system that provides the enhanced distensibility behavior described above can be achieved through various means. For example, a discrete connector similar to the prototype restrictor “X01” could be utilized to interconnect an LAGB and a bladder system, but the internal geometry of the “X01” restrictor provides for symmetric restricted flow. For example, as shown in FIG. 105, a symmetric flow restrictor 600 having a length of about 0.6 inch with a through lumen 601 having an internal through diameter of ˜0.019 inch provides a symmetric conductance of ˜0.01 mL/s/mmHg. Of course, smaller or larger conductances could be established with an appropriate modification to this internal diameter and/or length. In the embodiment shown in FIG. 105, the symmetric flow restrictor 600 has a distal end 602 and a proximal end 604 that are configured for secure attachment to tubing that is connected to the LAGB balloon and the bladder in a manner similar to that shown in FIGS. 79-85 for asymmetric flow restrictor 400. The tubing slides over ridges 606 on the outer surface of the symmetric flow restrictor 600 and is firmly attached since the ridges have sharp edges to engage the inside wall of the tubing.

Alternatively, the tubing extending between an LAGB balloon and bladder could be designed with a narrow internal diameter so that the flow through that tubing section is restricted to the desired effective conductance. For example, a 4-inch tubing segment with an internal through diameter of 0.037 inch should provide a symmetric conductance of ˜0.01 mL/s/mmHg. Of course, smaller or larger conductances could be established with an appropriate modification to this internal diameter and/or length.

### LAGB Pressure-Volume-Diameter Analysis

A series of in-vitro bench experiments was conducted to evaluate the pressure-volume-diameter characteristics of LAGB's (particularly Allergan Lap-Band AP Standard and Ethicon SAGB VC). The set-up consisted of the band portion of the selected LAGB secured around a modified EndoFLIP impedance planimetry balloon (Product Ref EF-325; Crospon, Inc.; with a 35-mm diameter replacement balloon). For each targeted step in LAGB total fill volume, the EndoFLIP balloon (the “stomach”) was first initialized with sufficient volume to establish a maximal band-to-stomach contact pressure (generally 50-60 mmHg), and then the EndoFLIP balloon was slowly evacuated via a syringe pump until the measured contact pressure dropped below 5 mmHg. Intra-band pressure (BP), band-to-stomach contact diameter (SD), and band-to-stomach contact pressure (SP) were all simultaneously acquired/recorded during each fixed-volume run (SD via the EndoFLIP system; BP and SP via an HP Pressure Monitor with M1006A modules; all acquired using a National Instruments USB-6009 DAQ hardware and a custom LabVIEW program).

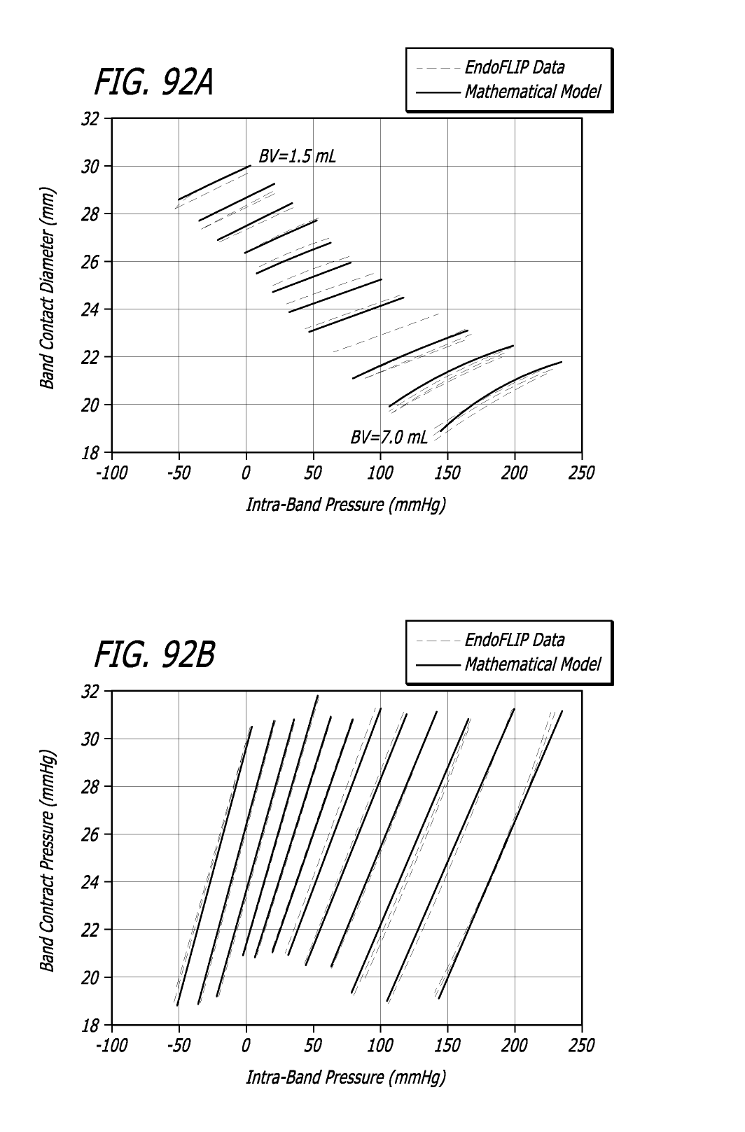

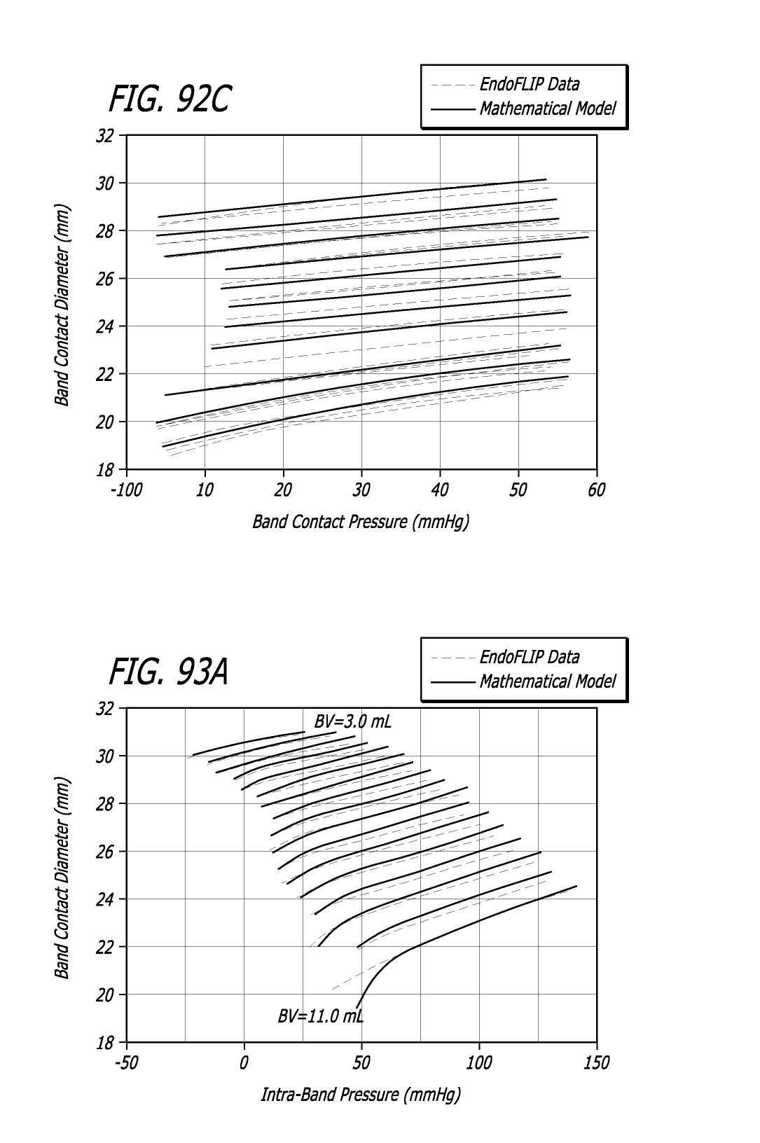

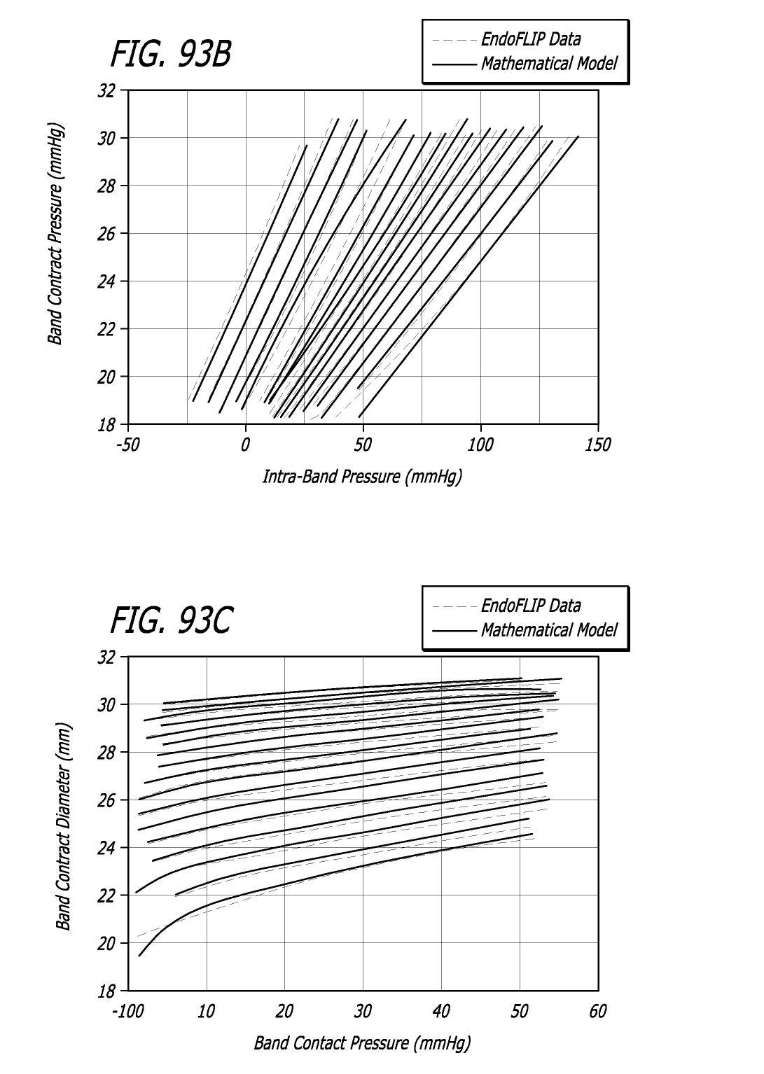

These EndoFLIP data were subsequently analyzed, and a mathematical model was constructed to simulate these pressure-volume-diameter relationships. The acquired EndoFLIP data and its model-equivalent curves are disclosed in FIGS. 92A-92C (Allergan Lap-Band AP Standard) and 93A-93C (Ethicon SAGB VC).

### Bladder Pressure-Volume Analysis

A series of in-vitro bench experiments was conducted to evaluate the pressure-volume characteristics of bladders as disclosed herein having model numbers C10-A and C10-E. The set-up consisted of the selected bladder connected to a syringe pump. The bladder was first primed with saline (to eliminate any air bubbles) and then fully evacuated of that saline such that the internal pressure was <−300 mmHg. Saline was then slowly infused via the syringe pump at a known constant rate, and the resultant internal pressures was acquired/recorded (via an HP Pressure Monitor with an M1006A module; acquired using a National Instruments USB-6009 DAQ hardware and a custom LabVIEW program).

It is believed that the addition of a bladder to an LAGB better preserves stoma size over time than an LAGB only. The stomach tissue encompassed by an LAGB can be generally described by an outer dimension (SDo) (e.g., diameter, area, etc.) and an inner dimension (SDi) (both>=0, with SDo>SDi). Hence, if these dimensions are assumed to be diameters, the thickness of the stomach can be described as: ST=(SDo−SDi)/2. The stomach inner (stoma) dimension has an “unstrained” lumen size (SDi0) that can be forced smaller as some function of applied net contact pressure: e.g., SDi=SDi0−F(P−P0). The stomach tissue encompassed by LAGB remodels (i.e., wall thickness decreases) at a rate vs. time proportional to an applied net contact pressure: e.g., dST/dt=−K*(P−P0). While this equation is a very simple 1st-order linear model, certainly other higher-order and/or nonlinear models are possible. For a fixed LAGB fill volume, the applied contact pressure decreases as the stomach tissue encompassed by the LAGB remodels (e.g., the stomach's outer diameter decreases): e.g., P=G(SDo).

The addition of a bladder to an LAGB effectively changes the behavior of function “G” above (i.e., it becomes less steep), as illustrated, for example, in FIG. 65. Since everything else is the same, this framework supports the premise that SDi will be better preserved over time with a bladder added to an LAGB versus the LAGB alone.

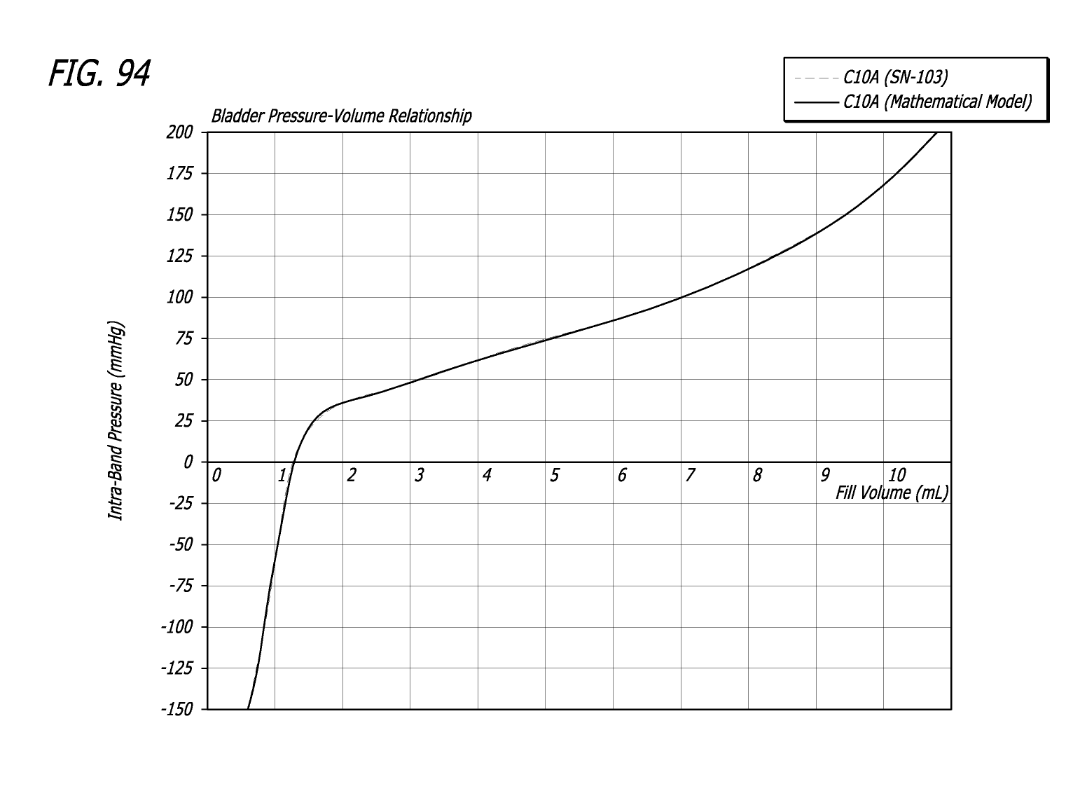

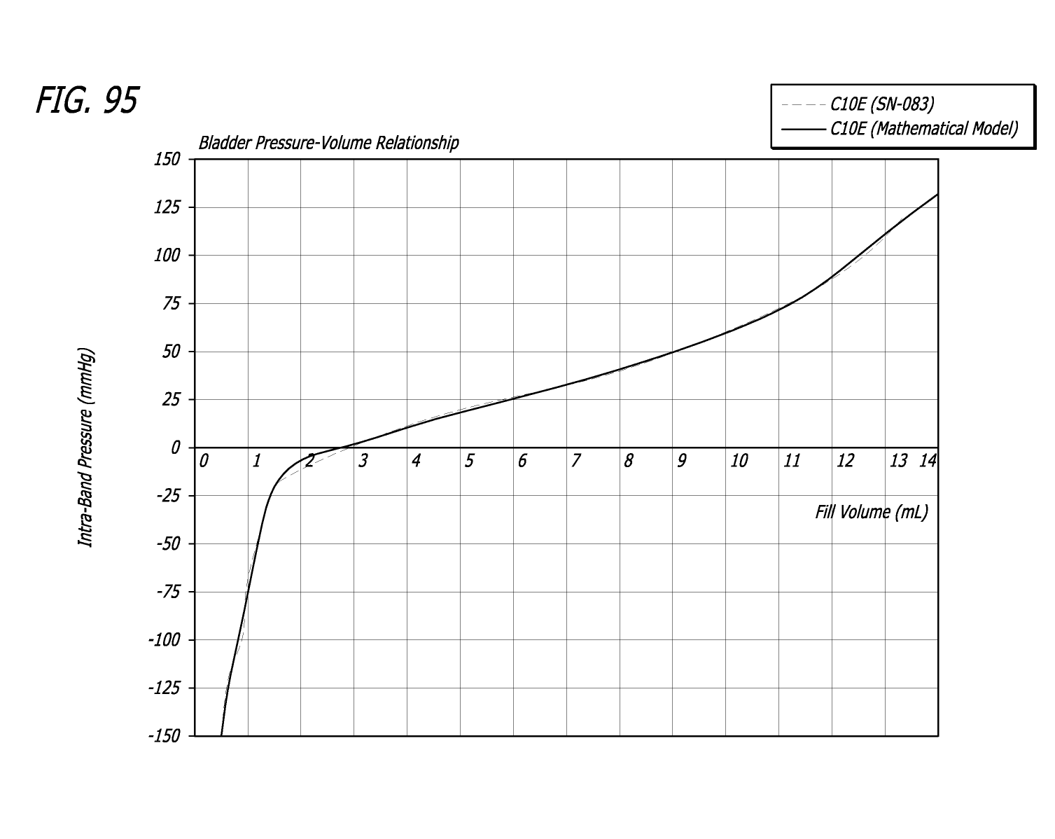

These bladder PV data were subsequently analyzed, and a mathematical model was constructed to simulate these pressure-volume relationships. The acquired bladder PV data and its model-equivalent curves are disclosed in FIGS. 94 (bladder model C10-A) and 95 (bladder model C10-E). The bladders models C 10-A and C10-E are available from CAVU Medical, Menlo Park, Calif.

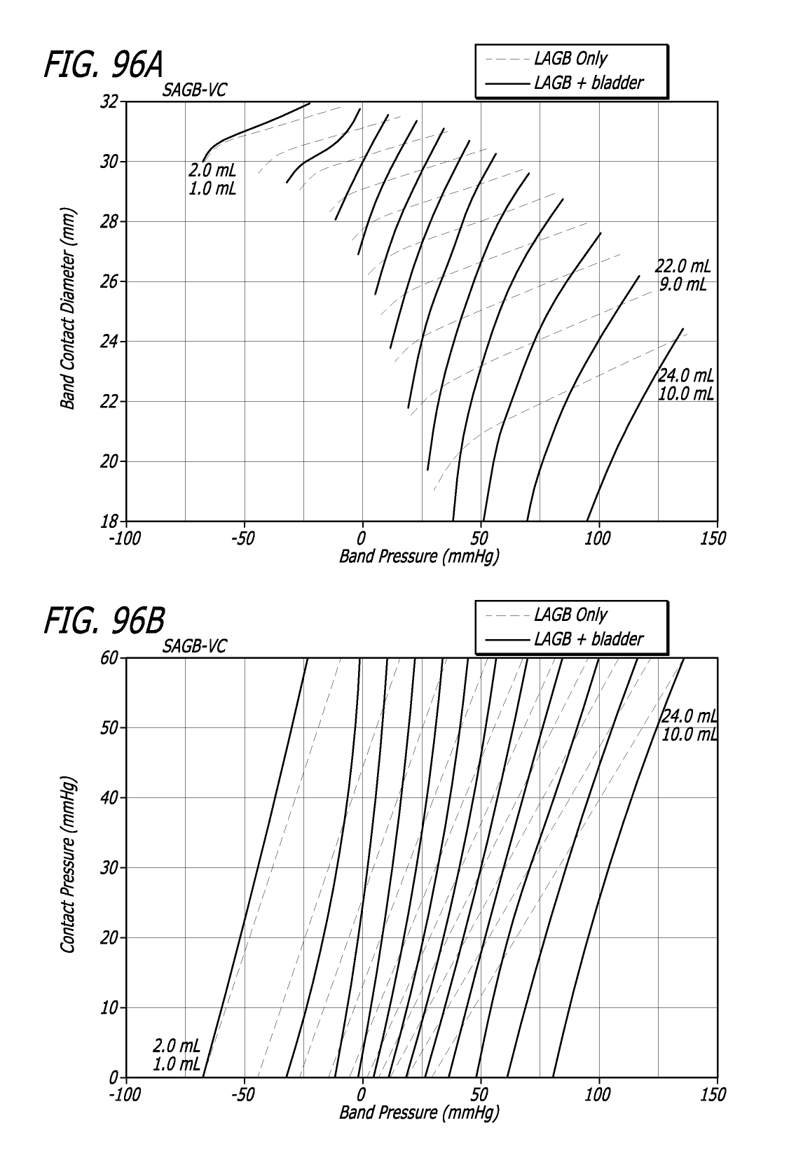

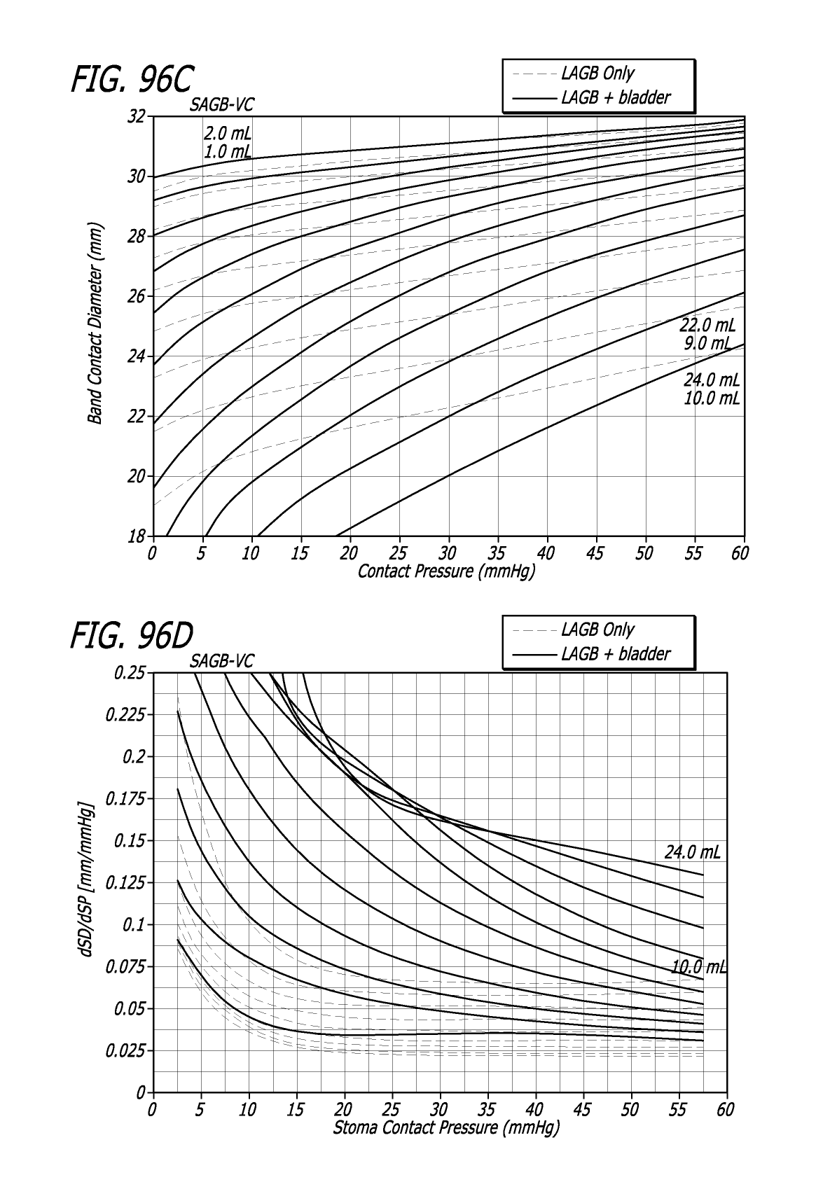

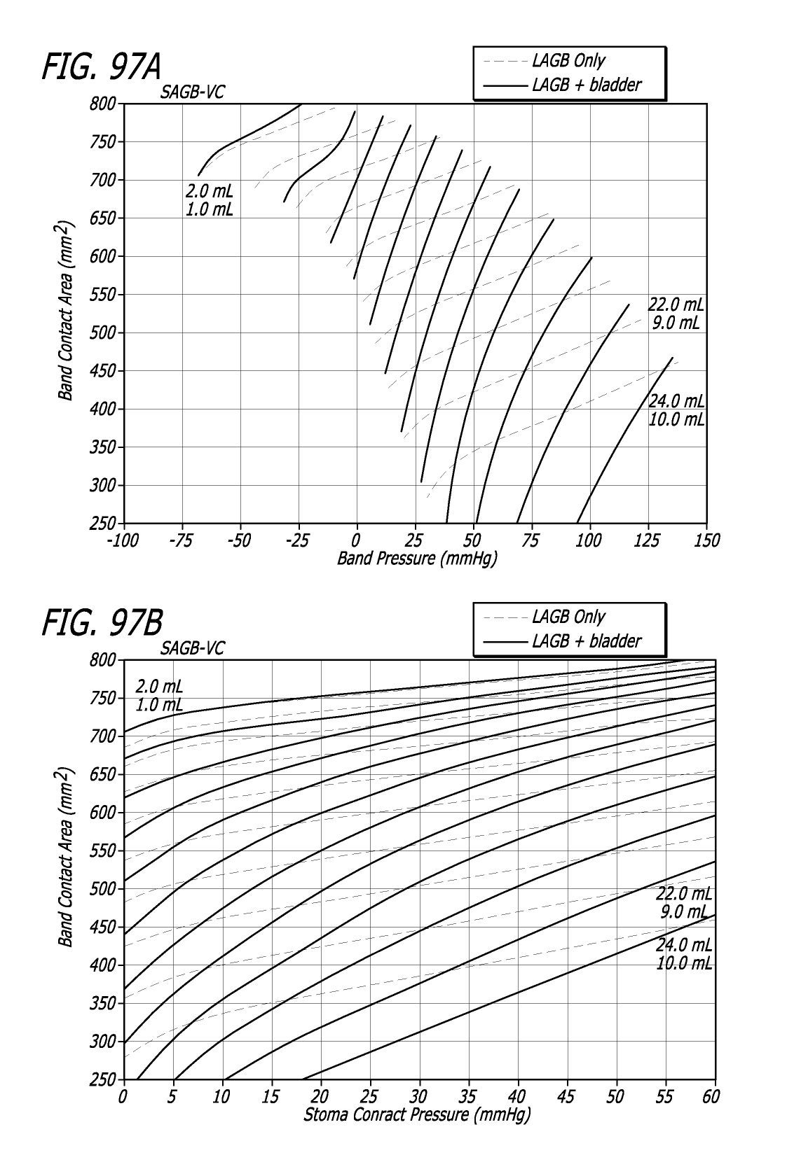

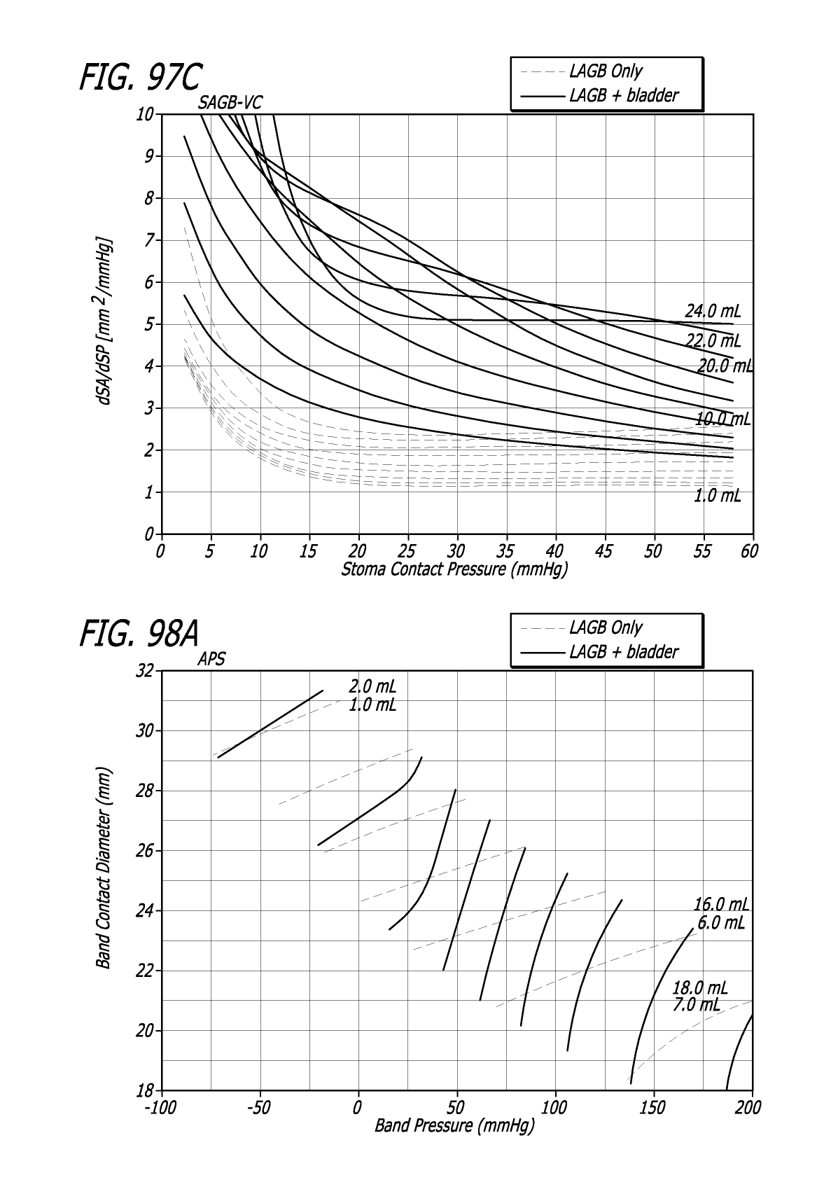

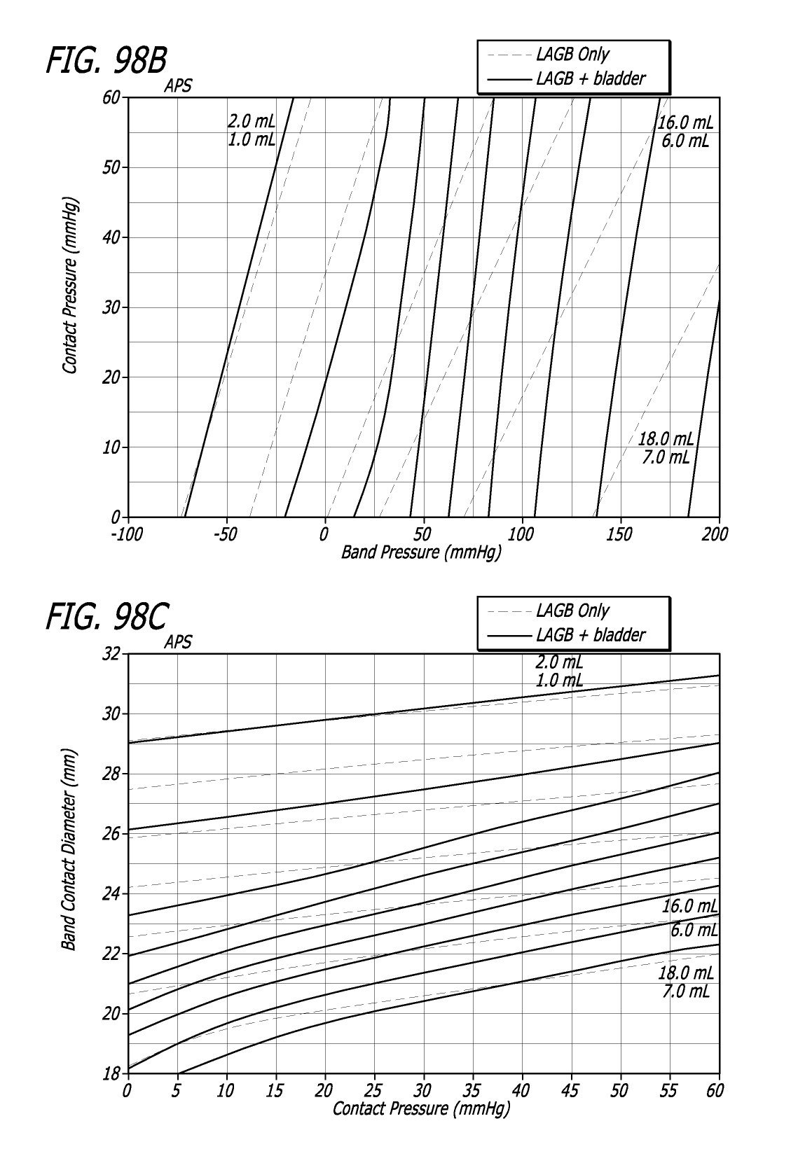

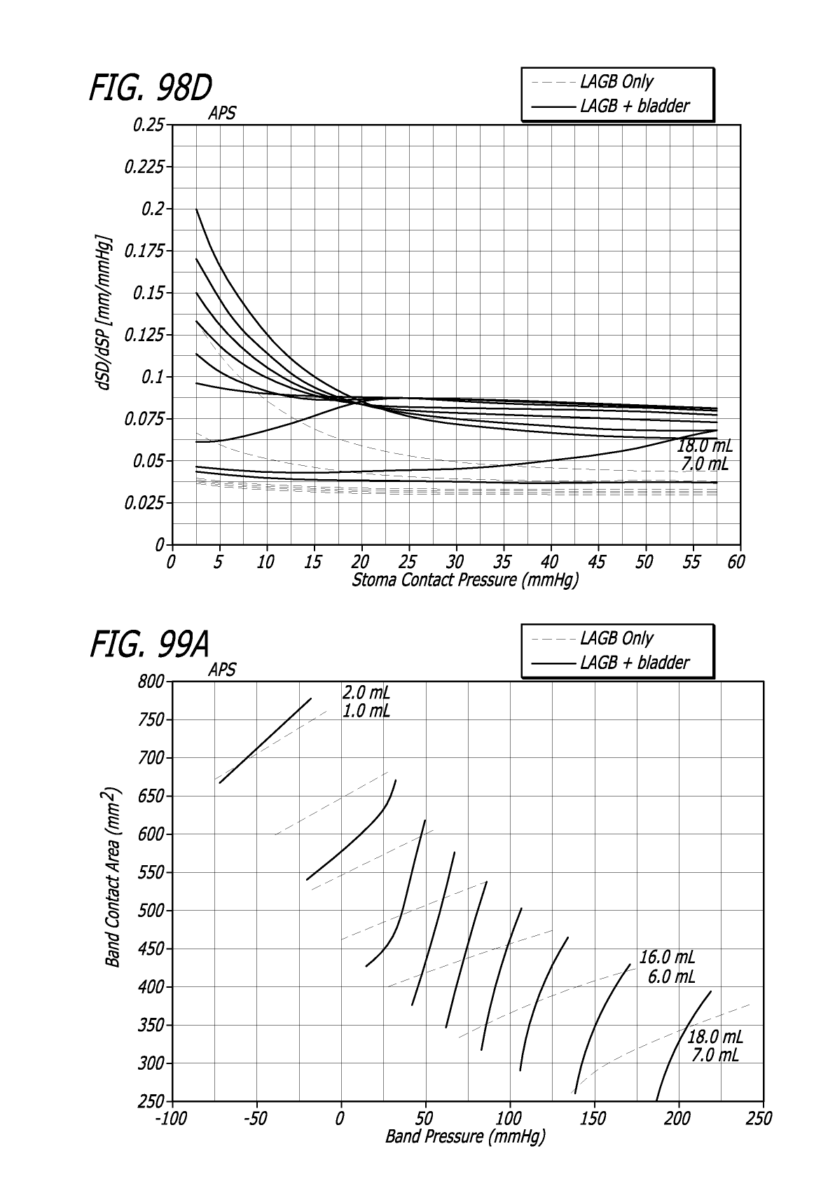

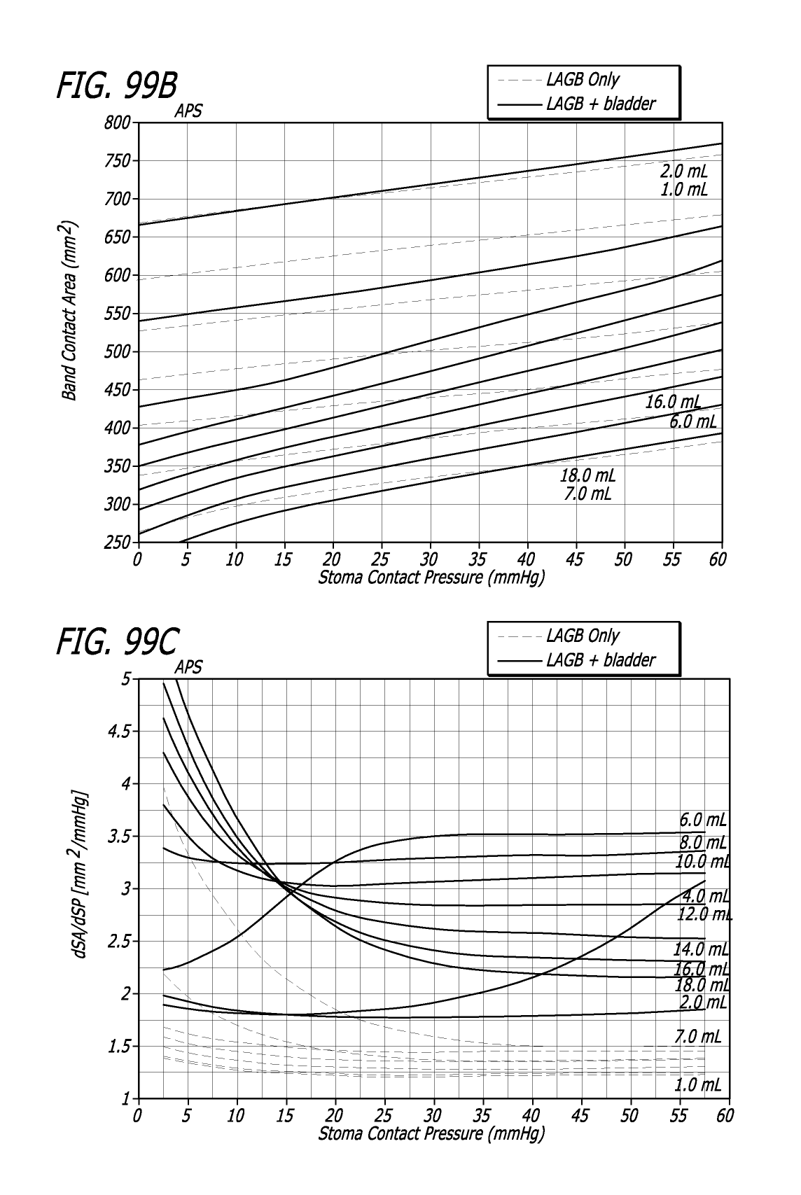

Further support for the increased distensibility of an LAGB plus bladder versus an LAGB only configuration, is found in FIGS. 96A-99C. The graphs in FIGS. 96A-99C represent in-silico models based on data derived from in-vitro bench experiments. The data in these graphs confirm the increased distensibility of an Ethicon SAGB-VC (Realize Band-C) coupled with a bladder and an Allergan APS (Lap-Band) coupled with a bladder versus the SAGB-VC and APS bands only.

While the invention has been illustrated and described herein in terms of its use as a bladder assembly connected to a gastric band, it will be apparent that the bladders disclosed herein can be used with any type of device that forms a restriction around a body part similar to a gastric band. Other modifications and improvements can be made without departing from the scope of the invention.

What is claimed:

1. A method of treating a patient having a gastric band assembly, comprising:
providing a gastric band assembly having a gastric band and a balloon portion, the balloon portion being in fluid communication with a bladder;
encircling stomach tissue with the balloon portion of the gastric band to form a band contact dimension; and
measuring band contact dimension distensibility (D) as a rate of band contact dimension change (ΔSD) per unit change in band contact pressure (ΔSP) according to the formula D=ΔSD/ΔSP.

2. The method of claim 1, wherein the band contact dimension is taken from a range of band contact diameters from 18 mm to 35 mm.

3. The method of claim 2, wherein the band contact pressure is taken from a range of band contact pressures from 15 mmHg to 100 mmHg.

4. The method of claim 2, wherein the measured distensibility (D) is greater than 0.05 mm/mmHg.

5. The method of claim 2, wherein the measured distensibility (D) is greater than 0.075 mm/mmHg.

6. The method of claim 1, wherein the band contact dimension is taken from a range of band contact areas from 250 mm² to 800 mm².

7. The method of claim 6, wherein the band contact pressure is taken from a range of band contact pressures from 15 mmHg to 100 mmHg.

8. The method of claim 6, wherein the measured distensibility (D) is greater than 1.6 mm²/mmHg.

9. The method of claim 6, wherein the measured distensibility (D) is greater than 2.6 mm²/mmHg.

10. A method of treating a patient having a gastric band assembly, comprising:
providing a gastric band assembly having a gastric band and a balloon portion, the balloon portion being in fluid communication with an assembly for increasing distensibility;
encircling stomach tissue with the balloon portion of the gastric band to form a band contact dimension;
enabling the band contact dimension to increase to a first distended size during a primary swallow peristalsis; and
enabling the band contact dimension to increase to a second distended size during any secondary swallow peristalses, wherein any subsequent distended size is greater than any previous distended size.

11. The method of claim 10, wherein the band contact dimension is a circumscribed area.

12. The method of claim 10, wherein the band contact dimension is diameter.

13. A method of treating a patient having a gastric band assembly, comprising:
providing a gastric band assembly having a gastric band and a balloon portion, the balloon portion being in fluid communication with an assembly for increasing distensibility;
encircling stomach tissue with the balloon portion of the gastric band filled with a basal fill volume;
enabling the fill volume within the balloon portion of the gastric band to decrease to a first post-swallow fill volume upon completion of a primary swallow peristalsis, wherein the first post-swallow fill volume is less than the basal fill volume.

14. The method of claim 13, wherein further enabling the fill volume within the balloon portion of the gastric band to decrease to subsequent post-swallow fill volumes upon completion of subsequent swallow peristalses, wherein any subsequent post-swallow fill volume is less than any previous post-swallow fill volume.

15. The method of claim 13, further comprising:
enabling the fill volume within the balloon portion of the gastric band to return to its basal fill volume after completion of all swallow peristalses.

16. A method of treating a patient having a gastric band assembly, comprising:
providing a gastric band assembly having a gastric band and a balloon portion, the balloon portion being in fluid communication with an assembly for increasing distensibility;
encircling stomach tissue with the balloon portion of the gastric band pressurized to a basal intra-band pressure magnitude;
enabling the intra-band pressure to increase to a first peak transient magnitude during a primary swallow peristalsis; and
enabling the intra-band pressure to increase to a subsequent peak transient magnitude during any subsequent swallow peristalses, wherein any subsequent peak transient magnitude is less than any previous peak transient magnitude.

17. A method of treating a patient having a gastric band assembly, comprising:
providing a gastric band assembly having a gastric band and a balloon portion, the balloon portion being in fluid communication with an assembly for increasing distensibility;
encircling stomach tissue with the balloon portion of the gastric band pressurized to a basal intra-band pressure magnitude; and
enabling the basal intra-band pressure to establish a first post-transient magnitude upon completion of a primary swallow peristalsis, wherein the first post-transient intra-band pressure magnitude is greater than the basal intra-band pressure magnitude.

18. The method of claim 17, wherein further enabling the intra-band pressure to establish subsequent post-transient magnitudes upon completion of subsequent swallow peristalses, wherein any subsequent post-transient intra-band pressure magnitude is greater than any previous post-transient intra-band pressure magnitude.

19. The method of claim 17, further comprising:
enabling the intra-band pressure within the gastric band to return to the basal intra-band pressure magnitude after completion of all swallow peristalses.

20. A method for determining band contact distensibility in a gastric band, comprising:
measuring a rate of band contact dimensional change (ΔSD) per unit change in applied band contact pressure (ΔSP) according to the formula
$$D = \frac{Δ SD}{Δ SP} ,$$
wherein band contact dimension (ΔSD) is band contact diameter; and
the band contact diameter distensibility (D) is greater than 0.075 mm/mmHg.

21. The method of claim 20, wherein the band contact diameter change is taken from a range of band contact diameters from 19 mm to 35 mm and the change in band contact pressure is taken from a pressure range from 5 mmHg to 150 mmHg.

22. The method of claim 20, wherein as the band contact pressure decreases, the band contact diameter decreases and the distensibility (D) remains greater than 0.075 mm/mmHg.

23. The method of claim 20, wherein as the band contact pressure increases, the band contact diameter increases and the distensibility (D) remains greater than 0.1 mm/mmHg.

24. A method for increasing band contact diameter distensibility in a gastric band, comprising:
providing a gastric band in fluid communication with a bladder;
increasing a band contact pressure by as much as 45 mmHg; and
increasing a band contact diameter by as much as 6.0 mm, thereby increasing distensibility of the gastric band with a bladder compared to a gastric band without a bladder.

25. A method for increasing distensibility of a stoma formed by a medical device, comprising:
providing a medical device configured to form a tissue stoma having a diameter in a mammalian body;
providing a bladder in fluid communication with the medical device;
measuring the rate of stoma diameter change (ΔSD) per unit change in an applied stoma contact pressure (ΔSP) according to the formula
$$D = \frac{Δ SD}{Δ SP} ,$$
where D is the distensibility of the stoma.

26. A medical device for treating a patient, comprising:
a gastric band assembly having a distensibility threshold greater than 0.075 mm/mmHg.

27. The medical device of claim 26, wherein the gastric band assembly comprises a gastric band and balloon, the balloon being in fluid communication with a bladder.

28. A medical device for treating a patient, comprising:
a gastric band assembly having a distensibility threshold greater than 1.6 mm²/mmHg.

29. The medical device of claim 28, wherein the gastric band assembly comprises a gastric band and balloon, the balloon being in fluid communication with a bladder.

30. A gastric band assembly, comprising:
a gastric band having a balloon, the balloon being in fluid communication with a bladder; and
the balloon and bladder having a distensibility greater than a distensibility of the gastric band and balloon without the bladder.

31. A medical device assembly, comprising:
a medical device assembly for restricting body tissue and having a first distensibility;
a bladder connected to the medical device assembly to form a system for restricting body tissue and having a second distensibility; and
the second distensibility being greater than the first distensibility.

32. A gastric band assembly, comprising:
a gastric band having a balloon for encircling stomach tissue to form a stoma, the balloon containing a baseline fluid level; and
increasing a band distensibility by enabling fluid to transiently exit the balloon.

33. The gastric band assembly of claim 32, wherein fluid returns to the balloon to the baseline fluid level.

34. A gastric band assembly, comprising:
a gastric band having a balloon and having a first band distensibility; and
a bladder in fluid communication with the balloon so that as fluid transfers from the balloon to the bladder, a second, increased effective band distensibility arises.

35. A method of treating a patient having a gastric band assembly, comprising:
providing a gastric band assembly having a gastric band and a balloon portion, the balloon portion being in fluid communication with an assembly for increasing distensibility;
encircling stomach tissue with the balloon portion of the gastric band to form a stoma dimension; and
enabling the stoma dimension to increase to a first distended size.

36. The method of claim 35, wherein the stoma dimension is area.

37. The method of claim 35, wherein the stoma dimension is diameter.
